(19) United States
(12) Patent Application Publication
(10) Pub. No.: US 2024/0216503 A1
(43) Pub. Date: Jul. 4, 2024

(54) METHODS OF TREATING ALLERGIES

(71) Applicant: Alladapt Immunotherapeutics, Inc., Menlo Park, CA (US)

(72) Inventors: Ashley Dombkowski, Woodside, CA (US); Dana McClintock, San Mateo, CA (US); Preeti Lal, Burlingame, CA (US); Amy Sullivan, El Granada, CA (US); Varghese Abraham, San Francisco, CA (US); Nusrat Rabbee, Washington, DC (US); James Kirchner, San Carlos, CA (US); Amelia Milani, San Francisco, CA (US)

(21) Appl. No.: 18/546,717

(22) PCT Filed: Feb. 23, 2022
(86) PCT No.: PCT/US2022/017566

§ 371 Date: Aug. 16, 2023

Related U.S. Application Data
(60) Provisional application No. 63/272,860, filed on Oct. 28, 2021.
(60) Provisional application No. 63/152,956, filed on Feb. 24, 2021.

(51) Int. Cl. A61K 39/35; A61K 39/00

(52) U.S. Cl. CPC A61K 39/35; A61K 2039/542; A61K 2039/545

(57) ABSTRACT

Provided are methods of treating or preventing allergies comprising administering mixed allergen compositions to subjects.

### CROSS REFERENCE

This application claims the benefit of and priority to U.S. Provisional Patent Application No. 63/152,956, filed on Feb. 24, 2021 and U.S. Provisional Patent Application No. 63/272,860, filed on Oct. 28, 2021, the entire disclosure of each of which is hereby incorporated by reference in its entirety for all purposes.

### BACKGROUND

Allergy is a disorder of the immune system characterized by the occurrence of allergic reactions to normally harmless environmental substances. Allergies are caused by allergens, which may be present in a wide variety of sources, including but not limited to pollens or other plant components, dust, molds or fungi, foods, additives, latex, transfusion reactions, animal or bird danders, insect venoms, radiocontrast medium, medications or chemicals. Common allergic reactions include eczema, hives, hay fever, asthma, and reactions to venoms. Mild allergies like hay fever are highly prevalent in the human population and cause symptoms such as allergic conjunctivitis, itchiness, and runny nose. In some people, severe allergies to environmental or dietary allergens or to medication may result in life-threatening anaphylactic reactions and potentially death, if left untreated.

A food allergy is an adverse immune response to a food allergen, e.g., a food protein. Common food allergens are found in shellfish, peanuts, tree nuts, fish, milk, eggs, soy and fresh fruits such as strawberries, mangoes, bananas, and apples. Immunoglobulin E (IgE)-mediated food allergies are classified as type-I immediate hypersensitivity reactions. These allergic reactions have an acute onset (from seconds to one hour) and the accompanying symptoms may include angioedema (soft tissue swelling of the eyelids, face, lips, tongue, larynx and trachea), hives, itching of the mouth, throat, eyes, or skin, gastrointestinal symptoms such as nausea, vomiting, diarrhea, stomach cramps, or abdominal pain, rhinorrhea or nasal congestion, wheezing, shortness of breath, or difficulty swallowing, and even anaphylaxis, a severe, whole-body allergic reaction that can result in death. It is estimated that 1 out of 12 children under 21 years of age have a doctor's diagnosis of food allergies, and over $24 billion is spent per year on health care costs for food allergic reactions, largely due to about 90,000 visits to the ER per year in the U.S. due to food induced anaphylactic symptoms. Moreover, there are still deaths that occur every year due to fatal food allergies.

Accordingly, there exists a need in the art for allergen compositions that can prevent and/or treat allergies, and methods for administering allergen compositions to prevent and/or treat allergies

### SUMMARY

The disclosure relates generally to methods and dosing regimens for administration of allergen compositions (e.g., mixed allergen compositions) to human subjects.

For example, in one aspect, provided herein is a method for (i) treating or preventing an allergy, and/or (ii) increasing the tolerance threshold of a food product in a subject having an allergy to the food product. The method comprises administering to the subject a mixed allergen composition.

In certain embodiments, a mixed allergen composition is administered to a subject orally. In certain embodiments, a mixed allergen composition is administered to a subject daily. In certain embodiments, a mixed allergen composition is administered to a subject for at least about 40 weeks.

In certain embodiments, a subject has a food allergy to only one food product. In certain embodiments, a subject has a food allergy to from two to fifteen (e.g., two to ten or two to five) different food products.

In certain embodiments, a dose of a mixed allergen composition is increased during the course of administration. For example, the dose of the mixed allergen composition may be increased about every two to six weeks.

In certain embodiments, a dose of a mixed allergen composition is increased from an initial dose (e.g., daily dose) of about 5 mg (by total protein weight) of the mixed allergen composition to a final dose (e.g., daily dose) that is the lower of about 4500 mg (by total protein weight) of the mixed allergen composition or a highest tolerated dose of the mixed allergen composition. For example, the dose of the mixed allergen composition may be increased from an initial dose (e.g., daily dose) of about 5 mg (by total protein weight) of the mixed allergen composition to about 20 mg, to about 50 mg, to about 100 mg, to about 200 mg, to about 400 mg, to about 700 mg, to about 1000 mg, to about 1500 mg, to about 3000 mg, to a final daily dose of about 4500 mg.

In certain embodiments, a dose of a mixed allergen composition is increased from an initial dose (e.g., daily dose) of about 5 mg (by total protein weight) of the mixed allergen composition to a final dose (e.g., daily dose) that is the lower of about 1500 mg (by total protein weight) of the mixed allergen composition or a highest tolerated dose of the mixed allergen composition. For example, the dose of the mixed allergen composition may be increased from an initial dose (e.g., daily dose) of about 5 mg (by total protein weight) of the mixed allergen composition, to about 20 mg, to about 50 mg, to about 100 mg, to about 200 mg, to about 400, mg to about 700 mg, to about 1000 mg, to a final daily dose of about 1500 mg.

In certain embodiments, a dose of a mixed allergen composition is increased from an initial dose (e.g., daily dose) of about 0.3 mg protein from each food source in the mixed allergen composition to a final dose (e.g., daily dose) that is the lower of about 300 mg of protein from each food source in the mixed allergen composition or a highest tolerated dose of the mixed allergen composition. For example, the dose of the mixed allergen composition may be increased from an initial dose (e.g., daily dose) of about 0.3 mg protein from each food source in the mixed allergen composition, to about 1.3 mg, to about 3.3 mg, to about 6.6 mg, to about 13.3 mg, to about 26.6 mg, to about 46.6 mg, to about 66.6 mg, to about 100 mg, to about 200 mg, to a final dose (e.g., daily dose) of about 300 mg protein from each food source in the mixed allergen composition.

In certain embodiments, a dose of a mixed allergen composition is increased from an initial dose (e.g., daily dose) of about 0.3 mg protein from each food source in the mixed allergen composition to a final dose (e.g., daily dose) that is the lower of about 100 mg of protein from each food source in the mixed allergen composition or a highest tolerated dose of the mixed allergen composition. For example, the dose of the mixed allergen composition may be increased from an initial dose (e.g., daily dose) of about 0.3 mg protein from each food source in the mixed allergen composition, to about 1.3 mg, to about 3.3 mg, to about 6.6 mg, to about 13.3 mg, to about 26.6 mg, to about 46.6 mg, to about 66.6 mg, to a final dose (e.g., daily dose) of about 100 mg protein from each food source in the mixed allergen composition.

In certain embodiments, a contemplated method comprises: (i) administering (e.g., orally) to a subject a dose (e.g., a daily dose) of about 5 mg (by total protein weight) of a mixed allergen composition for at least about two to six weeks; (ii) administering (e.g., orally) to the subject a dose (e.g., a daily dose) of about 20 mg of a mixed allergen composition for at least about two to six weeks; (iii) administering (e.g., orally) to the subject a dose (e.g., a daily dose) of about 50 mg of a mixed allergen composition for at least about two to six weeks; (iv) administering (e.g., orally) to the subject a dose (e.g., a daily dose) of about 100 mg of a mixed allergen composition for at least about two to six weeks; (v) administering (e.g., orally) to the subject a dose (e.g., a daily dose) of about 200 mg of a mixed allergen composition for at least about two to six weeks; (vi) administering (e.g., orally) to the subject a dose (e.g., a daily dose) of about 400 mg of a mixed allergen composition for at least about two to six weeks; (vii) administering (e.g., orally) to the subject a dose (e.g., a daily dose) of about 700 mg of a mixed allergen composition for at least about two to six weeks; (viii) administering (e.g., orally) to the subject a dose (e.g., a daily dose) of about 1000 mg of a mixed allergen composition for at least about two to six weeks; and/or (ix) administering (e.g., orally) to the subject a dose (e.g., a daily dose) of about 1500 mg of a mixed allergen composition for at least about two to six weeks. In certain embodiments, the method further comprises: (x) administering (e.g., orally) to the subject a dose (e.g., a daily dose) of about 3000 mg of a mixed allergen composition for at least about two to six weeks; and/or (xi) administering (e.g., orally) to the subject a dose (e.g., a daily dose) of about 4500 mg of a mixed allergen composition for at least about two to six weeks.

In certain embodiments, a contemplated method comprises: (i) administering (e.g., orally) to a subject a dose (e.g., a daily dose) of about 0.3 mg protein from each food source in a mixed allergen composition for at least about two to six weeks; (ii) administering (e.g., orally) to the subject a dose (e.g., a daily dose) of about 1.3 mg protein from each food source in the mixed allergen composition for at least about two to six weeks; (iii) administering (e.g., orally) to the subject a dose (e.g., a daily dose) of about 3.3 mg protein from each food source in the mixed allergen composition for at least about two to six weeks; (iv) administering (e.g., orally) to the subject a dose (e.g., a daily dose) of about 6.6 mg protein from each food source in the mixed allergen composition for at least about two to six weeks; (v) administering (e.g., orally) to the subject a dose (e.g., a daily dose) of about 13.3 mg protein from each food source in the mixed allergen composition for at least about two to six weeks; (vi) administering (e.g., orally) to the subject a dose (e.g., a daily dose) about 26.6 mg protein from each food source in the mixed allergen composition for at least about two to six weeks; (vii) administering (e.g., orally) to the subject a dose (e.g., a daily dose) of about 46.6 mg protein from each food source in the mixed allergen composition for at least about two to six weeks; (viii) administering (e.g., orally) to the subject a dose (e.g., a daily dose) of about 66.6 mg protein from each food source in the mixed allergen composition for at least about two to six weeks; and/or (ix) administering (e.g., orally) to the subject a dose (e.g., a daily dose) of about 100 mg protein from each food source in the mixed allergen composition for at least about two to six weeks. In certain embodiments, the method further comprises: (x) administering (e.g., orally) to the subject a dose (e.g., a daily dose) of about 200 mg protein from each food source in the mixed allergen composition for at least about two to six weeks; and/or (xi) administering (e.g., orally) to the subject a dose (e.g., a daily dose) of about 300 mg protein from each food source in the mixed allergen composition for at least about two to six weeks.

In certain embodiments of a contemplated method: (i) for about a first two to six weeks, a daily dose of a mixed allergen compositions is about 5 mg (by total protein weight); (ii) for about the next two to six weeks, the daily dose of the mixed allergen compositions is about 20 mg; (iii) for about the next two to six weeks, the daily dose of the mixed allergen compositions is about 50 mg; (iv) for about the next two to six weeks, the daily dose of the mixed allergen compositions is about 100 mg; (v) for about the next two to six weeks, the daily dose of the mixed allergen compositions is about 200 mg; (vi) for about the next two to six weeks, the daily dose of the mixed allergen compositions is about 400 mg; (vii) for about the next two to six weeks, the daily dose of the mixed allergen compositions is about 700 mg; (viii) for about the next two to six weeks, the daily dose of the mixed allergen compositions is about 1000 mg; (ix) for about the next two to six weeks, the daily dose of the mixed allergen compositions is about 1500 mg; (x) for about the next two to six weeks, the daily dose of the mixed allergen compositions is about 3000 mg; and (xi) for at least about the next twenty weeks, the daily dose of the mixed allergen compositions is about 4500 mg; wherein if a subject cannot tolerate a higher dose the dose is not increased and the subject is administered a highest tolerated dose for the remainder of the at least about 40 weeks.

In certain embodiments of a contemplated method: (i) for about a first two to six weeks, a daily dose of a mixed allergen compositions is about 5 mg (by total protein weight); (ii) for about the next two to six weeks, the daily dose of the mixed allergen compositions is about 20 mg; (iii) for about the next two to six weeks, the daily dose of the mixed allergen compositions is about 50 mg; (iv) for about the next two to six weeks, the daily dose of the mixed allergen compositions is about 100 mg; (v) for about the next two to six weeks, the daily dose of the mixed allergen compositions is about 200 mg; (vi) for about the next two to six weeks, the daily dose of the mixed allergen compositions is about 400 mg; (vii) for about the next two to six weeks, the daily dose of the mixed allergen compositions is about 700 mg; (viii) for about the next two to six weeks, the daily dose of the mixed allergen compositions is about 1000 mg; and (ix) for at least about the next 24 weeks, the daily dose of the mixed allergen compositions is about 1500 mg; wherein if a subject cannot tolerate a higher dose the dose is not increased and the subject is administered a highest tolerated dose for the remainder of the at least about 40 weeks.

In certain embodiments of a contemplated method: (i) for about a first two to six weeks, a daily dose of a mixed allergen compositions is about 0.3 mg protein from each food source in the mixed allergen composition; (ii) for about the next two to six weeks, the daily dose of the mixed allergen compositions is about 1.3 mg protein from each food source in the mixed allergen composition; (iii) for about the next two to six weeks, the daily dose of the mixed allergen compositions is about 3.3 mg protein from each food source in the mixed allergen composition; (iv) for about the next two to six weeks, the daily dose of the mixed allergen compositions is about 6.6 mg protein from each food source in the mixed allergen composition; (v) for about the next two to six weeks, the daily dose of the mixed allergen compositions is about 13.3 mg protein from each food source in the mixed allergen composition; (vi) for about the next two to six weeks, the daily dose of the mixed allergen compositions is about 26.6 mg protein from each food source in the mixed allergen composition; (vii) for about the next two to six weeks, the daily dose of the mixed allergen compositions is about 46.6 mg protein from each food source in the mixed allergen composition; (viii) for about the next two to six weeks, the daily dose of the mixed allergen compositions is about 66.6 mg protein from each food source in the mixed allergen composition; (ix) for about the next two to six weeks, the daily dose of the mixed allergen compositions is about 100 mg protein from each food source in the mixed allergen composition; (x) for about the next two to six weeks, the daily dose of the mixed allergen compositions is about 200 mg protein from each food source in the mixed allergen composition; and (xi) for about the next twenty weeks, the daily dose of the mixed allergen compositions is about 300 mg protein from each food source in the mixed allergen composition; wherein if a subject cannot tolerate a higher dose the dose is not increased and the subject is administered a highest tolerated dose for the remainder of the at least about 40 weeks.

In certain embodiments of a contemplated method: (i) for about a first two to six weeks, a daily dose of a mixed allergen compositions is about 0.3 mg protein from each food source in the mixed allergen composition; (ii) for about the next two to six weeks, the daily dose of the mixed allergen compositions is about 1.3 mg protein from each food source in the mixed allergen composition; (iii) for about the next two to six weeks, the daily dose of the mixed allergen compositions is about 3.3 mg protein from each food source in the mixed allergen composition; (iv) for about the next two to six weeks, the daily dose of the mixed allergen compositions is about 6.6 mg protein from each food source in the mixed allergen composition; (v) for about the next two to six weeks, the daily dose of the mixed allergen compositions is about 13.3 mg protein from each food source in the mixed allergen composition; (vi) for about the next two to six weeks, the daily dose of the mixed allergen compositions is about 26.6 mg protein from each food source in the mixed allergen composition; (vii) for about the next two to six weeks, the daily dose of the mixed allergen compositions is about 46.6 mg protein from each food source in the mixed allergen composition; (viii) for about the next two to six weeks, the daily dose of the mixed allergen compositions is about 66.6 mg protein from each food source in the mixed allergen composition; (ix) for about the next 24 weeks, the daily dose of the mixed allergen compositions is about 100 mg protein from each food source in the mixed allergen composition; wherein if a subject cannot tolerate a higher dose the dose is not increased and the subject is administered a highest tolerated dose for the remainder of the at least about 40 weeks.

In certain embodiments of a contemplated method: (i) for about a first two to six weeks, a daily dose of a mixed allergen compositions is about 5 mg (by total protein weight); (ii) for about the next two to six weeks, the daily dose of the mixed allergen compositions is about 20 mg; (iii) for about the next two to six weeks, the daily dose of the mixed allergen compositions is about 50 mg; (iv) if a subject can tolerate a daily dose of about 100 mg, for about the next two to six weeks, the daily dose of the mixed allergen compositions is about 100 mg, or, if the subject cannot tolerate a daily dose of about 100 mg, for about the next 30 to 34 weeks, the daily dose of the mixed allergen composition is about 50 mg; (v) if the subject can tolerate a daily dose of about 200 mg, for about the next two to six weeks, the daily dose of the mixed allergen compositions is about 200 mg, or, if the subject cannot tolerate a daily dose of about 200 mg, for about the next 28 to 32 weeks, the daily dose of the mixed allergen composition is about 100 mg; (vi) if the subject can tolerate a daily dose of about 400 mg, for about the next two to six weeks, the daily dose of the mixed allergen compositions is about 400 mg, or, if the subject cannot tolerate a daily dose of about 400 mg, for about the next 26 to 30 weeks, the daily dose of the mixed allergen composition is about 200 mg; (vii) if the subject can tolerate a daily dose of about 700 mg, for about the next two to six weeks, the daily dose of the mixed allergen compositions is about 700 mg, or, if the subject cannot tolerate a daily dose of about 700 mg, for about the next 24 to 28 weeks, the daily dose of the mixed allergen composition is about 400 mg; (viii) if the subject can tolerate a daily dose of about 1000 mg, for about the next two to six weeks, the daily dose of the mixed allergen compositions is about 1000 mg, or, if the subject cannot tolerate a daily dose of about 1000 mg, for about the next 22 to 26 weeks, the daily dose of the mixed allergen composition is about 700 mg; (ix) if the subject can tolerate a daily dose of about 1500 mg, for about the next two to six weeks, the daily dose of the mixed allergen compositions is about 1500 mg, or, if the subject cannot tolerate a daily dose of about 1500 mg, for about the next 20 to 24 weeks, the daily dose of the mixed allergen composition is about 1000 mg; (x) if the subject can tolerate a daily dose of about 3000 mg, for about the next two to six weeks, the daily dose of the mixed allergen compositions is about 3000 mg, or, if the subject cannot tolerate a daily dose of about 3000 mg, for about the next 18 to 22 weeks, the daily dose of the mixed allergen composition is about 1500 mg; and (xi) if the subject can tolerate a daily dose of about 4500 mg, for about the next 16 to 20 weeks, the daily dose of the mixed allergen compositions is about 4500 mg, or, if the subject cannot tolerate a daily dose of about 4500 mg, for about the next 16 to 20 weeks, the daily dose of the mixed allergen composition is about 3000 mg.

In certain embodiments of a contemplated method: (i) for about a first two to six weeks, a daily dose of a mixed allergen compositions is about 5 mg (by total protein weight); (ii) for about the next two to six weeks, the daily dose of the mixed allergen compositions is about 20 mg; (iii) for about the next two to six weeks, the daily dose of the mixed allergen compositions is about 50 mg; (iv) if a subject can tolerate a daily dose of about 100 mg, for about the next two to six weeks, the daily dose of the mixed allergen compositions is about 100 mg, or, if the subject cannot tolerate a daily dose of about 100 mg, for about the next 16 to 32 weeks, the daily dose of the mixed allergen composition is about 50 mg; (v) if the subject can tolerate a daily dose of about 200 mg, for about the next two to six weeks, the daily dose of the mixed allergen compositions is about 200 mg, or, if the subject cannot tolerate a daily dose of about 200 mg, for about the next 10 to 30 weeks, the daily dose of the mixed allergen composition is about 100 mg; (vi) if the subject can tolerate a daily dose of about 400 mg, for about the next two to six weeks, the daily dose of the mixed allergen compositions is about 400 mg, or, if the subject cannot tolerate a daily dose of about 400 mg, for about the next 4 to 28 weeks, the daily dose of the mixed allergen composition is about 200 mg; (vii) if the subject can tolerate a daily dose of about 700 mg, for about the next two to six weeks, the daily dose of the mixed allergen compositions is about 700 mg, or, if the subject cannot tolerate a daily dose of about 700 mg, for about the next 1 to 26 weeks, the daily dose of the mixed allergen composition is about 400 mg; (viii) if the subject can tolerate a daily dose of about 1000 mg, for about the next two to six weeks, the daily dose of the mixed allergen compositions is about 1000 mg, or, if the subject cannot tolerate a daily dose of about 1000 mg, for about the next 1 to 24 weeks, the daily dose of the mixed allergen composition is about 700 mg; (ix) if the subject can tolerate a daily dose of about 1500 mg, for about the next two to six weeks, the daily dose of the mixed allergen compositions is about 1500 mg, or, if the subject cannot tolerate a daily dose of about 1500 mg, for about the next 1 to 22 weeks, the daily dose of the mixed allergen composition is about 1000 mg; (x) if the subject can tolerate a daily dose of about 3000 mg, for about the next two to six weeks, the daily dose of the mixed allergen compositions is about 3000 mg, or, if the subject cannot tolerate a daily dose of about 3000 mg, for about the next 1 to 20 weeks, the daily dose of the mixed allergen composition is about 1500 mg; and (xi) if the subject can tolerate a daily dose of about 4500 mg, for about the next 1 to 20 weeks, the daily dose of the mixed allergen compositions is about 4500 mg, or, if the subject cannot tolerate a daily dose of about 4500 mg, for about the next 1 to 18 weeks, the daily dose of the mixed allergen composition is about 3000 mg.

In certain embodiments of a contemplated method: (i) for about a first two to six weeks, a daily dose of a mixed allergen compositions is about 5 mg (by total protein weight); (ii) for about the next two to six weeks, the daily dose of the mixed allergen compositions is about 20 mg; (iii) for about the next two to six weeks, the daily dose of the mixed allergen compositions is about 50 mg; (iv) if a subject can tolerate a daily dose of about 100 mg, for about the next two to six weeks, the daily dose of the mixed allergen compositions is about 100 mg, or, if the subject cannot tolerate a daily dose of about 100 mg, for about the next 30 to 34 weeks, the daily dose of the mixed allergen composition is about 50 mg; (v) if the subject can tolerate a daily dose of about 200 mg, for about the next two to six weeks, the daily dose of the mixed allergen compositions is about 200 mg, or, if the subject cannot tolerate a daily dose of about 200 mg, for about the next 28 to 32 weeks, the daily dose of the mixed allergen composition is about 100 mg; (vi) if the subject can tolerate a daily dose of about 400 mg, for about the next two to six weeks, the daily dose of the mixed allergen compositions is about 400 mg, or, if the subject cannot tolerate a daily dose of about 400 mg, for about the next 26 to 30 weeks, the daily dose of the mixed allergen composition is about 200 mg; (vii) if the subject can tolerate a daily dose of about 700 mg, for about the next two to six weeks, the daily dose of the mixed allergen compositions is about 700 mg, or, if the subject cannot tolerate a daily dose of about 700 mg, for about the next 24 to 28 weeks, the daily dose of the mixed allergen composition is about 400 mg; (viii) if the subject can tolerate a daily dose of about 1000 mg, for about the next two to six weeks, the daily dose of the mixed allergen compositions is about 1000 mg, or, if the subject cannot tolerate a daily dose of about 1000 mg, for about the next 22 to 26 weeks, the daily dose of the mixed allergen composition is about 700 mg; and (ix) if the subject can tolerate a daily dose of about 1500 mg, for about the next 20 to 24 weeks, the daily dose of the mixed allergen compositions is about 1500 mg, or, if the subject cannot tolerate a daily dose of about 1500 mg, for about the next 20 to 24 weeks, the daily dose of the mixed allergen composition is about 1000 mg.

In certain embodiments of a contemplated method: (i) for about a first two to six weeks, a daily dose of a mixed allergen compositions is about 5 mg (by total protein weight); (ii) for about the next two to six weeks, the daily dose of the mixed allergen compositions is about 20 mg; (iii) for about the next two to six weeks, the daily dose of the mixed allergen compositions is about 50 mg; (iv) if a subject can tolerate a daily dose of about 100 mg, for about the next two to six weeks, the daily dose of the mixed allergen compositions is about 100 mg, or, if the subject cannot tolerate a daily dose of about 100 mg, for about the next 16 to 32 weeks, the daily dose of the mixed allergen composition is about 50 mg; (v) if the subject can tolerate a daily dose of about 200 mg, for about the next two to six weeks, the daily dose of the mixed allergen compositions is about 200 mg, or, if the subject cannot tolerate a daily dose of about 200 mg, for about the next 10 to 30 weeks, the daily dose of the mixed allergen composition is about 100 mg; (vi) if the subject can tolerate a daily dose of about 400 mg, for about the next two to six weeks, the daily dose of the mixed allergen compositions is about 400 mg, or, if the subject cannot tolerate a daily dose of about 400 mg, for about the next 4 to 28 weeks, the daily dose of the mixed allergen composition is about 200 mg; (vii) if the subject can tolerate a daily dose of about 700 mg, for about the next two to six weeks, the daily dose of the mixed allergen compositions is about 700 mg, or, if the subject cannot tolerate a daily dose of about 700 mg, for about the next 1 to 26 weeks, the daily dose of the mixed allergen composition is about 400 mg; (viii) if the subject can tolerate a daily dose of about 1000 mg, for about the next two to six weeks, the daily dose of the mixed allergen compositions is about 1000 mg, or, if the subject cannot tolerate a daily dose of about 1000 mg, for about the next 1 to 24 weeks, the daily dose of the mixed allergen composition is about 700 mg; and (ix) if the subject can tolerate a daily dose of about 1500 mg, for about the next 1 to 24 weeks, the daily dose of the mixed allergen compositions is about 1500 mg, or, if the subject cannot tolerate a daily dose of about 1500 mg, for about the next 1 to 22 weeks, the daily dose of the mixed allergen composition is about 1000 mg.

In certain embodiments of a contemplated method: (i) for about a first two to six weeks, a daily dose of a mixed allergen compositions is about 0.3 mg protein from each food source in the mixed allergen composition; (ii) for about the next two to six weeks, the daily dose of the mixed allergen compositions is about 1.3 mg protein from each food source in the mixed allergen composition; (iii) for about the next two to six weeks, the daily dose of the mixed allergen compositions is about 3.3 mg protein from each food source in the mixed allergen composition; (iv) if a subject can tolerate a daily dose of about 6.6 mg protein from each food source in the mixed allergen composition, for about the next two to six weeks, the daily dose of the mixed allergen compositions is about 6.6 mg protein from each food source in the mixed allergen composition, or, if the subject cannot tolerate a daily dose of about 6.6 mg protein from each food source in the mixed allergen composition, for about the next 30 to 34 weeks, the daily dose of the mixed allergen composition is about 3.3 mg protein from each food source in the mixed allergen composition; (v) if the subject can tolerate a daily dose of about 13.3 mg protein from each food source in the mixed allergen composition, for about the next two to six weeks, the daily dose of the mixed allergen compositions is about 13.3 mg protein from each food source in the mixed allergen composition, or, if the subject cannot tolerate a daily dose of about 13.3 mg protein from each food source in the mixed allergen composition, for about the next 28 to 32 weeks, the daily dose of the mixed allergen composition is about 6.6 mg protein from each food source in the mixed allergen composition; (vi) if the subject can tolerate a daily dose of about 26.6 mg protein from each food source in the mixed allergen composition, for about the next two to six weeks, the daily dose of the mixed allergen compositions is about 26.6 mg protein from each food source in the mixed allergen composition, or, if the subject cannot tolerate a daily dose of about 26.6 mg protein from each food source in the mixed allergen composition, for about the next 26 to 30 weeks, the daily dose of the mixed allergen composition is about 13.3 mg protein from each food source in the mixed allergen composition; (vii) if the subject can tolerate a daily dose of about 46.6 mg protein from each food source in the mixed allergen composition, for about the next two to six weeks, the daily dose of the mixed allergen compositions is about 46.6 mg protein from each food source in the mixed allergen composition, or, if the subject cannot tolerate a daily dose of about 46.6 mg protein from each food source in the mixed allergen composition, for about the next 24 to 28 weeks, the daily dose of the mixed allergen composition is about 26.6 mg protein from each food source in the mixed allergen composition; (viii) if the subject can tolerate a daily dose of about 66.6 mg protein from each food source in the mixed allergen composition, for about the next two to six weeks, the daily dose of the mixed allergen compositions is about 66.6 mg protein from each food source in the mixed allergen composition, or, if the subject cannot tolerate a daily dose of about 66.6 mg protein from each food source in the mixed allergen composition, for about the next 22 to 26 weeks, the daily dose of the mixed allergen composition is about 46.6 mg protein from each food source in the mixed allergen composition; (ix) if the subject can tolerate a daily dose of about 100 mg protein from each food source in the mixed allergen composition, for about the next two to six weeks, the daily dose of the mixed allergen compositions is about 100 mg protein from each food source in the mixed allergen composition, or, if the subject cannot tolerate a daily dose of about 100 mg protein from each food source in the mixed allergen composition, for about the next 20 to 24 weeks, the daily dose of the mixed allergen composition is about 66.6 mg protein from each food source in the mixed allergen composition; (x) if the subject can tolerate a daily dose of about 200 mg protein from each food source in the mixed allergen composition, for about the next two to six weeks, the daily dose of the mixed allergen compositions is about 200 mg protein from each food source in the mixed allergen composition, or, if the subject cannot tolerate a daily dose of about 200 mg protein from each food source in the mixed allergen composition, for about the next 18 to 22 weeks, the daily dose of the mixed allergen composition is about 100 mg protein from each food source in the mixed allergen composition; and (xi) if the subject can tolerate a daily dose of about 300 mg protein from each food source in the mixed allergen composition, for about the next 16 to 20 weeks, the daily dose of the mixed allergen compositions is about 300 mg protein from each food source in the mixed allergen composition, or, if the subject cannot tolerate a daily dose of about 300 mg protein from each food source in the mixed allergen composition, for about the next 16 to 20 weeks, the daily dose of the mixed allergen composition is about 200 mg protein from each food source in the mixed allergen composition.

In certain embodiments of a contemplated method: (i) for about a first two to six weeks, a daily dose of a mixed allergen compositions is about 0.3 mg protein from each food source in the mixed allergen composition; (ii) for about the next two to six weeks, the daily dose of the mixed allergen compositions is about 1.3 mg protein from each food source in the mixed allergen composition; (iii) for about the next two to six weeks, the daily dose of the mixed allergen compositions is about 3.3 mg protein from each food source in the mixed allergen composition; (iv) if a subject can tolerate a daily dose of about 6.6 mg protein from each food source in the mixed allergen composition, for about the next two to six weeks, the daily dose of the mixed allergen compositions is about 6.6 mg protein from each food source in the mixed allergen composition, or, if the subject cannot tolerate a daily dose of about 6.6 mg protein from each food source in the mixed allergen composition, for about the next 16 to 32 weeks, the daily dose of the mixed allergen composition is about 3.3 mg protein from each food source in the mixed allergen composition; (v) if the subject can tolerate a daily dose of about 13.3 mg protein from each food source in the mixed allergen composition, for about the next two to six weeks, the daily dose of the mixed allergen compositions is about 13.3 mg protein from each food source in the mixed allergen composition, or, if the subject cannot tolerate a daily dose of about 13.3 mg protein from each food source in the mixed allergen composition, for about the next 10 to 30 weeks, the daily dose of the mixed allergen composition is about 6.6 mg protein from each food source in the mixed allergen composition; (vi) if the subject can tolerate a daily dose of about 26.6 mg protein from each food source in the mixed allergen composition, for about the next two to six weeks, the daily dose of the mixed allergen compositions is about 26.6 mg protein from each food source in the mixed allergen composition, or, if the subject cannot tolerate a daily dose of about 26.6 mg protein from each food source in the mixed allergen composition, for about the next 4 to 28 weeks, the daily dose of the mixed allergen composition is about 13.3 mg protein from each food source in the mixed allergen composition; (vii) if the subject can tolerate a daily dose of about 46.6 mg protein from each food source in the mixed allergen composition, for about the next two to six weeks, the daily dose of the mixed allergen compositions is about 46.6 mg protein from each food source in the mixed allergen composition, or, if the subject cannot tolerate a daily dose of about 46.6 mg protein from each food source in the mixed allergen composition, for about the next 1 to 26 weeks, the daily dose of the mixed allergen composition is about 26.6 mg protein from each food source in the mixed allergen composition; (viii) if the subject can tolerate a daily dose of about 66.6 mg protein from each food source in the mixed allergen composition, for about the next two to six weeks, the daily dose of the mixed allergen compositions is about 66.6 mg protein from each food source in the mixed allergen composition, or, if the subject cannot tolerate a daily dose of about 66.6 mg protein from each food source in the mixed allergen composition, for about the next 1 to 24 weeks, the daily dose of the mixed allergen composition is about 46.6 mg protein from each food source in the mixed allergen composition; (ix) if the subject can tolerate a daily dose of about 100 mg protein from each food source in the mixed allergen composition, for about the next two to six weeks, the daily dose of the mixed allergen compositions is about 100 mg protein from each food source in the mixed allergen composition, or, if the subject cannot tolerate a daily dose of about 100 mg protein from each food source in the mixed allergen composition, for about the next 1 to 22 weeks, the daily dose of the mixed allergen composition is about 66.6 mg protein from each food source in the mixed allergen composition; (x) if the subject can tolerate a daily dose of about 200 mg protein from each food source in the mixed allergen composition, for about the next two to six weeks, the daily dose of the mixed allergen compositions is about 200 mg protein from each food source in the mixed allergen composition, or, if the subject cannot tolerate a daily dose of about 200 mg protein from each food source in the mixed allergen composition, for about the next 1 to 20 weeks, the daily dose of the mixed allergen composition is about 100 mg protein from each food source in the mixed allergen composition; and (xi) if the subject can tolerate a daily dose of about 300 mg protein from each food source in the mixed allergen composition, for about the next 1 to 20 weeks, the daily dose of the mixed allergen compositions is about 300 mg protein from each food source in the mixed allergen composition, or, if the subject cannot tolerate a daily dose of about 300 mg protein from each food source in the mixed allergen composition, for about the next 1 to 18 weeks, the daily dose of the mixed allergen composition is about 200 mg protein from each food source in the mixed allergen composition.

In certain embodiments of a contemplated method: (i) for about a first two to six weeks, a daily dose of a mixed allergen compositions is about 0.3 mg protein from each food source in the mixed allergen composition; (ii) for about the next two to six weeks, the daily dose of the mixed allergen compositions is about 1.3 mg protein from each food source in the mixed allergen composition; (iii) for about the next two to six weeks, the daily dose of the mixed allergen compositions is about 3.3 mg protein from each food source in the mixed allergen composition; (iv) if a subject can tolerate a daily dose of about 6.6 mg protein from each food source in the mixed allergen composition, for about the next two to six weeks, the daily dose of the mixed allergen compositions is about 6.6 mg protein from each food source in the mixed allergen composition, or, if the subject cannot tolerate a daily dose of about 6.6 mg protein from each food source in the mixed allergen composition, for about the next 30 to 34 weeks, the daily dose of the mixed allergen composition is about 3.3 mg protein from each food source in the mixed allergen composition; (v) if the subject can tolerate a daily dose of about 13.3 mg protein from each food source in the mixed allergen composition, for about the next two to six weeks, the daily dose of the mixed allergen compositions is about 13.3 mg protein from each food source in the mixed allergen composition, or, if the subject cannot tolerate a daily dose of about 13.3 mg protein from each food source in the mixed allergen composition, for about the next 28 to 32 weeks, the daily dose of the mixed allergen composition is about 6.6 mg protein from each food source in the mixed allergen composition; (vi) if the subject can tolerate a daily dose of about 26.6 mg protein from each food source in the mixed allergen composition, for about the next two to six weeks, the daily dose of the mixed allergen compositions is about 26.6 mg protein from each food source in the mixed allergen composition, or, if the subject cannot tolerate a daily dose of about 26.6 mg protein from each food source in the mixed allergen composition, for about the next 26 to 30 weeks, the daily dose of the mixed allergen composition is about 13.3 mg protein from each food source in the mixed allergen composition; (vii) if the subject can tolerate a daily dose of about 46.6 mg protein from each food source in the mixed allergen composition, for about the next two to six weeks, the daily dose of the mixed allergen compositions is about 46.6 mg protein from each food source in the mixed allergen composition, or, if the subject cannot tolerate a daily dose of about 46.6 mg protein from each food source in the mixed allergen composition, for about the next 24 to 28 weeks, the daily dose of the mixed allergen composition is about 26.6 mg protein from each food source in the mixed allergen composition; (viii) if the subject can tolerate a daily dose of about 66.6 mg protein from each food source in the mixed allergen composition, for about the next two to six weeks, the daily dose of the mixed allergen compositions is about 66.6 mg protein from each food source in the mixed allergen composition, or, if the subject cannot tolerate a daily dose of about 66.6 mg protein from each food source in the mixed allergen composition, for about the next 22 to 26 weeks, the daily dose of the mixed allergen composition is about 46.6 mg protein from each food source in the mixed allergen composition; and (ix) if the subject can tolerate a daily dose of about 100 mg protein from each food source in the mixed allergen composition, for about the next 20 to 24 weeks, the daily dose of the mixed allergen compositions is about 100 mg protein from each food source in the mixed allergen composition, or, if the subject cannot tolerate a daily dose of about 100 mg protein from each food source in the mixed allergen composition, for about the next 20 to 24 weeks, the daily dose of the mixed allergen composition is about 66.6 mg protein from each food source in the mixed allergen composition.

In certain embodiments of a contemplated method: (i) for about a first two to six weeks, a daily dose of a mixed allergen compositions is about 0.3 mg protein from each food source in the mixed allergen composition; (ii) for about the next two to six weeks, the daily dose of the mixed allergen compositions is about 1.3 mg protein from each food source in the mixed allergen composition; (iii) for about the next two to six weeks, the daily dose of the mixed allergen compositions is about 3.3 mg protein from each food source in the mixed allergen composition; (iv) if a subject can tolerate a daily dose of about 6.6 mg protein from each food source in the mixed allergen composition, for about the next two to six weeks, the daily dose of the mixed allergen compositions is about 6.6 mg protein from each food source in the mixed allergen composition, or, if the subject cannot tolerate a daily dose of about 6.6 mg protein from each food source in the mixed allergen composition, for about the next 16 to 32 weeks, the daily dose of the mixed allergen composition is about 3.3 mg protein from each food source in the mixed allergen composition; (v) if the subject can tolerate a daily dose of about 13.3 mg protein from each food source in the mixed allergen composition, for about the next two to six weeks, the daily dose of the mixed allergen compositions is about 13.3 mg protein from each food source in the mixed allergen composition, or, if the subject cannot tolerate a daily dose of about 13.3 mg protein from each food source in the mixed allergen composition, for about the next 10 to 30 weeks, the daily dose of the mixed allergen composition is about 6.6 mg protein from each food source in the mixed allergen composition; (vi) if the subject can tolerate a daily dose of about 26.6 mg protein from each food source in the mixed allergen composition, for about the next two to six weeks, the daily dose of the mixed allergen compositions is about 26.6 mg protein from each food source in the mixed allergen composition, or, if the subject cannot tolerate a daily dose of about 26.6 mg protein from each food source in the mixed allergen composition, for about the next 4 to 28 weeks, the daily dose of the mixed allergen composition is about 13.3 mg protein from each food source in the mixed allergen composition; (vii) if the subject can tolerate a daily dose of about 46.6 mg protein from each food source in the mixed allergen composition, for about the next two to six weeks, the daily dose of the mixed allergen compositions is about 46.6 mg protein from each food source in the mixed allergen composition, or, if the subject cannot tolerate a daily dose of about 46.6 mg protein from each food source in the mixed allergen composition, for about the next 1 to 26 weeks, the daily dose of the mixed allergen composition is about 26.6 mg protein from each food source in the mixed allergen composition; (viii) if the subject can tolerate a daily dose of about 66.6 mg protein from each food source in the mixed allergen composition, for about the next two to six weeks, the daily dose of the mixed allergen compositions is about 66.6 mg protein from each food source in the mixed allergen composition, or, if the subject cannot tolerate a daily dose of about 66.6 mg protein from each food source in the mixed allergen composition, for about the next 1 to 24 weeks, the daily dose of the mixed allergen composition is about 46.6 mg protein from each food source in the mixed allergen composition; and (ix) if the subject can tolerate a daily dose of about 100 mg protein from each food source in the mixed allergen composition, for about the next 1 to 24 weeks, the daily dose of the mixed allergen compositions is about 100 mg protein from each food source in the mixed allergen composition, or, if the subject cannot tolerate a daily dose of about 100 mg protein from each food source in the mixed allergen composition, for about the next 1 to 22 weeks, the daily dose of the mixed allergen composition is about 66.6 mg protein from each food source in the mixed allergen composition.

In certain embodiments of a contemplated method, a subject is administered (e.g., orally) a dose (e.g., a daily dose) of a mixed allergen composition for at least about a next 1, 2, 3, or 4 weeks, 1, 2, 3, 4, 5, 6, 9, or 12 months or 1, 2, 3, 4, 5 years, for example, a dose of (i) about 4500 mg of the mixed allergen composition, (ii) about 1500 mg of the mixed allergen composition, or (iii) the highest tolerated dose of the mixed allergen composition.

A contemplated mixed allergen composition may comprise, for example, an allergen from at least 6, 7, 8, 9, 10, 11, 12, 13, 14, or 15 different food sources. In certain embodiments, a mixed allergen composition comprises an almond, cashew, cod, egg, hazelnut, milk, peanut, pecan, pistachio, salmon, sesame, shrimp, soy, walnut, or wheat (e.g., wheat gluten) allergen, or any combination thereof. In certain embodiments, a mixed allergen composition comprises almond protein, cashew protein, cod powder, dried eggs, hazelnut flour, milk protein, peanut flour, pecan flour, pistachio flour, salmon powder, sesame flour, shrimp powder, soy protein, walnut flour, or wheat (e.g., wheat gluten), or any combination thereof.

In certain embodiments of a contemplated method, one or more of the doses of a mixed allergen composition comprises about equal parts per weight of protein from each food source present in the mixed allergen composition. In certain embodiments, one or more of the doses of the mixed allergen composition comprises: about 0.3 mg, about 1.3 mg, about 6.6 mg, about 13.3 mg, about 26.6 mg, about 46.6 mg, about 66.6 mg about 100 mg, about 200 mg, or about 300 mg of protein from each food source present in the mixed allergen composition; and/or about 5 mg, about 20 mg, about 50 mg, about 100 mg, about 200 mg, about 400 mg, about 700 mg, about 1000 mg, about 1500 mg, about 3000 mg, or about 4000 mg of total food protein.

In certain embodiments of a contemplated method, an amount of one or more (e.g., one, two, three, four, five, or more than five) allergic foods that the subject can tolerate is increased. The amount of an allergic food that the subject can tolerate may be measured, for example, by a double-blind, placebo-controlled food challenge (DBPCFC). As a DBPCFC includes feeding of multiple, gradually increasing doses of a suspected allergenic food to a subject, tolerance may be measured by the highest individual dose of food consumed during a DBPCFC with no or only mild symptoms, or the total, cumulative amount of food consumed during a DBPCFC with no or only mild symptoms. For example, a contemplated method may: (i) increase a highest amount of an allergenic food that a subject can tolerate in a DBPCFC from less than or equal to about 100 mg (by protein weight) to greater than or equal to about 300 mg; (ii) increase a highest amount of an allergenic food that a subject can tolerate in a DBPCFC from less than or equal to about 100 mg (by protein weight) to greater than or equal to about 600 mg; (iii) increase a highest amount of an allergenic food that a subject can tolerate in a DBPCFC from less than or equal to about 100 mg (by protein weight) to greater than or equal to about 1000 mg; (iv) increase a highest amount of an allergenic food that a subject can tolerate in a DBPCFC from less than or equal to about 100 mg (by protein weight) to greater than or equal to about 2000 mg; and/or (v) increase a highest amount of an allergenic food that a subject can tolerate in a DBPCFC from less than or equal to about 100 mg (by protein weight) to greater than or equal to about 4000 mg. A contemplated method may: (i) increase a total amount of an allergenic food that a subject can tolerate in a DBPCFC from less than or equal to about 100 or 144 mg (by protein weight) to greater than or equal to about 400 or 444 mg; (ii) increase a total amount of an allergenic food that a subject can tolerate in a DBPCFC from less than or equal to about 100 or 144 mg (by protein weight) to greater than or equal to about 1000 or 1044 mg; (iii) increase a total amount of an allergenic food that a subject can tolerate in a DBPCFC from less than or equal to about 100 or 144 mg (by protein weight) to greater than or equal to about 2000 or 2044 mg; (iv) increase a total amount of an allergenic food that a subject can tolerate in a DBPCFC from less than or equal to about 100 or 144 mg (by protein weight) to greater than or equal to about 4000 or 4044 mg; and/or (v) increase a total amount of an allergenic food that a subject can tolerate in a DBPCFC from less than or equal to about 100 or 144 mg (by protein weight) to greater than or equal to about 8000 or 8044 mg.

In certain embodiments of a contemplated method, a subject is from about 4 years to about 55 years old. In certain embodiments, a subject is a pediatric subject. In certain embodiments, a subject is allergenic to one or more foods selected from almond, cashew, chicken's egg, codfish, cow's milk, hazelnut, peanut, pecan, pistachio, salmon, sesame seed, shrimp, soy, walnut, and wheat (e.g., wheat gluten). For example, in certain embodiments, a subject cannot tolerate 100 mg (by protein weight), as measured by DBPCFC, of one or more foods selected from almond, cashew, chicken's egg, codfish, cow's milk, hazelnut, peanut, pecan, pistachio, salmon, sesame seed, shrimp, soy, walnut, and wheat (e.g., wheat gluten). In certain embodiments, a subject is allergic to at most five foods selected from almond, cashew, chicken's egg, codfish, cow's milk, hazelnut, peanut, pecan, pistachio, salmon, sesame seed, shrimp, soy, walnut, and wheat (e.g., wheat gluten).

In certain embodiments of a contemplated method, a subject (i) has not had a severe/life-threatening episode of anaphylaxis/anaphylactic shock within 60 days of receiving the initial dose of the mixed allergen composition; (ii) does not have a history of eosinophilic esophagitis (EoE), other eosinophilic gastrointestinal disease, chronic, recurrent, or severe gastrointestinal reflux disease (GERD), symptoms of dysphagia (e.g., difficulty swallowing, food “getting stuck”), or recurrent gastrointestinal symptoms of undiagnosed etiology; (iii) does not have a history of chronic disease (other than asthma, atopic dermatitis, or allergic rhinitis) that is, or is at significant risk of becoming unstable or requiring a change in chronic therapeutic regimen; and/or (iv) has not had an active infection within 30 days of receiving the initial dose of the mixed allergen composition.

In certain embodiments of a contemplated method, each dose of the mixed allergen composition is administered to the subject in a clinic. Alternatively, for example, when a subject has a mild to moderate food allergy, one or more doses of the mixed allergen composition may be administered to the subject elsewhere. For example, one or more doses of the mixed allergen composition may be administered at the subject's home, and/or via telemedicine. Similarly, up-dosing of the mixed allergen composition may be performed in a clinic or elsewhere. For example, a dose of the mixed allergen composition may be increased at the subject's home, and/or via telemedicine.

In another aspect, provided herein is a kit comprising: (i) at least about 14 five mg (by total protein weight) doses of a mixed allergen composition; (ii) at least about 14 20 mg doses of the mixed allergen composition; (iii) at least about 14 50 mg doses of the mixed allergen composition; (iv) at least about 14 100 mg doses of the mixed allergen composition; (v) at least about 14 200 mg doses of the mixed allergen composition; (vi) at least about 14 400 mg doses of the mixed allergen composition; (vii) at least about 14 700 mg doses of the mixed allergen composition; (viii) at least about 14 1000 mg doses of the mixed allergen composition; and (ix) at least about 14 1500 mg doses of the mixed allergen composition. In certain embodiments, a contemplated kit further comprises: (x) at least about 14 3000 mg doses of the mixed allergen composition; and (xi) at least about 14 4500 mg doses of the mixed allergen composition.

In another aspect, provided herein is a kit comprising: (i) at least about 21 five mg (by total protein weight) doses of a mixed allergen composition; (ii) at least about 21 20 mg doses of the mixed allergen composition; (iii) at least about 21 50 mg doses of the mixed allergen composition; (iv) at least about 21 100 mg doses of the mixed allergen composition; (v) at least about 21 200 mg doses of the mixed allergen composition; (vi) at least about 21 400 mg doses of the mixed allergen composition; (vii) at least about 21 700 mg doses of the mixed allergen composition; (viii) at least about 21 1000 mg doses of the mixed allergen composition; and (ix) at least about 21 1500 mg doses of the mixed allergen composition. In certain embodiments, a contemplated kit further comprises: (x) at least about 21 3000 mg doses of the mixed allergen composition; and (xi) at least about 21 4500 mg doses of the mixed allergen composition.

In another aspect, provided herein is a kit comprising: at least 70 containers each comprising about 5 mg (by total protein weight) of a mixed allergen composition; at least 42 containers each comprising about 50 mg (by total protein weight) of the mixed allergen composition; at least 56 containers each comprising about 200 mg (by total protein weight) of the mixed allergen composition; at least 42 containers each comprising about 500 mg (by total protein weight) of the mixed allergen composition; and at least 14 containers each comprising about 1500 mg (by total protein weight) of the mixed allergen composition. In certain embodiments, a contemplated kit further comprises at least an additional 70 containers each comprising about 1500 mg (by total protein weight) of the mixed allergen composition.

In another aspect, provided herein is a kit comprising: at least 105 containers each comprising about 5 mg (by total protein weight) of a mixed allergen composition; at least 63 containers each comprising about 50 mg (by total protein weight) of the mixed allergen composition; at least 84 containers each comprising about 200 mg (by total protein weight) of the mixed allergen composition; at least 63 containers each comprising about 500 mg (by total protein weight) of the mixed allergen composition; and at least 21 containers each comprising about 1500 mg (by total protein weight) of the mixed allergen composition. In certain embodiments, a contemplated kit further comprises at least an additional 105 containers each comprising about 1500 mg (by total protein weight) of the mixed allergen composition.

In another aspect, provided herein is a kit comprising at least 14 containers each comprising about 5 mg (by total protein weight) of a mixed allergen composition.

In another aspect, provided herein is a kit comprising at least 21 containers each comprising about 5 mg (by total protein weight) of a mixed allergen composition.

In another aspect, provided herein is a kit comprising at least 56 containers each comprising about 5 mg (by total protein weight) of a mixed allergen composition. In certain embodiments, a contemplated kit comprises 4 secondary containers, which each comprise at least 14 primary containers each comprising about 5 mg (by total protein weight) of the mixed allergen composition.

In another aspect, provided herein is a kit comprising at least 84 containers each comprising about 5 mg (by total protein weight) of a mixed allergen composition. In certain embodiments, a contemplated kit comprises 4 secondary containers, which each comprise at least 21 primary containers each comprising about 5 mg (by total protein weight) of the mixed allergen composition.

In another aspect, provided herein is a kit comprising at least 14 containers each comprising about 50 mg (by total protein weight) of a mixed allergen composition.

In another aspect, provided herein is a kit comprising at least 21 containers each comprising about 50 mg (by total protein weight) of a mixed allergen composition.

In another aspect, provided herein is a kit comprising at least 28 containers each comprising about 50 mg (by total protein weight) of a mixed allergen composition. In certain embodiments, a contemplated kit comprises 2 secondary containers, which each comprise at least 14 primary containers each comprising about 50 mg (by total protein weight) of the mixed allergen composition.

In another aspect, provided herein is a kit comprising at least 42 containers each comprising about 50 mg (by total protein weight) of a mixed allergen composition. In certain embodiments, a contemplated kit comprises 2 secondary containers, which each comprise at least 21 primary containers each comprising about 50 mg (by total protein weight) of the mixed allergen composition.

In another aspect, provided herein is a kit comprising at least 14 containers each comprising about 200 mg (by total protein weight) of a mixed allergen composition.

In another aspect, provided herein is a kit comprising at least 21 containers each comprising about 200 mg (by total protein weight) of a mixed allergen composition.

In another aspect, provided herein is a kit comprising at least 28 containers each comprising about 200 mg (by total protein weight) of a mixed allergen composition. In certain embodiments, a contemplated kit comprises 2 secondary containers, which each comprise at least 14 primary containers each comprising about 200 mg (by total protein weight) of the mixed allergen composition.

In another aspect, provided herein is a kit comprising at least 42 containers each comprising about 200 mg (by total protein weight) of a mixed allergen composition. In certain embodiments, a contemplated kit comprises 2 secondary containers, which each comprise at least 21 primary containers each comprising about 200 mg (by total protein weight) of the mixed allergen composition.

In another aspect, provided herein is a kit comprising at least 14 containers each comprising about 200 mg (by total protein weight) of a mixed allergen composition and at least 14 containers each comprising about 500 mg (by total protein weight) of the mixed allergen composition. In certain embodiments, a contemplated kit comprises a first secondary container which comprises at least 14 primary containers each comprising about 200 mg (by total protein weight) of the mixed allergen composition; and a second secondary container which comprises at least 14 primary containers each comprising about 500 mg (by total protein weight) of the mixed allergen composition.

In another aspect, provided herein is a kit comprising at least 21 containers each comprising about 200 mg (by total protein weight) of a mixed allergen composition and at least 21 containers each comprising about 500 mg (by total protein weight) of the mixed allergen composition. In certain embodiments, a contemplated kit comprises a first secondary container which comprises at least 21 primary containers each comprising about 200 mg (by total protein weight) of the mixed allergen composition; and a second secondary container which comprises at least 21 primary containers each comprising about 500 mg (by total protein weight) of the mixed allergen composition.

In another aspect, provided herein is a kit comprising at least 28 containers each comprising about 500 mg (by total protein weight) of a mixed allergen composition. In certain embodiments, a contemplated kit comprises 2 secondary containers, which each comprise at least 14 primary containers each comprising about 500 mg (by total protein weight) of the mixed allergen composition.

In another aspect, provided herein is a kit comprising at least 42 containers each comprising about 500 mg (by total protein weight) of a mixed allergen composition. In certain embodiments, a contemplated kit comprises 2 secondary containers, which each comprise at least 21 primary containers each comprising about 500 mg (by total protein weight) of the mixed allergen composition.

In another aspect, provided herein is a kit comprising at least 14 containers each comprising about 1500 mg (by total protein weight) of a mixed allergen composition.

In another aspect, provided herein is a kit comprising at least 21 containers each comprising about 1500 mg (by total protein weight) of a mixed allergen composition.

In another aspect, provided herein is a kit comprising at least 28 containers each comprising about 1500 mg (by total protein weight) of a mixed allergen composition. In certain embodiments, a contemplated kit comprises 2 secondary containers, which each comprise at least 14 primary containers each comprising about 1500 mg (by total protein weight) of the mixed allergen composition.

In another aspect, provided herein is a kit comprising at least 42 containers each comprising about 1500 mg (by total protein weight) of a mixed allergen composition. In certain embodiments, a contemplated kit comprises 2 secondary containers, which each comprise at least 21 primary containers each comprising about 1500 mg (by total protein weight) of the mixed allergen composition.

In another aspect, provided herein is a kit comprising at least 42 containers each comprising about 1500 mg (by total protein weight) of a mixed allergen composition. In certain embodiments, a contemplated kit comprises 3 secondary containers, which each comprise at least 14 primary containers each comprising about 1500 mg (by total protein weight) of the mixed allergen composition.

In another aspect, provided herein is a kit comprising at least 63 containers each comprising about 1500 mg (by total protein weight) of a mixed allergen composition. In certain embodiments, a contemplated kit comprises 2 secondary containers, which each comprise at least 21 primary containers each comprising about 1500 mg (by total protein weight) of the mixed allergen composition.

In another aspect, provided herein is a kit comprising at least 14 containers each comprising about 5 mg (by total protein weight) of a mixed allergen composition. In another aspect, provided herein is a kit comprising at least 14 containers each comprising about 50 mg (by total protein weight) of a mixed allergen composition. In another aspect, provided herein is a kit comprising at least 14 containers each comprising about 200 mg (by total protein weight) of a mixed allergen composition. In another aspect, provided herein is a kit comprising at least 14 containers each comprising about 500 mg (by total protein weight) of a mixed allergen composition. In another aspect, provided herein is a kit comprising at least 14 containers each comprising about 1500 mg (by total protein weight) of a mixed allergen composition. In another aspect, provided herein is a kit comprising at least 21 containers each comprising about 5 mg (by total protein weight) of a mixed allergen composition. In another aspect, provided herein is a kit comprising at least 21 containers each comprising about 50 mg (by total protein weight) of a mixed allergen composition. In another aspect, provided herein is a kit comprising at least 21 containers each comprising about 200 mg (by total protein weight) of a mixed allergen composition. In another aspect, provided herein is a kit comprising at least 21 containers each comprising about 500 mg (by total protein weight) of a mixed allergen composition. In another aspect, provided herein is a kit comprising at least 21 containers each comprising about 1500 mg (by total protein weight) of a mixed allergen composition.

### BRIEF DESCRIPTION OF THE DRAWINGS

FIG. 1 is schematic of the study design described in Example 1. Abbreviations: DBPCFC=double-blind, placebo-controlled food challenge; EOT=End of Treatment; OLE=open-label extension.

FIG. 2 is a depiction of a method to determine individual food DBPCFC testing for initial screening of subjects. Abbreviations: DBPCFC=double-blind, placebo-controlled food challenge; SPT=skin-prick test.

FIG. 3 is a depiction of a method for categorizing individual foods in formulation A for each subject based on study entry criteria and results of screening DBPCFC. Abbreviations: DBPCFC=double-blind, placebo-controlled food challenge.

FIG. 4 is a depiction of a method to determine individual food DBPCFC testing at an exit evaluation (e.g., week 40 visit) of subjects. Abbreviations: DBPCFC=double-blind, placebo-controlled food challenge; SPT=skin-prick test.

### DETAILED DESCRIPTION

The disclosure relates generally to methods and dosing regimens for administration of allergen compositions (e.g., mixed allergen compositions) to human subjects.

### I. Therapeutic Methods

Compositions and methods disclosed herein can be used to treat or prevent various allergic disorders in a subject. A contemplated method comprises administering to a subject an effective amount of an allergen composition (e.g., a mixed allergen composition) disclosed herein, to treat an allergic disorder in the subject. Exemplary allergic disorders include asthma, atopic dermatitis, allergic rhinitis, allergic conjunctivitis, food allergies (e.g., single or multiple food allergies), insect and/or bee allergies, animal allergies, environmental allergies, or drug allergies.

Compositions and methods disclosed herein can also be used to increase the tolerance threshold of a food product in a subject having an allergy to the food product. A contemplated method comprises administering to the subject an effective amount of an allergen composition (e.g., a mixed allergen composition) disclosed herein to increase the tolerance threshold of the food product.

As used herein, “treat”, “treating” and “treatment” mean the treatment of a disease in a subject, e.g., in a human. This includes: (a) inhibiting the disease, i.e., arresting its development; and (b) relieving the disease, i.e., causing regression of the disease state. Contemplated methods may reduce or eliminate allergic symptoms including, for example: eczema, asthma, atopic dermatitis, bronchospasm, cough, rhinorrhea, angioedema, gastric hypermotility, urticaria (hives), pruritis, fatigue, bradycardia, allergic rhinitis, allergic conjunctivitis, and/or hypotension.

As used herein, the terms “subject” and “patient” refer to an organism to be treated by the methods and compositions described herein. Such organisms preferably include, but are not limited to, mammals, e.g., human, a companion animal (e.g., dog, cat, or rabbit), or a livestock animal (for example, cow, sheep, pig, goat, horse, donkey, and mule, buffalo, oxen, or camel)). In certain embodiments, a subject is a pediatric subject (e.g., less than or equal to 18 years old).

It is understood that treatment or prevention of an allergy may include, for example, treatment or prevention of a primary allergy (an allergy to an allergen resulting from exposure to the allergen) or a secondary allergy (an allergy to an allergen resulting from exposure to a different, cross-reactive allergen).

In certain embodiments of a contemplated method, a subject has an allergy, for example, one or more food allergies. In certain embodiments, a subject is allergic to 1, 2, 3, 4, 5, 6, 7, 8, 9, 10, 11, 12, 13, 14, 15, or more than 15 food products. In certain embodiments, a subject is allergic to from 1 to 15, 1 to 14, 1 to 13, 1 to 12, 1 to 11, 1 to 10, 1 to 9, 1 to 8, 1 to 7, 1 to 6, 1 to 5, 1 to 4, 1 to 3, 1 to 2, 2 to 15, 2 to 14, 2 to 13, 2 to 12, 2 to 11, 2 to 10, 2 to 9, 2 to 8, 2 to 7, 2 to 6, 2 to 5, 2 to 4, 2 to 3, 3 to 15, 3 to 14, 3 to 13, 3 to 12, 3 to 11, 3 to 10, 3 to 9, 3 to 8, 3 to 7, 3 to 6, 3 to 5, 3 to 4, 4 to 15, 4 to 14, 4 to 13, 4 to 12, 4 to 11, 4 to 10, 4 to 9, 4 to 8, 4 to 7, 4 to 6, 4 to 5, 5 to 15, 5 to 14, 5 to 13, 5 to 12, 5 to 11, 5 to 10, 5 to 9, 5 to 8, 5 to 7, 5 to 6, 6 to 15, 6 to 14, 6 to 13, 6 to 12, 6 to 11, 6 to 10, 6 to 9, 6 to 8, 6 to 7, 7 to 15, 7 to 14, 7 to 13, 7 to 12, 7 to 11, 7 to 10, 7 to 9, 7 to 8, 8 to 15, 8 to 14, 8 to 13, 8 to 12, 8 to 11, 8 to 10, 8 to 9, 9 to 15, 9 to 14, 9 to 13, 9 to 12, 9 to 11, 9 to 10, 10 to 15, 10 to 14, 10 to 13, 10 to 12, 10 to 11, 11 to 15, 11 to 14, 11 to 13, 11 to 12, 12 to 15, 12 to 14, 12 to 13, 13 to 15, 13 to 14, or 14 to 15 food products.

A subject's allergenic status (for example, whether a subject is allergic to an allergen (e.g., a food product) or a subject's tolerance threshold for an allergen (e.g., a food product)) may be determined by any suitable method known in the art. In certain embodiments, a subject's allergenic status is determined by a Double-Blind, Placebo-Controlled Food Challenge (DBPCFC). Exemplary DBPCFC methods include those described in Sampson et al. (2012) J Allergy Clin Immunol 130(6): 1260-127 and in Example 1 herein. Briefly, a food challenge is performed by feeding gradually increasing doses of a suspected allergenic food to a subject, mixed with a palatable food. The subject is evaluated after each dose for any allergic symptoms associated with the food. A corresponding placebo challenge includes placebo material given in the same number of doses and similar volume of dose material as in the suspected allergenic food. In an exemplary method, a DBPCFC includes doses of 1 mg, 3 mg, 10 mg, 30 mg, 100 mg, 300 mg, 600 mg, 1000 mg, 2000 mg, and/or 4000 mg (by protein weight) of a suspected allergenic food, administered, for example, every 15-30 minutes. In certain embodiments, a subject is not allergic to a food if they exhibit no or only mild symptoms to the food during a DBPCFC. In certain embodiments, a subject's tolerance threshold is the highest dose during the DBPCFC at which the subject exhibits no symptoms or only mild symptoms. Mild symptoms include transient or mild discomfort (<48 h) and symptoms that require no or minimal medical intervention/therapy, such as pruritus, swelling or rash, abdominal discomfort, or other transient symptoms. Additional allergic symptoms (e.g., moderate, severe, and life-threating symptoms) are described in Table 4 in Example 1 herein and in Jones et al. (2017) Journal of Allergy and Clinical Immunology 139(4): 1242-1252.e1249.

It is contemplated that a subject may, for example, be allergic to any allergen disclosed herein (including allergens described below in II. Allergens and Allergen Compositions) and similarly, that the compositions and methods disclosed herein may be used to treat an allergy to any allergen disclosed herein.

For example, in certain embodiments, a subject is allergenic to one or more foods (for example, 1, 2, 3, 4, 5, 6, 7, 8, 9, 10, 11, 12, 13, 14 or 15 foods) selected from almond, cashew, chicken's egg, codfish, cow's milk, hazelnut, peanut, pecan, pistachio, salmon, sesame seed, shrimp, soy, walnut, and wheat (e.g., wheat gluten). For example, in certain embodiments, a subject cannot tolerate 100 mg (by protein weight), as measured by DBPCFC, of one or more foods selected from almond, cashew, chicken's egg, codfish, cow's milk, hazelnut, peanut, pecan, pistachio, salmon, sesame seed, shrimp, soy, walnut, and wheat (e.g., wheat gluten). In certain embodiments, a subject is allergic to at most five foods selected from almond, cashew, chicken's egg, codfish, cow's milk, hazelnut, peanut, pecan, pistachio, salmon, sesame seed, shrimp, soy, walnut, and wheat (e.g., wheat gluten).

In certain embodiments of a contemplated method, a subject (i) has not had a severe/life-threatening episode of anaphylaxis/anaphylactic shock within 60 days of receiving the initial dose of the mixed allergen composition; (ii) does not have a history of eosinophilic esophagitis (EoE), other eosinophilic gastrointestinal disease, chronic, recurrent, or severe gastrointestinal reflux disease (GERD), symptoms of dysphagia (e.g., difficulty swallowing, food “getting stuck”), or recurrent gastrointestinal symptoms of undiagnosed etiology; (iii) does not have a history of chronic disease (other than asthma, atopic dermatitis, or allergic rhinitis) that is, or is at significant risk of becoming unstable or requiring a change in chronic therapeutic regimen; and/or (iv) has not had an active infection within 30 days of receiving the initial dose of the mixed allergen composition.

A contemplated method may, for example, include administration of an allergen composition (e.g., a mixed allergen composition) to a subject once every one, two, three, four, five, six, or seven days, or once every one, two, three, or four weeks. For example, in certain embodiments, a mixed allergen composition is administered to a subject daily (e.g., one, two, three, four, or more than four times daily).

A contemplated method may, for example, include administration of an allergen composition (e.g., a mixed allergen composition) to a subject for at least about 4, 8, 12, 16, 20, 24, 28, 32, 36, 40, 44, 48, 52, 56, 60, 80, 100, or 120 weeks, at least about 1, 2, 3, 4, 5, 6, 9, or 12 months, at least about 1, 2, 3, 4, 5 years, or from about 20 to about 120, from about 20 to about 100, from about 20 to about 80, from about 20 to about 60 from about 20 to about 40, from about 40 to about 120, from about 40 to about 100, from about 40 to about 80, from about 40 to about 60, from about 60 to about 120, from about 60 to about 100, from about 60 to about 80, from about 80 to about 120, from about 80 to about 100, or from about 100 to about 120 weeks. For example, in certain embodiments, a mixed allergen composition is administered to a subject for at least about 40 weeks.

A contemplated method may, for example, include administration of a dose of about 1 mg, 2 mg, 3 mg, 4 mg, 5 mg, 10 mg, 20 mg, 30 mg, 40 mg, 50 mg, 60 mg, 70 mg, 80 mg, 90 mg, 100 mg, 125 mg, 150 mg, 175 mg, 200 mg, 300 mg, 400 mg, 500 mg, 600 mg, 700 mg, 800 mg, 900 mg, 1000 mg, 1250 mg, 1500 mg, 1750 mg, 2000 mg, 2500 mg, 3000 mg, 4000 mg, 4500 mg, 5000 mg, 5500 mg, 6000 mg, 7000 mg, and/or 7500 mg (by total protein weight) of a mixed allergen composition. A contemplated method may, for example, include administration of a dose of from about 2.5 mg to about 7.5 mg, about 10 mg to about 30 mg, about 40 mg to about 60 mg, about 75 mg to about 125 mg, about 175 mg to about 225 mg, about 375 mg to about 425 mg, about 675 mg to about 725 mg, about 950 mg to about 1050 mg, about 1450 mg to about 1550 mg, about 2950 mg to about 3050 mg, about 4450 mg to about 4550 mg, about 5950 mg to about 6050 mg, and/or about 7450 mg to about 7550 mg (by total protein weight) of a mixed allergen composition.

A contemplated method may, for example, include administration of a dose of about 0.1 mg, 0.2 mg, 0.3 mg, 0.5 mg, 1 mg, 1.3 mg, 2 mg, 3 mg, 3.3 mg, 4 mg, 5 mg, 6 mg, 6.6 mg, 8 mg, 10 mg, 12 mg, 13.3 mg, 20 mg, 26.6 mg, 30 mg, 40 mg, 46.6 mg, 50 mg, 60 mg, 66.6 mg, 75 mg, 100 mg, 125 mg, 150 mg, 175 mg, 200 mg, 250 mg and/or 300 mg (by protein weight of each food source present) of a mixed allergen composition.

When a dose of a contemplated mixed allergen composition includes about 0.3 mg (e.g., about 0.33 mg) by protein weight of each food source present in the mixed allergen composition, the dose of the mixed allergen composition may be, for example, about 0.3 mg, 0.6 mg, 1 mg, 1.3 mg, 1.6 mg, 2 mg, 2.3 mg, 2.6 mg, 3 mg, 3.3 mg, 3.6 mg, 4 mg, 4.3 mg, 4.6 mg, or 5 mg when 1, 2, 3, 4, 5, 6, 7, 8, 9, 10, 11, 12, 13, 14, or 15 food sources, respectively, are present in the mixed allergen composition. Similarly, when a dose of a contemplated mixed allergen composition includes about 1.3 mg (e.g., about 1.33 mg) by protein weight of each food source present in the mixed allergen composition, the dose of the mixed allergen composition may be, for example, about 1.3 mg, 2.6 mg, 4 mg, 5.3 mg, 6.6 mg, 8 mg, 9.3 mg, 10.6 mg, 12 mg, 13.3 mg, 14.6 mg, 16 mg, 17.3 mg, 18.6 mg, or 20 mg when 1, 2, 3, 4, 5, 6, 7, 8, 9, 10, 11, 12, 13, 14, or 15 food sources, respectively, are present in the mixed allergen composition; when a dose of a contemplated mixed allergen composition includes about 3.3 mg (e.g., about 3.33 mg) by protein weight of each food source present in the mixed allergen composition, the dose of the mixed allergen composition may be, for example, about 3.3 mg, 6.6 mg, 10 mg, 13.3 mg, 16.6 mg, 20 mg, 23.3 mg, 26.6 mg, 30 mg, 33.3 mg, 36.6 mg, 40 mg, 43.3 mg, 46.6 mg, or 50 mg when 1, 2, 3, 4, 5, 6, 7, 8, 9, 10, 11, 12, 13, 14, or 15 food sources, respectively, are present in the mixed allergen composition; when a dose of a contemplated mixed allergen composition includes about 6.6 mg (e.g., about 6.67 mg) by protein weight of each food source present in the mixed allergen composition, the dose of the mixed allergen composition may be, for example, about 6.6 mg, 13.3 mg, 20 mg, 26.6 mg, 33.3 mg, 40 mg, 46.6 mg, 53.3 mg, 60 mg, 66.6 mg, 73.3 mg, 80 mg, 86.6 mg, 93.3 mg, or 100 mg when 1, 2, 3, 4, 5, 6, 7, 8, 9, 10, 11, 12, 13, 14, or 15 food sources, respectively, are present in the mixed allergen composition; when a dose of a contemplated mixed allergen composition includes about 13.3 mg (e.g., about 13.33 mg) by protein weight of each food source present in the mixed allergen composition, the dose of the mixed allergen composition may be, for example, about 13.3 mg, 26.6 mg, 40 mg, 53.3 mg, 66.6 mg, 80 mg, 93.3 mg, 106.6 mg, 120 mg, 133.3 mg, 146.6 mg, 160 mg, 173.3 mg, 186.6 mg, or 200 mg when 1, 2, 3, 4, 5, 6, 7, 8, 9, 10, 11, 12, 13, 14, or 15 food sources, respectively, are present in the mixed allergen composition; when a dose of a contemplated mixed allergen composition includes about 26.6 mg (e.g., about 26.67 mg) by protein weight of each food source present in the mixed allergen composition, the dose of the mixed allergen composition may be, for example, about 26.6 mg, 53.3 mg, 80 mg, 106.6 mg, 133.3 mg, 160 mg, 186.6 mg, 213.3 mg, 240 mg, 266.6 mg, 293.3 mg, 320 mg, 346.6 mg, 373.3 mg, or 400 mg when 1, 2, 3, 4, 5, 6, 7, 8, 9, 10, 11, 12, 13, 14, or 15 food sources, respectively, are present in the mixed allergen composition; when a dose of a contemplated mixed allergen composition includes about 46.6 mg (e.g., about 46.67 mg) by protein weight of each food source present in the mixed allergen composition, the dose of the mixed allergen composition may be, for example, about 46.6 mg, 93.3 mg, 140 mg, 180.6 mg, 233.3 mg, 280 mg, 326.6 mg, 373.3 mg, 420 mg, 466.6 mg, 513.3 mg, 560 mg, 606.6 mg, 653.3 mg, or 700 mg when 1, 2, 3, 4, 5, 6, 7, 8, 9, 10, 11, 12, 13, 14, or 15 food sources, respectively, are present in the mixed allergen composition; when a dose of a contemplated mixed allergen composition includes about 66.6 mg (e.g., about 66.67 mg) by protein weight of each food source present in the mixed allergen composition, the dose of the mixed allergen composition may be, for example, about 66.6 mg, 133.3 mg, 200 mg, 266.6 mg, 333.3 mg, 400 mg, 466.6 mg, 533.3 mg, 600 mg, 666.6 mg, 733.3 mg, 800 mg, 866.6 mg, 933.3 mg, or 1000 mg when 1, 2, 3, 4, 5, 6, 7, 8, 9, 10, 11, 12, 13, 14, or 15 food sources, respectively, are present in the mixed allergen composition; when a dose of a contemplated mixed allergen composition includes 100 mg by protein weight of each food source present in the mixed allergen composition, the dose of the mixed allergen composition may be, for example, about 100 mg, 200 mg, 300 mg, 400 mg, 500 mg, 600 mg, 700 mg, 800 mg, 900 mg, 1000 mg, 1100 mg, 1200 mg, 1300 mg, 1400 mg, or 1500 mg when 1, 2, 3, 4, 5, 6, 7, 8, 9, 10, 11, 12, 13, 14, or 15 food sources, respectively, are present in the mixed allergen composition; when a dose of a contemplated mixed allergen composition includes 200 mg by protein weight of each food source present in the mixed allergen composition, the dose of the mixed allergen composition may be, for example, about 200 mg, 400 mg, 600 mg, 800 mg, 1000 mg, 1200 mg, 1400 mg, 1600 mg, 1800 mg, 2000 mg, 2200 mg, 2400 mg, 2600 mg, 2800 mg, or 3000 mg when 1, 2, 3, 4, 5, 6, 7, 8, 9, 10, 11, 12, 13, 14, or 15 food sources, respectively, are present in the mixed allergen composition; and when a dose of a contemplated mixed allergen composition includes 300 mg by protein weight of each food source present in the mixed allergen composition, the dose of the mixed allergen composition may be, for example, about 300 mg, 600 mg, 900 mg, 1200 mg, 1500 mg, 1800 mg, 2100 mg, 2400 mg, 2700 mg, 3000 mg, 3300 mg, 3600 mg, 3900 mg, 4200 mg, or 4500 mg when 1, 2, 3, 4, 5, 6, 7, 8, 9, 10, 11, 12, 13, 14, or 15 food sources, respectively, are present in the mixed allergen composition.

A contemplated method may, for example, include administration of a dose of from about 0.2 mg to about 0.5 mg, about 1 mg to about 2 mg, about 3 mg to about 4 mg, about 6 mg to about 7 mg, about 12 mg to about 15 mg, about 25 mg to about 28 mg, about 45 mg to about 48 mg, about 65 mg to about 68 mg, about 90 mg to about 110 mg, about 190 mg to about 210 mg, and/or about 290 mg to about 310 mg (by total protein weight) of a mixed allergen composition.

In certain embodiments, a dose of a mixed allergen composition is increased during a course of administration (also referred to as updosing). For example, the dose of the mixed allergen composition may be increased after one, two, three, four, five, six, seven, eight, nine, ten, eleven, or twelve weeks of administration of a lower dose, or from about two to twelve weeks, two to ten weeks, two to eight weeks, two to six weeks, two to four weeks, four to twelve weeks, four to ten weeks, four to eight weeks, four to six weeks, six to twelve weeks, six to ten weeks, six to eight weeks, eight to twelve weeks, eight to ten weeks, or ten to twelve weeks of administration of a lower dose.

A dose of a mixed allergen composition may, for example, be increased to a highest tolerated dose. As used herein, the highest tolerated dose of a mixed allergen composition refers to the highest dose that elicits no or only mild symptoms in a subject. Mild symptoms include transient or mild discomfort (<48 h) and symptoms that require no or minimal medical intervention/therapy, such as pruritus, swelling or rash, abdominal discomfort, or other transient symptoms. Additional allergic symptoms (e.g., moderate, severe, and life-threating symptoms) are described in Table 4 in Example 1 herein and in Jones et al. (2017) Journal of Allergy and Clinical Immunology 139(4): 1242-1252.e1249.

In certain embodiments, a dose of a mixed allergen composition is increased from an initial dose (e.g., daily dose) of about 5 mg (by total protein weight) of the mixed allergen composition to a final dose (e.g., daily dose) that is the lower of about 4500 mg (by total protein weight) of the mixed allergen composition or a highest tolerated dose of the mixed allergen composition. For example, the dose of the mixed allergen composition may be increased from an initial dose (e.g., daily dose) of about 5 mg (by total protein weight) of the mixed allergen composition to about 20 mg, to about 50 mg, to about 100 mg, to about 200 mg, to about 400 mg, to about 700 mg, to about 1000 mg, to about 1500 mg, to about 3000 mg, to a final daily dose of about 4500 mg.

In certain embodiments, a dose of a mixed allergen composition is increased from an initial dose (e.g., daily dose) of about 5 mg (by total protein weight) of the mixed allergen composition to a final dose (e.g., daily dose) that is the lower of about 1500 mg (by total protein weight) of the mixed allergen composition or a highest tolerated dose of the mixed allergen composition. For example, the dose of the mixed allergen composition may be increased from an initial dose (e.g., daily dose) of about 5 mg (by total protein weight) of the mixed allergen composition, to about 20 mg, to about 50 mg, to about 100 mg, to about 200 mg, to about 400, mg to about 700 mg, to about 1000 mg, to a final daily dose of about 1500 mg.

In certain embodiments, a dose of a mixed allergen composition is increased from an initial dose (e.g., daily dose) of about 0.3 mg protein from each food source in the mixed allergen composition to a final dose (e.g., daily dose) that is the lower of about 300 mg of protein from each food source in the mixed allergen composition or a highest tolerated dose of the mixed allergen composition. For example, the dose of the mixed allergen composition may be increased from an initial dose (e.g., daily dose) of about 0.3 mg protein from each food source in the mixed allergen composition, to about 1.3 mg, to about 3.3 mg, to about 6.6 mg, to about 13.3 mg, to about 26.6 mg, to about 46.6 mg, to about 66.6 mg, to about 100 mg, to about 200 mg, to a final dose (e.g., daily dose) of about 300 mg protein from each food source in the mixed allergen composition.

In certain embodiments, a dose of a mixed allergen composition is increased from an initial dose (e.g., daily dose) of about 0.3 mg protein from each food source in the mixed allergen composition to a final dose (e.g., daily dose) that is the lower of about 100 mg of protein from each food source in the mixed allergen composition or a highest tolerated dose of the mixed allergen composition. For example, the dose of the mixed allergen composition may be increased from an initial dose (e.g., daily dose) of about 0.3 mg protein from each food source in the mixed allergen composition, to about 1.3 mg, to about 3.3 mg, to about 6.6 mg, to about 13.3 mg, to about 26.6 mg, to about 46.6 mg, to about 66.6 mg, to a final dose (e.g., daily dose) of about 100 mg protein from each food source in the mixed allergen composition.

In certain embodiments, a contemplated method comprises: (i) administering (e.g., orally) to a subject a dose (e.g., a daily dose) of about 5 mg (by total protein weight) of a mixed allergen composition for at least about two to six weeks; (ii) administering (e.g., orally) to the subject a dose (e.g., a daily dose) of about 20 mg of a mixed allergen composition for at least about two to six weeks; (iii) administering (e.g., orally) to the subject a dose (e.g., a daily dose) of about 50 mg of a mixed allergen composition for at least about two to six weeks; (iv) administering (e.g., orally) to the subject a dose (e.g., a daily dose) of about 100 mg of a mixed allergen composition for at least about two to six weeks; (v) administering (e.g., orally) to the subject a dose (e.g., a daily dose) of about 200 mg of a mixed allergen composition for at least about two to six weeks; (vi) administering (e.g., orally) to the subject a dose (e.g., a daily dose) of about 400 mg of a mixed allergen composition for at least about two to six weeks; (vii) administering (e.g., orally) to the subject a dose (e.g., a daily dose) of about 700 mg of a mixed allergen composition for at least about two to six weeks; (viii) administering (e.g., orally) to the subject a dose (e.g., a daily dose) of about 1000 mg of a mixed allergen composition for at least about two to six weeks; and/or (ix) administering (e.g., orally) to the subject a dose (e.g., a daily dose) of about 1500 mg of a mixed allergen composition for at least about two to six weeks. In certain embodiments, the method further comprises: (x) administering (e.g., orally) to the subject a dose (e.g., a daily dose) of about 3000 mg of a mixed allergen composition for at least about two to six weeks; and/or (xi) administering (e.g., orally) to the subject a dose (e.g., a daily dose) of about 4500 mg of a mixed allergen composition for at least about two to six weeks.

In certain embodiments, a contemplated method comprises: (i) administering (e.g., orally) to a subject a dose (e.g., a daily dose) of about 0.3 mg protein from each food source in a mixed allergen composition for at least about two to six weeks; (ii) administering (e.g., orally) to the subject a dose (e.g., a daily dose) of about 1.3 mg protein from each food source in the mixed allergen composition for at least about two to six weeks; (iii) administering (e.g., orally) to the subject a dose (e.g., a daily dose) of about 3.3 mg protein from each food source in the mixed allergen composition for at least about two to six weeks; (iv) administering (e.g., orally) to the subject a dose (e.g., a daily dose) of about 6.6 mg protein from each food source in the mixed allergen composition for at least about two to six weeks; (v) administering (e.g., orally) to the subject a dose (e.g., a daily dose) of about 13.3 mg protein from each food source in the mixed allergen composition for at least about two to six weeks; (vi) administering (e.g., orally) to the subject a dose (e.g., a daily dose) about 26.6 mg protein from each food source in the mixed allergen composition for at least about two to six weeks; (vii) administering (e.g., orally) to the subject a dose (e.g., a daily dose) of about 46.6 mg protein from each food source in the mixed allergen composition for at least about two to six weeks; (viii) administering (e.g., orally) to the subject a dose (e.g., a daily dose) of about 66.6 mg protein from each food source in the mixed allergen composition for at least about two to six weeks; and/or (ix) administering (e.g., orally) to the subject a dose (e.g., a daily dose) of about 100 mg protein from each food source in the mixed allergen composition for at least about two to six weeks. In certain embodiments, the method further comprises: (x) administering (e.g., orally) to the subject a dose (e.g., a daily dose) of about 200 mg protein from each food source in the mixed allergen composition for at least about two to six weeks; and/or (xi) administering (e.g., orally) to the subject a dose (e.g., a daily dose) of about 300 mg protein from each food source in the mixed allergen composition for at least about two to six weeks.

In certain embodiments of a contemplated method: (i) for about a first two to six weeks, a daily dose of a mixed allergen compositions is about 5 mg (by total protein weight); (ii) for about the next two to six weeks, the daily dose of the mixed allergen compositions is about 20 mg; (iii) for about the next two to six weeks, the daily dose of the mixed allergen compositions is about 50 mg; (iv) for about the next two to six weeks, the daily dose of the mixed allergen compositions is about 100 mg; (v) for about the next two to six weeks, the daily dose of the mixed allergen compositions is about 200 mg; (vi) for about the next two to six weeks, the daily dose of the mixed allergen compositions is about 400 mg; (vii) for about the next two to six weeks, the daily dose of the mixed allergen compositions is about 700 mg; (viii) for about the next two to six weeks, the daily dose of the mixed allergen compositions is about 1000 mg; (ix) for about the next two to six weeks, the daily dose of the mixed allergen compositions is about 1500 mg; (x) for about the next two to six weeks, the daily dose of the mixed allergen compositions is about 3000 mg; and (xi) for at least about the next twenty weeks, the daily dose of the mixed allergen compositions is about 4500 mg; wherein if a subject cannot tolerate a higher dose the dose is not increased and the subject is administered a highest tolerated dose for the remainder of the at least about 40 weeks.

In certain embodiments of a contemplated method: (i) for about a first two to six weeks, a daily dose of a mixed allergen compositions is about 5 mg (by total protein weight); (ii) for about the next two to six weeks, the daily dose of the mixed allergen compositions is about 20 mg; (iii) for about the next two to six weeks, the daily dose of the mixed allergen compositions is about 50 mg; (iv) for about the next two to six weeks, the daily dose of the mixed allergen compositions is about 100 mg; (v) for about the next two to six weeks, the daily dose of the mixed allergen compositions is about 200 mg; (vi) for about the next two to six weeks, the daily dose of the mixed allergen compositions is about 400 mg; (vii) for about the next two to six weeks, the daily dose of the mixed allergen compositions is about 700 mg; (viii) for about the next two to six weeks, the daily dose of the mixed allergen compositions is about 1000 mg; and (ix) for at least about the next 24 weeks, the daily dose of the mixed allergen compositions is about 1500 mg; wherein if a subject cannot tolerate a higher dose the dose is not increased and the subject is administered a highest tolerated dose for the remainder of the at least about 40 weeks.

In certain embodiments of a contemplated method: (i) for about a first two to six weeks, a daily dose of a mixed allergen compositions is about 0.3 mg protein from each food source in the mixed allergen composition; (ii) for about the next two to six weeks, the daily dose of the mixed allergen compositions is about 1.3 mg protein from each food source in the mixed allergen composition; (iii) for about the next two to six weeks, the daily dose of the mixed allergen compositions is about 3.3 mg protein from each food source in the mixed allergen composition; (iv) for about the next two to six weeks, the daily dose of the mixed allergen compositions is about 6.6 mg protein from each food source in the mixed allergen composition; (v) for about the next two to six weeks, the daily dose of the mixed allergen compositions is about 13.3 mg protein from each food source in the mixed allergen composition; (vi) for about the next two to six weeks, the daily dose of the mixed allergen compositions is about 26.6 mg protein from each food source in the mixed allergen composition; (vii) for about the next two to six weeks, the daily dose of the mixed allergen compositions is about 46.6 mg protein from each food source in the mixed allergen composition; (viii) for about the next two to six weeks, the daily dose of the mixed allergen compositions is about 66.6 mg protein from each food source in the mixed allergen composition; (ix) for about the next two to six weeks, the daily dose of the mixed allergen compositions is about 100 mg protein from each food source in the mixed allergen composition; (x) for about the next two to six weeks, the daily dose of the mixed allergen compositions is about 200 mg protein from each food source in the mixed allergen composition; and (xi) for about the next twenty weeks, the daily dose of the mixed allergen compositions is about 300 mg protein from each food source in the mixed allergen composition; wherein if a subject cannot tolerate a higher dose the dose is not increased and the subject is administered a highest tolerated dose for the remainder of the at least about 40 weeks.

In certain embodiments of a contemplated method: (i) for about a first two to six weeks, a daily dose of a mixed allergen compositions is about 0.3 mg protein from each food source in the mixed allergen composition; (ii) for about the next two to six weeks, the daily dose of the mixed allergen compositions is about 1.3 mg protein from each food source in the mixed allergen composition; (iii) for about the next two to six weeks, the daily dose of the mixed allergen compositions is about 3.3 mg protein from each food source in the mixed allergen composition; (iv) for about the next two to six weeks, the daily dose of the mixed allergen compositions is about 6.6 mg protein from each food source in the mixed allergen composition; (v) for about the next two to six weeks, the daily dose of the mixed allergen compositions is about 13.3 mg protein from each food source in the mixed allergen composition; (vi) for about the next two to six weeks, the daily dose of the mixed allergen compositions is about 26.6 mg protein from each food source in the mixed allergen composition; (vii) for about the next two to six weeks, the daily dose of the mixed allergen compositions is about 46.6 mg protein from each food source in the mixed allergen composition; (viii) for about the next two to six weeks, the daily dose of the mixed allergen compositions is about 66.6 mg protein from each food source in the mixed allergen composition; (ix) for about the next 24 weeks, the daily dose of the mixed allergen compositions is about 100 mg protein from each food source in the mixed allergen composition; wherein if a subject cannot tolerate a higher dose the dose is not increased and the subject is administered a highest tolerated dose for the remainder of the at least about 40 weeks.

In certain embodiments of a contemplated method: (i) for about a first two to six weeks, a daily dose of a mixed allergen compositions is about 5 mg (by total protein weight); (ii) for about the next two to six weeks, the daily dose of the mixed allergen compositions is about 20 mg; (iii) for about the next two to six weeks, the daily dose of the mixed allergen compositions is about 50 mg; (iv) if a subject can tolerate a daily dose of about 100 mg, for about the next two to six weeks, the daily dose of the mixed allergen compositions is about 100 mg, or, if the subject cannot tolerate a daily dose of about 100 mg, for about the next 30 to 34 weeks, the daily dose of the mixed allergen composition is about 50 mg; (v) if the subject can tolerate a daily dose of about 200 mg, for about the next two to six weeks, the daily dose of the mixed allergen compositions is about 200 mg, or, if the subject cannot tolerate a daily dose of about 200 mg, for about the next 28 to 32 weeks, the daily dose of the mixed allergen composition is about 100 mg; (vi) if the subject can tolerate a daily dose of about 400 mg, for about the next two to six weeks, the daily dose of the mixed allergen compositions is about 400 mg, or, if the subject cannot tolerate a daily dose of about 400 mg, for about the next 26 to 30 weeks, the daily dose of the mixed allergen composition is about 200 mg; (vii) if the subject can tolerate a daily dose of about 700 mg, for about the next two to six weeks, the daily dose of the mixed allergen compositions is about 700 mg, or, if the subject cannot tolerate a daily dose of about 700 mg, for about the next 24 to 28 weeks, the daily dose of the mixed allergen composition is about 400 mg; (viii) if the subject can tolerate a daily dose of about 1000 mg, for about the next two to six weeks, the daily dose of the mixed allergen compositions is about 1000 mg, or, if the subject cannot tolerate a daily dose of about 1000 mg, for about the next 22 to 26 weeks, the daily dose of the mixed allergen composition is about 700 mg; (ix) if the subject can tolerate a daily dose of about 1500 mg, for about the next two to six weeks, the daily dose of the mixed allergen compositions is about 1500 mg, or, if the subject cannot tolerate a daily dose of about 1500 mg, for about the next 20 to 24 weeks, the daily dose of the mixed allergen composition is about 1000 mg; (x) if the subject can tolerate a daily dose of about 3000 mg, for about the next two to six weeks, the daily dose of the mixed allergen compositions is about 3000 mg, or, if the subject cannot tolerate a daily dose of about 3000 mg, for about the next 18 to 22 weeks, the daily dose of the mixed allergen composition is about 1500 mg; and (xi) if the subject can tolerate a daily dose of about 4500 mg, for about the next 16 to 20 weeks, the daily dose of the mixed allergen compositions is about 4500 mg, or, if the subject cannot tolerate a daily dose of about 4500 mg, for about the next 16 to 20 weeks, the daily dose of the mixed allergen composition is about 3000 mg.

In certain embodiments of a contemplated method: (i) for about a first two to six weeks, a daily dose of a mixed allergen compositions is about 5 mg (by total protein weight); (ii) for about the next two to six weeks, the daily dose of the mixed allergen compositions is about 20 mg; (iii) for about the next two to six weeks, the daily dose of the mixed allergen compositions is about 50 mg; (iv) if a subject can tolerate a daily dose of about 100 mg, for about the next two to six weeks, the daily dose of the mixed allergen compositions is about 100 mg, or, if the subject cannot tolerate a daily dose of about 100 mg, for about the next 16 to 32 weeks, the daily dose of the mixed allergen composition is about 50 mg; (v) if the subject can tolerate a daily dose of about 200 mg, for about the next two to six weeks, the daily dose of the mixed allergen compositions is about 200 mg, or, if the subject cannot tolerate a daily dose of about 200 mg, for about the next 10 to 30 weeks, the daily dose of the mixed allergen composition is about 100 mg; (vi) if the subject can tolerate a daily dose of about 400 mg, for about the next two to six weeks, the daily dose of the mixed allergen compositions is about 400 mg, or, if the subject cannot tolerate a daily dose of about 400 mg, for about the next 4 to 28 weeks, the daily dose of the mixed allergen composition is about 200 mg; (vii) if the subject can tolerate a daily dose of about 700 mg, for about the next two to six weeks, the daily dose of the mixed allergen compositions is about 700 mg, or, if the subject cannot tolerate a daily dose of about 700 mg, for about the next 1 to 26 weeks, the daily dose of the mixed allergen composition is about 400 mg; (viii) if the subject can tolerate a daily dose of about 1000 mg, for about the next two to six weeks, the daily dose of the mixed allergen compositions is about 1000 mg, or, if the subject cannot tolerate a daily dose of about 1000 mg, for about the next 1 to 24 weeks, the daily dose of the mixed allergen composition is about 700 mg; (ix) if the subject can tolerate a daily dose of about 1500 mg, for about the next two to six weeks, the daily dose of the mixed allergen compositions is about 1500 mg, or, if the subject cannot tolerate a daily dose of about 1500 mg, for about the next 1 to 22 weeks, the daily dose of the mixed allergen composition is about 1000 mg; (x) if the subject can tolerate a daily dose of about 3000 mg, for about the next two to six weeks, the daily dose of the mixed allergen compositions is about 3000 mg, or, if the subject cannot tolerate a daily dose of about 3000 mg, for about the next 1 to 20 weeks, the daily dose of the mixed allergen composition is about 1500 mg; and (xi) if the subject can tolerate a daily dose of about 4500 mg, for about the next 1 to 20 weeks, the daily dose of the mixed allergen compositions is about 4500 mg, or, if the subject cannot tolerate a daily dose of about 4500 mg, for about the next 1 to 18 weeks, the daily dose of the mixed allergen composition is about 3000 mg.

In certain embodiments of a contemplated method: (i) for about a first two to six weeks, a daily dose of a mixed allergen compositions is about 5 mg (by total protein weight); (ii) for about the next two to six weeks, the daily dose of the mixed allergen compositions is about 20 mg; (iii) for about the next two to six weeks, the daily dose of the mixed allergen compositions is about 50 mg; (iv) if a subject can tolerate a daily dose of about 100 mg, for about the next two to six weeks, the daily dose of the mixed allergen compositions is about 100 mg, or, if the subject cannot tolerate a daily dose of about 100 mg, for about the next 30 to 34 weeks, the daily dose of the mixed allergen composition is about 50 mg; (v) if the subject can tolerate a daily dose of about 200 mg, for about the next two to six weeks, the daily dose of the mixed allergen compositions is about 200 mg, or, if the subject cannot tolerate a daily dose of about 200 mg, for about the next 28 to 32 weeks, the daily dose of the mixed allergen composition is about 100 mg; (vi) if the subject can tolerate a daily dose of about 400 mg, for about the next two to six weeks, the daily dose of the mixed allergen compositions is about 400 mg, or, if the subject cannot tolerate a daily dose of about 400 mg, for about the next 26 to 30 weeks, the daily dose of the mixed allergen composition is about 200 mg; (vii) if the subject can tolerate a daily dose of about 700 mg, for about the next two to six weeks, the daily dose of the mixed allergen compositions is about 700 mg, or, if the subject cannot tolerate a daily dose of about 700 mg, for about the next 24 to 28 weeks, the daily dose of the mixed allergen composition is about 400 mg; (viii) if the subject can tolerate a daily dose of about 1000 mg, for about the next two to six weeks, the daily dose of the mixed allergen compositions is about 1000 mg, or, if the subject cannot tolerate a daily dose of about 1000 mg, for about the next 22 to 26 weeks, the daily dose of the mixed allergen composition is about 700 mg; (ix) if the subject can tolerate a daily dose of about 1500 mg, for about the next 20 to 24 weeks, the daily dose of the mixed allergen compositions is about 1500 mg, or, if the subject cannot tolerate a daily dose of about 1500 mg, for about the next 20 to 24 weeks, the daily dose of the mixed allergen composition is about 1000 mg.

In certain embodiments of a contemplated method: (i) for about a first two to six weeks, a daily dose of a mixed allergen compositions is about 5 mg (by total protein weight); (ii) for about the next two to six weeks, the daily dose of the mixed allergen compositions is about 20 mg; (iii) for about the next two to six weeks, the daily dose of the mixed allergen compositions is about 50 mg; (iv) if a subject can tolerate a daily dose of about 100 mg, for about the next two to six weeks, the daily dose of the mixed allergen compositions is about 100 mg, or, if the subject cannot tolerate a daily dose of about 100 mg, for about the next 16 to 32 weeks, the daily dose of the mixed allergen composition is about 50 mg; (v) if the subject can tolerate a daily dose of about 200 mg, for about the next two to six weeks, the daily dose of the mixed allergen compositions is about 200 mg, or, if the subject cannot tolerate a daily dose of about 200 mg, for about the next 10 to 30 weeks, the daily dose of the mixed allergen composition is about 100 mg; (vi) if the subject can tolerate a daily dose of about 400 mg, for about the next two to six weeks, the daily dose of the mixed allergen compositions is about 400 mg, or, if the subject cannot tolerate a daily dose of about 400 mg, for about the next 4 to 28 weeks, the daily dose of the mixed allergen composition is about 200 mg; (vii) if the subject can tolerate a daily dose of about 700 mg, for about the next two to six weeks, the daily dose of the mixed allergen compositions is about 700 mg, or, if the subject cannot tolerate a daily dose of about 700 mg, for about the next 1 to 26 weeks, the daily dose of the mixed allergen composition is about 400 mg; (viii) if the subject can tolerate a daily dose of about 1000 mg, for about the next two to six weeks, the daily dose of the mixed allergen compositions is about 1000 mg, or, if the subject cannot tolerate a daily dose of about 1000 mg, for about the next 1 to 24 weeks, the daily dose of the mixed allergen composition is about 700 mg; and (ix) if the subject can tolerate a daily dose of about 1500 mg, for about the next 1 to 24 weeks, the daily dose of the mixed allergen compositions is about 1500 mg, or, if the subject cannot tolerate a daily dose of about 1500 mg, for about the next 1 to 22 weeks, the daily dose of the mixed allergen composition is about 1000 mg.

In certain embodiments of a contemplated method: (i) for about a first two to six weeks, a daily dose of a mixed allergen compositions is about 0.3 mg protein from each food source in the mixed allergen composition; (ii) for about the next two to six weeks, the daily dose of the mixed allergen compositions is about 1.3 mg protein from each food source in the mixed allergen composition; (iii) for about the next two to six weeks, the daily dose of the mixed allergen compositions is about 3.3 mg protein from each food source in the mixed allergen composition; (iv) if a subject can tolerate a daily dose of about 6.6 mg protein from each food source in the mixed allergen composition, for about the next two to six weeks, the daily dose of the mixed allergen compositions is about 6.6 mg protein from each food source in the mixed allergen composition, or, if the subject cannot tolerate a daily dose of about 6.6 mg protein from each food source in the mixed allergen composition, for about the next 30 to 34 weeks, the daily dose of the mixed allergen composition is about 3.3 mg protein from each food source in the mixed allergen composition; (v) if the subject can tolerate a daily dose of about 13.3 mg protein from each food source in the mixed allergen composition, for about the next two to six weeks, the daily dose of the mixed allergen compositions is about 13.3 mg protein from each food source in the mixed allergen composition, or, if the subject cannot tolerate a daily dose of about 13.3 mg protein from each food source in the mixed allergen composition, for about the next 28 to 32 weeks, the daily dose of the mixed allergen composition is about 6.6 mg protein from each food source in the mixed allergen composition; (vi) if the subject can tolerate a daily dose of about 26.6 mg protein from each food source in the mixed allergen composition, for about the next two to six weeks, the daily dose of the mixed allergen compositions is about 26.6 mg protein from each food source in the mixed allergen composition, or, if the subject cannot tolerate a daily dose of about 26.6 mg protein from each food source in the mixed allergen composition, for about the next 26 to 30 weeks, the daily dose of the mixed allergen composition is about 13.3 mg protein from each food source in the mixed allergen composition; (vii) if the subject can tolerate a daily dose of about 46.6 mg protein from each food source in the mixed allergen composition, for about the next two to six weeks, the daily dose of the mixed allergen compositions is about 46.6 mg protein from each food source in the mixed allergen composition, or, if the subject cannot tolerate a daily dose of about 46.6 mg protein from each food source in the mixed allergen composition, for about the next 24 to 28 weeks, the daily dose of the mixed allergen composition is about 26.6 mg protein from each food source in the mixed allergen composition; (viii) if the subject can tolerate a daily dose of about 66.6 mg protein from each food source in the mixed allergen composition, for about the next two to six weeks, the daily dose of the mixed allergen compositions is about 66.6 mg protein from each food source in the mixed allergen composition, or, if the subject cannot tolerate a daily dose of about 66.6 mg protein from each food source in the mixed allergen composition, for about the next 22 to 26 weeks, the daily dose of the mixed allergen composition is about 46.6 mg protein from each food source in the mixed allergen composition; (ix) if the subject can tolerate a daily dose of about 100 mg protein from each food source in the mixed allergen composition, for about the next two to six weeks, the daily dose of the mixed allergen compositions is about 100 mg protein from each food source in the mixed allergen composition, or, if the subject cannot tolerate a daily dose of about 100 mg protein from each food source in the mixed allergen composition, for about the next 20 to 24 weeks, the daily dose of the mixed allergen composition is about 66.6 mg protein from each food source in the mixed allergen composition; (x) if the subject can tolerate a daily dose of about 200 mg protein from each food source in the mixed allergen composition, for about the next two to six weeks, the daily dose of the mixed allergen compositions is about 200 mg protein from each food source in the mixed allergen composition, or, if the subject cannot tolerate a daily dose of about 200 mg protein from each food source in the mixed allergen composition, for about the next 18 to 22 weeks, the daily dose of the mixed allergen composition is about 100 mg protein from each food source in the mixed allergen composition; and (xi) if the subject can tolerate a daily dose of about 300 mg protein from each food source in the mixed allergen composition, for about the next 16 to 20 weeks, the daily dose of the mixed allergen compositions is about 300 mg protein from each food source in the mixed allergen composition, or, if the subject cannot tolerate a daily dose of about 300 mg protein from each food source in the mixed allergen composition, for about the next 16 to 20 weeks, the daily dose of the mixed allergen composition is about 200 mg protein from each food source in the mixed allergen composition.

In certain embodiments of a contemplated method: (i) for about a first two to six weeks, a daily dose of a mixed allergen compositions is about 0.3 mg protein from each food source in the mixed allergen composition; (ii) for about the next two to six weeks, the daily dose of the mixed allergen compositions is about 1.3 mg protein from each food source in the mixed allergen composition; (iii) for about the next two to six weeks, the daily dose of the mixed allergen compositions is about 3.3 mg protein from each food source in the mixed allergen composition; (iv) if a subject can tolerate a daily dose of about 6.6 mg protein from each food source in the mixed allergen composition, for about the next two to six weeks, the daily dose of the mixed allergen compositions is about 6.6 mg protein from each food source in the mixed allergen composition, or, if the subject cannot tolerate a daily dose of about 6.6 mg protein from each food source in the mixed allergen composition, for about the next 16 to 32 weeks, the daily dose of the mixed allergen composition is about 3.3 mg protein from each food source in the mixed allergen composition; (v) if the subject can tolerate a daily dose of about 13.3 mg protein from each food source in the mixed allergen composition, for about the next two to six weeks, the daily dose of the mixed allergen compositions is about 13.3 mg protein from each food source in the mixed allergen composition, or, if the subject cannot tolerate a daily dose of about 13.3 mg protein from each food source in the mixed allergen composition, for about the next 10 to 30 weeks, the daily dose of the mixed allergen composition is about 6.6 mg protein from each food source in the mixed allergen composition; (vi) if the subject can tolerate a daily dose of about 26.6 mg protein from each food source in the mixed allergen composition, for about the next two to six weeks, the daily dose of the mixed allergen compositions is about 26.6 mg protein from each food source in the mixed allergen composition, or, if the subject cannot tolerate a daily dose of about 26.6 mg protein from each food source in the mixed allergen composition, for about the next 4 to 28 weeks, the daily dose of the mixed allergen composition is about 13.3 mg protein from each food source in the mixed allergen composition; (vii) if the subject can tolerate a daily dose of about 46.6 mg protein from each food source in the mixed allergen composition, for about the next two to six weeks, the daily dose of the mixed allergen compositions is about 46.6 mg protein from each food source in the mixed allergen composition, or, if the subject cannot tolerate a daily dose of about 46.6 mg protein from each food source in the mixed allergen composition, for about the next 1 to 26 weeks, the daily dose of the mixed allergen composition is about 26.6 mg protein from each food source in the mixed allergen composition; (viii) if the subject can tolerate a daily dose of about 66.6 mg protein from each food source in the mixed allergen composition, for about the next two to six weeks, the daily dose of the mixed allergen compositions is about 66.6 mg protein from each food source in the mixed allergen composition, or, if the subject cannot tolerate a daily dose of about 66.6 mg protein from each food source in the mixed allergen composition, for about the next 1 to 24 weeks, the daily dose of the mixed allergen composition is about 46.6 mg protein from each food source in the mixed allergen composition; (ix) if the subject can tolerate a daily dose of about 100 mg protein from each food source in the mixed allergen composition, for about the next two to six weeks, the daily dose of the mixed allergen compositions is about 100 mg protein from each food source in the mixed allergen composition, or, if the subject cannot tolerate a daily dose of about 100 mg protein from each food source in the mixed allergen composition, for about the next 1 to 22 weeks, the daily dose of the mixed allergen composition is about 66.6 mg protein from each food source in the mixed allergen composition; (x) if the subject can tolerate a daily dose of about 200 mg protein from each food source in the mixed allergen composition, for about the next two to six weeks, the daily dose of the mixed allergen compositions is about 200 mg protein from each food source in the mixed allergen composition, or, if the subject cannot tolerate a daily dose of about 200 mg protein from each food source in the mixed allergen composition, for about the next 1 to 20 weeks, the daily dose of the mixed allergen composition is about 100 mg protein from each food source in the mixed allergen composition; and (xi) if the subject can tolerate a daily dose of about 300 mg protein from each food source in the mixed allergen composition, for about the next 1 to 20 weeks, the daily dose of the mixed allergen compositions is about 300 mg protein from each food source in the mixed allergen composition, or, if the subject cannot tolerate a daily dose of about 300 mg protein from each food source in the mixed allergen composition, for about the next 1 to 18 weeks, the daily dose of the mixed allergen composition is about 200 mg protein from each food source in the mixed allergen composition.

In certain embodiments of a contemplated method: (i) for about a first two to six weeks, a daily dose of a mixed allergen compositions is about 0.3 mg protein from each food source in the mixed allergen composition; (ii) for about the next two to six weeks, the daily dose of the mixed allergen compositions is about 1.3 mg protein from each food source in the mixed allergen composition; (iii) for about the next two to six weeks, the daily dose of the mixed allergen compositions is about 3.3 mg protein from each food source in the mixed allergen composition; (iv) if a subject can tolerate a daily dose of about 6.6 mg protein from each food source in the mixed allergen composition, for about the next two to six weeks, the daily dose of the mixed allergen compositions is about 6.6 mg protein from each food source in the mixed allergen composition, or, if the subject cannot tolerate a daily dose of about 6.6 mg protein from each food source in the mixed allergen composition, for about the next 30 to 34 weeks, the daily dose of the mixed allergen composition is about 3.3 mg protein from each food source in the mixed allergen composition; (v) if the subject can tolerate a daily dose of about 13.3 mg protein from each food source in the mixed allergen composition, for about the next two to six weeks, the daily dose of the mixed allergen compositions is about 13.3 mg protein from each food source in the mixed allergen composition, or, if the subject cannot tolerate a daily dose of about 13.3 mg protein from each food source in the mixed allergen composition, for about the next 28 to 32 weeks, the daily dose of the mixed allergen composition is about 6.6 mg protein from each food source in the mixed allergen composition; (vi) if the subject can tolerate a daily dose of about 26.6 mg protein from each food source in the mixed allergen composition, for about the next two to six weeks, the daily dose of the mixed allergen compositions is about 26.6 mg protein from each food source in the mixed allergen composition, or, if the subject cannot tolerate a daily dose of about 26.6 mg protein from each food source in the mixed allergen composition, for about the next 26 to 30 weeks, the daily dose of the mixed allergen composition is about 13.3 mg protein from each food source in the mixed allergen composition; (vii) if the subject can tolerate a daily dose of about 46.6 mg protein from each food source in the mixed allergen composition, for about the next two to six weeks, the daily dose of the mixed allergen compositions is about 46.6 mg protein from each food source in the mixed allergen composition, or, if the subject cannot tolerate a daily dose of about 46.6 mg protein from each food source in the mixed allergen composition, for about the next 24 to 28 weeks, the daily dose of the mixed allergen composition is about 26.6 mg protein from each food source in the mixed allergen composition; (viii) if the subject can tolerate a daily dose of about 66.6 mg protein from each food source in the mixed allergen composition, for about the next two to six weeks, the daily dose of the mixed allergen compositions is about 66.6 mg protein from each food source in the mixed allergen composition, or, if the subject cannot tolerate a daily dose of about 66.6 mg protein from each food source in the mixed allergen composition, for about the next 22 to 26 weeks, the daily dose of the mixed allergen composition is about 46.6 mg protein from each food source in the mixed allergen composition; and (ix) if the subject can tolerate a daily dose of about 100 mg protein from each food source in the mixed allergen composition, for about the next 20 to 24 weeks, the daily dose of the mixed allergen compositions is about 100 mg protein from each food source in the mixed allergen composition, or, if the subject cannot tolerate a daily dose of about 100 mg protein from each food source in the mixed allergen composition, for about the next 20 to 24 weeks, the daily dose of the mixed allergen composition is about 66.6 mg protein from each food source in the mixed allergen composition.

In certain embodiments of a contemplated method: (i) for about a first two to six weeks, a daily dose of a mixed allergen compositions is about 0.3 mg protein from each food source in the mixed allergen composition; (ii) for about the next two to six weeks, the daily dose of the mixed allergen compositions is about 1.3 mg protein from each food source in the mixed allergen composition; (iii) for about the next two to six weeks, the daily dose of the mixed allergen compositions is about 3.3 mg protein from each food source in the mixed allergen composition; (iv) if a subject can tolerate a daily dose of about 6.6 mg protein from each food source in the mixed allergen composition, for about the next two to six weeks, the daily dose of the mixed allergen compositions is about 6.6 mg protein from each food source in the mixed allergen composition, or, if the subject cannot tolerate a daily dose of about 6.6 mg protein from each food source in the mixed allergen composition, for about the next 16 to 32 weeks, the daily dose of the mixed allergen composition is about 3.3 mg protein from each food source in the mixed allergen composition; (v) if the subject can tolerate a daily dose of about 13.3 mg protein from each food source in the mixed allergen composition, for about the next two to six weeks, the daily dose of the mixed allergen compositions is about 13.3 mg protein from each food source in the mixed allergen composition, or, if the subject cannot tolerate a daily dose of about 13.3 mg protein from each food source in the mixed allergen composition, for about the next 10 to 30 weeks, the daily dose of the mixed allergen composition is about 6.6 mg protein from each food source in the mixed allergen composition; (vi) if the subject can tolerate a daily dose of about 26.6 mg protein from each food source in the mixed allergen composition, for about the next two to six weeks, the daily dose of the mixed allergen compositions is about 26.6 mg protein from each food source in the mixed allergen composition, or, if the subject cannot tolerate a daily dose of about 26.6 mg protein from each food source in the mixed allergen composition, for about the next 4 to 28 weeks, the daily dose of the mixed allergen composition is about 13.3 mg protein from each food source in the mixed allergen composition; (vii) if the subject can tolerate a daily dose of about 46.6 mg protein from each food source in the mixed allergen composition, for about the next two to six weeks, the daily dose of the mixed allergen compositions is about 46.6 mg protein from each food source in the mixed allergen composition, or, if the subject cannot tolerate a daily dose of about 46.6 mg protein from each food source in the mixed allergen composition, for about the next 1 to 26 weeks, the daily dose of the mixed allergen composition is about 26.6 mg protein from each food source in the mixed allergen composition; (viii) if the subject can tolerate a daily dose of about 66.6 mg protein from each food source in the mixed allergen composition, for about the next two to six weeks, the daily dose of the mixed allergen compositions is about 66.6 mg protein from each food source in the mixed allergen composition, or, if the subject cannot tolerate a daily dose of about 66.6 mg protein from each food source in the mixed allergen composition, for about the next 1 to 24 weeks, the daily dose of the mixed allergen composition is about 46.6 mg protein from each food source in the mixed allergen composition; and (ix) if the subject can tolerate a daily dose of about 100 mg protein from each food source in the mixed allergen composition, for about the next 1 to 24 weeks, the daily dose of the mixed allergen compositions is about 100 mg protein from each food source in the mixed allergen composition, or, if the subject cannot tolerate a daily dose of about 100 mg protein from each food source in the mixed allergen composition, for about the next 1 to 22 weeks, the daily dose of the mixed allergen composition is about 66.6 mg protein from each food source in the mixed allergen composition.

It will be appreciated that the exact dosage of a pharmaceutical unit, pharmaceutical composition, or allergen composition is chosen by an individual physician in view of the patient to be treated, in general, dosage and administration are adjusted to provide an effective amount of the agent to the patient being treated. As used herein, the “effective amount” refers to the amount necessary to elicit a beneficial or desired biological response. An effective amount can be administered in one or more administrations, applications or dosages and is not intended to be limited to a particular formulation or administration route. As will be appreciated by those of ordinary skill in this art, the effective amount of a pharmaceutical unit, pharmaceutical composition, or allergen composition may vary depending on such factors as the desired biological endpoint, the drug to be delivered, the target tissue, the route of administration, etc. Additional factors which may be taken into account include the severity of the disease state; age, weight and gender of the patient being treated; diet, time and frequency of administration; drug combinations; reaction sensitivities; and tolerance/response to therapy.

In certain embodiments, any contemplated method or dosing regimen described herein for an allergen composition further comprises one or more moratorium durations wherein the dosage (e.g., daily dose) of the allergen composition is either decreased or remains the same (i.e., a moratorium on dosage increase). For example, in some embodiments, the dosage (e.g., daily dose) remains the same for one or more weeks. In some embodiments, the daily dose is lowered to a previous dosage amount (e.g., previous daily dose), such as the immediately preceding dosage or an earlier dosage. In some embodiments, the daily dose is lowered back to the initial dosage.

In some embodiments, said one or more moratorium durations is implemented due to one or more occurrences. For example, in some embodiments, a moratorium duration is implemented due to the subject being unable to tolerate a higher dosage. In some embodiments, the moratorium duration is implemented due to the subject having an underlying illness or other illness (e.g., a cold, flu, etc.) that may i) make it difficult to determine if symptoms experienced by the subject are due to the illness or increased dosage of the allergen composition; and/or ii) place the subject at a higher risk of an adverse reaction due to the increased dosage and illness, and in some cases, medications taken by the subject. In some embodiments, the moratorium duration is implemented so as to reduce or prevent a risk of the subject administering an intolerable dosage during a specified period of time. For example, the subject may have an important event occurring, such as for example, a wedding, school finals, a sporting event, etc., wherein it would be desirable to avoid any risk of hampering the subject's ability to enjoy and/or participate in the given event (due to for example, a higher dosage of the allergen composition). Other examples include, for example, the subject going on vacation, attending an event that may result in ingestion of high level of alcohol and/or other recreational drugs, etc.

In some embodiments, each moratorium duration of the one or more moratorium durations lasts one or more days, one or more weeks, at least about 2 weeks, etc. In some embodiments, the length of each moratorium duration is dependent on the type of occurrence applicable to the subject (e.g., wedding, vacation, school finals). In some embodiments, the need and/or length for a moratorium duration is determined by a subject (e.g., patient), by a physician, and/or by a family member or caregiver of the patient.

The methods and compositions described herein can be used alone or in combination with other therapeutic agents and/or modalities. The term administered “in combination,” as used herein, is understood to mean that two (or more) different treatments are delivered to the subject during the course of the subject's affliction with the disorder, such that the effects of the treatments on the patient overlap at a point in time. In certain embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In certain embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In certain embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered. In certain embodiments, a side effect of a first and/or second treatment is reduced because of combined administration.

A contemplated method may, for example, increase the tolerance of a subject for an allergic food (e.g., increase the amount of food that the subject can consume with no or only mild symptoms). The amount of an allergic food that the subject can tolerate may be measured, for example, by a double-blind, placebo-controlled food challenge (DBPCFC), as described above. As a DBPCFC includes feeding of multiple, gradually increasing doses of a suspected allergenic food to a subject, tolerance may be measured by the highest individual dose of food consumed during a DBPCFC with no or only mild symptoms, or the total, cumulative amount of food consumed during a DBPCFC with no or only mild symptoms. A contemplated method may, for example, increase a highest amount of an allergenic food that a subject can tolerate in a DBPCFC from less than or equal to about 100 mg (by protein weight; e.g., about 1 mg, 3 mg, 10 mg, 30 mg, or 100 mg) to greater than or equal to about 300 mg (by protein weight, e.g., about 300 mg, 600 mg, 1000 mg, 2000 mg, 4000 mg, or greater). A contemplated method may, for example, increase the total amount of an allergenic food that a subject can tolerate in a DBPCFC from less than or equal to about 150 mg (by protein weight; e.g., about 1 mg, 3 mg, 10 mg, 30 mg, 100 mg, or 144 mg) to greater than or equal to about 400 mg (by protein weight, e.g., about 400 mg, 444 mg, 1000 mg, 1044 mg, 2000 mg, 2044 mg, 4000 mg, 4044 mg, 8000 mg, 8044 mg, or greater).

Each administration as part of a contemplated method may be performed at any appropriate location. For example, in certain embodiments of a contemplated method, each dose of the mixed allergen composition is administered to the subject in a clinic. Alternatively, for example, when a subject has a mild to moderate food allergy, one or more doses of the mixed allergen composition may be administered to the subject elsewhere. For example, one or more doses of the mixed allergen composition may be administered at the subject's home, and/or via telemedicine. Similarly, up-dosing of the mixed allergen composition may be performed in a clinic or elsewhere. For example, a dose of the mixed allergen composition may be increased at the subject's home, and/or via telemedicine.

In some embodiments, each administration of an allergenic composition, as part of a contemplated method, may be age specific. For example, in some embodiments, the rate of increase in dosage amount is tailored according to specific age groups. In some embodiments, children under the age of 1 year, 2 years, 5 years, 10 years, 12 years, and/or 18 years may be specified a specific dose rate and dosage increase rate. In some embodiments, elderly persons over the age of 55 years, 60 years, 65 years, 70 years, 75 years, 80 years, 85 years, and/or 90 years may be specified with a specified dose rate and dosage increase rate.

In some embodiments, each administration of an allergenic composition, as part of a contemplated method, may be specific to one or more characteristics of a subject. For example, in some embodiments, the rate of increase in dosage amount is tailored according to a specific underlying health condition of the patients, such as a cardiovascular disease, diabetes, etc.

### II. Allergens and Allergen Compositions

As used herein, an “allergenic composition” is understood to mean a composition that includes one or more different allergens or allergenic components. Allergenic compositions are understood to include “mixed allergen compositions” that include two or more different allergens, where any two given allergens are different if they are distinct from each other, e.g., they are compounds described by different chemical formula or compositions described by different components and/or amounts thereof. The number of different allergens in a composition may vary, as desired. In certain embodiments, a mixed allergen composition comprises 2 or more different allergens, such as 3 or more different allergens, 4 or more different allergens, 5 or more different allergens, 6 or more different allergens, 7 or more different allergens, 8 or more different allergens, 9 or more different allergens, 10 or more different allergens, 15 or more different allergens, 20 or more different allergens, 25 or more different allergens, 30 or more different allergens, 40 or more different allergens, 50 or more different allergens, 75 or more different allergens, 100 or more different allergens, 200 or more different allergens, 300 or more different allergens, 400 or more different allergens, 500 or more different allergens, 600 or more different allergens, 700 or more different allergens, 800 or more different allergens, 900 or more different allergens, 1000 or more different allergens, 2000 or more different allergens, 4000 or more different allergens, 6000 or more different allergens, 8000 or more different allergens, or 10000 or more different allergens. In certain embodiments, a mixed allergen composition comprises 10000 or fewer different allergens, 8000 or fewer different allergens, 6000 or fewer different allergens, 4000 or fewer different allergens, 2000 or fewer different allergens, 1000 or fewer different allergens, 900 or fewer different allergens, 800 or fewer different allergens, 700 or fewer different allergens, 600 or fewer different allergens, 500 or fewer different allergens, 400 or fewer different allergens, 300 or fewer different allergens, 200 or fewer different allergens, 100 or fewer different allergens, such as 75 or fewer different allergens, 50 or fewer different allergens, 25 or fewer different allergens, 15 or fewer different allergens, or 10 or fewer different allergens. In certain embodiments, a composition may include 2 to 20 different allergens, 2 to 100 different allergens, or 2 to 1000 different allergens. In further embodiments, a composition may comprise 6 to 20 different allergens. In certain embodiments, a composition may consist essentially of 6 to 20 different protein allergens.

Allergens present in an allergenic composition may vary, where in some instances an allergen present in the composition is one that induces an allergy in a susceptible subject. Allergens include any antigen, or active derivative thereof, that elicits a specific IgE response. Antigens include any substance that can stimulate the production of antibodies and can combine specifically with them. Allergens may have little or no intrinsic toxicity by themselves, but cause a pathological condition due to their ability to elicit an IgE-associated immune response, and, upon subsequent exposure, due to their ability to elicit IgE- and/or T cell-dependent hypersensitivity reactions. As such, an allergen includes any substance which is capable of stimulating a typical hypersensitivity reaction in atopic subjects. Allergens that may be present in a given allergenic composition include any substance found in a variety of different sources, e.g., foods, drugs, perfume, plants, the environment or biological systems (e.g., prokaryotic or eukaryotic cells or viruses), as well as chemical allergens.

It is appreciated that reference to an allergen or an allergenic composition may each include a plurality of different proteins as found in the naturally occurring allergen (either raw or cooked). For example, a provided food product may include a peanut allergen composition (which would include substantially all peanut proteins present in e.g., defatted peanuts, ground peanuts, etc.). As used herein the phrase “complete allergen” refers to all possible antigenic components of a given food product.

Allergens of interest include nut allergens. Nut allergens are allergens that include one or more compounds found in nuts, e.g., dry fruits that include an edible kernel or meat enclosed in a woody or leathery shell. Nut allergens of interest include, e.g. peanut allergens, (e.g., rAra h 1, rAra h 2, rAra h 3, rAra h 8 PR-10, rAra h 9 LTP, or peanut complete allergen), brazil nut allergens (e.g., rBer e 1, or brazil nut complete allergen), hazelnut or filbert allergens (e.g., rCor a 1 PR-10, rCor a 8 LTP, nCor a 9, rCor a 14, or hazel nut complete allergen), walnut allergens (e.g., rJug r 1, rJug r 3 LTP, or walnut complete allergen), cashew allergens (e.g., cashew component allergens, or cashew complete allergen), pistachio allergens (e.g., pistachio component allergens, or pistachio complete allergen), pecan allergens (e.g., pecan component allergens, or pecan complete allergen), almond allergens (e.g., almond component allergens, or almond complete allergen), or tree nut component package allergens (e.g., one or more allergens from e.g., cashew nut, walnut, hazelnut, or brazil nut).

Allergens of interest include animal allergens. Animal allergens are allergens that include one or more compounds found in animals, including both vertebrates and invertebrates. Vertebrate animal allergens that may be present in an allergenic composition include avian allergens (e.g., egg allergens, e.g., nGal d 1 Ovomucoid, n Gal d 2 Ovalbumin, nGal d 3 Conalbumin, or egg white complete allergen), mammalian allergens (e.g. milk allergens, e.g., nBos d 4 alpha-lactalbumin, nBos d 5 beta-lactoglobulin, nBos d 8 Casein, nBos d Lactoferrin, or milk complete allergen), or fish allergens (e.g., rCyp c 1, rGad c 1, cod complete allergen, white fish allergens, or pink fish allergens). Invertebrate animal allergens that may be present in an allergenic composition include crustacean allergens (e.g., shrimp allergens, e.g., rPen a 1 tropomyosin, or shrimp complete allergen), or insect allergens (e.g., bee sting venom allergen, wasp sting venom allergen, or mosquito bite allergen).

Allergens of interest include non-nut plant allergens, i.e., plant allergens that are not nut allergens. Plant allergens are allergens that include one or more compounds found in plants. Plant allergens of interest include wheat allergens (e.g., rTri a 19 Omega-5 Gliadin, gliadin wheat, rTri a 14 LTP, or wheat (e.g., wheat gluten) complete allergen), fruit allergens (e.g., kiwi allergens, e.g., rAct d 8 PR-10, or kiwi complete allergen), vegetable allergens (e.g., carrot allergens, or celery allergens, e.g., rApi g 1.01 PR-10, rPhl p 12, or celery complete allergen), CCD MUXF3 from Bromelain, legume allergens (e.g., soy allergens or chickpea allergens, e.g., rGly m 4 PR-10, nGly m 5 Beta-conglycinin, nGly m 6 Glycinin, or soy complete allergen), stone fruit allergens, e.g., f419, f420, f421, f95, f242, o214 rPru p 1 PR-10, rPru p 3 LTP, or stone fruit primary complete allergen), oat allergens (e.g., oat component allergens, or oat complete allergen), or seed allergens (e.g., sesame allergens, e.g., sesame seed component allergens, or sesame seed complete allergen).

Additional types of allergens that may be present in an allergenic unit, component or composition include, e.g., non-food animal allergens (e.g., cats or dog fur and dander, cockroach calyx, dust mite excretion), drug allergens (penicillin, sulfonamides, salicylates, local anesthetics), mold spore allergens, latex allergens, metal allergens, or plant pollen allergens (e.g. from grass, e.g., ryegrass or timothy-grass, from weeds, e.g., ragweed, plantago, nettle, Artemisia, vulgaris, Chenopodium album, sorrel, or e.g., from trees, e.g., birch alder, hazel, hombeam, aesculus, willow, poplar, platanus, tilia, or olea).

A contemplated mixed allergen composition may comprise, for example, an allergen from at least 6, 7, 8, 9, 10, 11, 12, 13, 14, or 15 different food sources. In certain embodiments, a mixed allergen composition comprises an almond, cashew, cod, egg, hazelnut, milk, peanut, pecan, pistachio, salmon, sesame, shrimp, soy, walnut, or wheat (e.g., wheat gluten) allergen, or any combination thereof. In certain embodiments, a mixed allergen composition comprises an almond, cashew, cod, egg, hazelnut, milk, peanut, pecan, pistachio, salmon, sesame, shrimp, soy, walnut, and wheat (e.g., wheat gluten) allergen. In certain embodiments, a mixed allergen composition comprises almond protein, cashew protein, cod powder, dried eggs, hazelnut flour, milk protein, peanut flour, pecan flour, pistachio flour, salmon powder, sesame flour, shrimp powder, soy protein, walnut flour, or wheat (e.g., wheat gluten), or any combination thereof. In certain embodiments, a mixed allergen composition comprises almond protein, cashew protein, cod powder, dried eggs, hazelnut flour, milk protein, peanut flour, pecan flour, pistachio flour, salmon powder, sesame flour, shrimp powder, soy protein, walnut flour, and wheat (e.g., wheat gluten).

The amount of a given allergen in an allergenic composition, as desired. In certain embodiments, the amount of a given allergen ranges from about 1 to about 15,000 mg, about 5 to about 15,000 mg, about 10 to about 10,000 mg, about 15 to about 5,000 mg, about 10 to about 100 mg, or about 15 to about 100 mg. In certain embodiments, the amount of a given allergen is about 30 mg. The weight percentage of a given allergen in an allergenic unit, component, or may vary, as desired. In certain embodiments, the weight percentage of a given allergen in an allergenic unit, component, or composition ranges from about 0.1 to about 99.9 wt. %, about 0.1 to about 15 wt. %, about 15 to about 99.9 wt. %, or about 25 to about 65 wt. %. The amount of a given allergen in an allergenic unit, component, or composition may be recited by total mass, or by protein mass, which may vary for a given allergen depending upon the weight percentage of protein in that allergen.

In certain embodiments, if more than one allergen is present in an allergenic composition, e.g., in a mixed allergen composition, any two of the mixed allergens, or all of the mixed allergens, are present in equal parts, e.g., in a 1:1 ratio, such that each allergen is present in the composition in equal weight.

In certain embodiments, one or more of the doses of the mixed allergen composition comprises: about 0.3 mg, about 1.3 mg, about 6.6 mg, about 13.3 mg, about 26.6 mg, about 46.6 mg, about 66.6 mg about 100 mg, about 200 mg, or about 300 mg of protein from each food source present in the mixed allergen composition; and/or about 5 mg, about 20 mg, about 50 mg, about 100 mg, about 200 mg, about 400 mg, about 700 mg, about 1000 mg, about 1500 mg, about 3000 mg, or about 4000 mg of total food protein.

A disclosed allergenic composition may include one or more vitamins, as desired. Vitamins that may be present include. e.g., vitamin A (e.g., in an amount ranging from 1 to 35,000 IU), vitamin C (e.g., in an amount ranging from about 1 to about 1,000 mg), vitamin D (e.g., in an amount ranging from about 1 to about 4,000 IU, i.e., from about 0.025 to about 100 mcg), vitamin E (e.g., in an amount ranging from about 1 to about 450 IU), vitamin K (e.g., in an amount ranging from about 1 to about 250 mcg), vitamin B-1 (thiamin; e.g., in amount ranging from about 1 to about 15 mg), vitamin B-2 (riboflavin; e.g., in an amount ranging from about 1 to about 17 mg), vitamin B-3 (niacin; e.g., in an amount ranging from about 1 to about 200 mg), vitamin B-5 (pantothenic acid; e.g., in an amount ranging from about 1 to about 100 mg), vitamin B-6 (pyridoxine; e.g., in an amount ranging from about 1 to about 30 mg), vitamin B-9 (folic acid; e.g., in an amount ranging from about 1 to about 4,000 mcg), vitamin B-12 (cobalamin; e.g., in an amount ranging from about 1 to about 250 mcg), vitamin H (biotin; e.g., in an amount ranging from about 1 to about 1,000 mcg) and combinations thereof. In certain embodiments, an allergenic unit, component, or composition comprises vitamin D. In certain embodiments, an allergenic unit, component, or composition comprises about 400 IU, i.e., about 10 mcg, of vitamin D.

It is appreciated that a disclosed allergen or protein may be in the form of a flour, powder, meal, paste, etc. In some embodiments, a disclosed unit or composition comprises about 30 mg protein by weight of each specific protein or allergen contained therein, e.g. about 30 mg by protein weight of an allergenic component each selected as described herein from peanut, tree nut, cow's milk, soy, egg, fish and shellfish.

Also provided are physiological acceptable compositions that include a disclosed allergenic composition and a physiologically acceptable delivery vehicle. Disclosed allergenic units, components, or compositions can be incorporated into a variety of formulations for administration to a subject. More particularly, a disclosed allergenic unit, component, or composition can be formulated into a physiological acceptable composition by combination with appropriate, physiologically acceptable carriers or diluents, for example, a vegetable oil.

In certain embodiments, a disclosed allergenic unit, component, or composition is designed for oral administration, for example, as foods, tablets, troches, lozenges, aqueous or oily suspensions, dispersible powders or granules, emulsions, hard or soft capsules, or syrups or elixirs, gums, etc. Compositions intended for oral use may be prepared according to any convenient protocol for the manufacture of pharmaceutical compositions and such compositions may contain one or more agents selected from the group consisting of sweetening agents, flavoring agents, coloring agents and preserving agents in order to provide palatable preparations. In some embodiments, an allergenic composition (as described herein) is administered orally to the subject using a syringe.

Also provided are allergenic compositions that are food products. Food products of interest include a disclosed allergenic unit, component, or composition in combination with a food delivery vehicle. By food delivery vehicle is meant a delivery vehicle that is a nourishing substance that is eaten, drunk, or otherwise taken into the body to sustain life, provide energy, promote growth, etc. Examples of food delivery vehicles or food products of interest include, but are not limited to: baby or infant formula, baby food (e.g., pureed food suitable for infant or toddler consumption), chips, cookies, breads, spreads, creams, yogurts, liquid drinks, chocolate containing products, candies, ice creams, cereals, coffees, pureed food products, etc. In certain embodiments, the composition is a food supplement.

In certain embodiments, a disclosed allergenic composition may include a bulking agent. Exemplary bulking agents include maltodextrin, sucrose, trehalose, trehalose dihydrate, mannitol, lactose, dextrose, fructose, raffinose, or any combination thereof. In certain embodiments, the bulking agent comprises maltodextrin, or sucrose, or a combination thereof. In certain embodiments, the bulking agent comprises maltodextrin and sucrose at a weight ratio of about 3:1.

In certain embodiments, an allergenic unit, component, or composition may include excipients, e.g., a food safe oil, a polysaccharide (e.g., gellan gum), flavoring, and a food safe salt (e.g., dipotassium phosphate).

In certain embodiments an allergenic composition is an aqueous suspension containing a disclosed allergenic component in admixture with excipients suitable for the manufacture of aqueous suspensions. Such excipients may include suspending agents, for example sodium carboxymethyl-cellulose, methylcellulose, hydroxy-propylmethycellulose, sodium alginate, polyvinyl-pyrrolidone, gum tragacanth and gum acacia; dispersing or wetting agents such as a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose, saccharin or aspartame.

In certain embodiments an allergenic composition is an oily suspension containing an allergenic composition suspended in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Disclosed allergenic compositions may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soybean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, preservative and flavoring and coloring agents. A disclosed composition may be in the form of a sterile aqueous or oleagenous suspension. This suspension may be formulated according to the known art using those suitable dispersing or wetting agents and suspending agents which have been mentioned above. The sterile preparation may also be a sterile solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

### III. Kits

In addition, the disclosure provides kits comprising mixed allergen compositions, for example, mixed allergen compositions disclosed herein. The kits may include one more dosages of a mixed allergen composition and/or one or more containers including a mixed allergen composition.

Exemplary containers include bottles, dispensers, packages, compartinents, cups, boxes, or any other type of container into which a mixed allergen composition may be placed. A contaier can include indicia on its surface, for example, a word, a phrase, anu abbreviation, a picture, or a synboi. A container can include a pre-determined amount of a mixed allergen composition. Contemplated kits may also include a container housing components of the kit in close confinement for cornmercial sale. Such containers nay include injection or blow-inolded plastic containers limo which desired botles, dispensers, or packages are retanied. A kit can also include instructions for employing components of the kit as well the use of any other compositions, compounds, agents, active ingredients, or objects not included in the kit. Instructions may include variations that canu be inplemented. The instructions can include an explanation of how to apply, use, and maitaini the products or compositions, for example.

A contemplated kit comprises: (i) at least about 14 five mg (by total protein weight) doses of a mixed allergen composition; (ii) at least about 14 20 mg doses of the mixed allergen composition; (iii) at least about 14 50 mg doses of the mixed allergen composition; (iv) at least about 14 100 mg doses of the mixed allergen composition; (v) at least about 14 200 mg doses of the mixed allergen composition; (vi) at least about 14 400 mg doses of the mixed allergen composition; (vii) at least about 14 700 mg doses of the mixed allergen composition; (viii) at least about 14 1000 mg doses of the mixed allergen composition; and/or (ix) at least about 14 1500 mg doses of the mixed allergen composition. In certain embodiments, a contemplated kit further comprises: (x) at least about 14 3000 mg doses of the mixed allergen composition; (xi) at least about 14 4500 mg doses of the mixed allergen composition; (xii) at least about 14 6000 mg doses of the mixed allergen composition; and/or (xiii) at least about 14 7500 mg doses of the mixed allergen composition.

A contemplated kit comprises: (i) at least about 21 five mg (by total protein weight) doses of a mixed allergen composition; (ii) at least about 21 20 mg doses of the mixed allergen composition; (iii) at least about 21 50 mg doses of the mixed allergen composition; (iv) at least about 21 100 mg doses of the mixed allergen composition; (v) at least about 21 200 mg doses of the mixed allergen composition; (vi) at least about 21 400 mg doses of the mixed allergen composition; (vii) at least about 21 700 mg doses of the mixed allergen composition; (viii) at least about 21 1000 mg doses of the mixed allergen composition; and/or (ix) at least about 21 1500 mg doses of the mixed allergen composition. In certain embodiments, a contemplated kit further comprises: (x) at least about 21 3000 mg doses of the mixed allergen composition; (xi) at least about 21 4500 mg doses of the mixed allergen composition; (xii) at least about 21 6000 mg doses of the mixed allergen composition; and/or (xiii) at least about 21 7500 mg doses of the mixed allergen composition.

A contemplated kit comprises: (i) at least about 30 five mg (by total protein weight) doses of a mixed allergen composition; (ii) at least about 30 20 mg doses of the mixed allergen composition; (iii) at least about 30 50 mg doses of the mixed allergen composition; (iv) at least about 30 100 mg doses of the mixed allergen composition; (v) at least about 30 200 mg doses of the mixed allergen composition; (vi) at least about 30 400 mg doses of the mixed allergen composition; (vii) at least about 30 700 mg doses of the mixed allergen composition; (viii) at least about 30 1000 mg doses of the mixed allergen composition; and/or (ix) at least about 30 1500 mg doses of the mixed allergen composition. In certain embodiments, a contemplated kit further comprises: (x) at least about 30 3000 mg doses of the mixed allergen composition; (xi) at least about 30 4500 mg doses of the mixed allergen composition; (xii) at least about 30 6000 mg doses of the mixed allergen composition; and/or (xiii) at least about 30 7500 mg doses of the mixed allergen composition.

A contemplated kit comprises: (i) at least about 5 to 35 five mg (by total protein weight) doses of a mixed allergen composition; (ii) at least about 5 to 35 20 mg doses of the mixed allergen composition; (iii) at least about 5 to 35 50 mg doses of the mixed allergen composition; (iv) at least about 5 to 35 100 mg doses of the mixed allergen composition; (v) at least about 5 to 35 200 mg doses of the mixed allergen composition; (vi) at least about 5 to 35 400 mg doses of the mixed allergen composition; (vii) at least about 5 to 35 700 mg doses of the mixed allergen composition; (viii) at least about 5 to 35 1000 mg doses of the mixed allergen composition; and/or (ix) at least about 5 to 35 1500 mg doses of the mixed allergen composition. In certain embodiments, a contemplated kit further comprises: (x) at least about 5 to 35 3000 mg doses of the mixed allergen composition; (xi) at least about 5 to 35 4500 mg doses of the mixed allergen composition; (xii) at least about 5 to 35 6000 mg doses of the mixed allergen composition; and/or (xiii) at least about 5 to 35 7500 mg doses of the mixed allergen composition.

It is understood that when a kit includes a dose of a mixed allergen composition, that dose may, for example, be included in a single container, or the contents of one or more containers may be combined to arrive at the desired dose. Similarly, a dose, or a portion of a dose, may, for example, initially be included in a primary container, and one or more primary containers may, for example, then be included in an additional, or secondary, container.

In some embodiments, each kit comprises one or more containers comprising one or more dosage amounts of an allergen composition (as described herein). In some embodiments, each container includes one or more packages of an allergen composition of a specific dosage amount. Accordingly, in some embodiments, a kit comprises a plurality of containers, wherein each container comprises one or more packages of a specific dosage amount. The plurality of containers thus include different dosage amount of the allergen composition, for use with a method as described herein.

A contemplated kit comprises: at least 70 containers (e.g., cups) each comprising about 5 mg (by total protein weight) of a mixed allergen composition; at least 42 containers (e.g., cups) each comprising about 50 mg (by total protein weight) of the mixed allergen composition; at least 56 containers (e.g., cups) each comprising about 200 mg (by total protein weight) of the mixed allergen composition; at least 42 containers (e.g., cups) each comprising about 500 mg (by total protein weight) of the mixed allergen composition; and at least 14 containers (e.g., cups) each comprising about 1500 mg (by total protein weight) of the mixed allergen composition. In certain embodiments, a contemplated kit further comprises at least an additional 70 containers (e.g., cups) each comprising about 1500 mg (by total protein weight) of the mixed allergen composition.

A contemplated kit comprises: at least 105 containers (e.g., cups) each comprising about 5 mg (by total protein weight) of a mixed allergen composition; at least 63 containers (e.g., cups) each comprising about 50 mg (by total protein weight) of the mixed allergen composition; at least 84 containers (e.g., cups) each comprising about 200 mg (by total protein weight) of the mixed allergen composition; at least 63 containers (e.g., cups) each comprising about 500 mg (by total protein weight) of the mixed allergen composition; and at least 21 containers (e.g., cups) each comprising about 1500 mg (by total protein weight) of the mixed allergen composition. In certain embodiments, a contemplated kit further comprises at least an additional 105 containers (e.g., cups) each comprising about 1500 mg (by total protein weight) of the mixed allergen composition.

A contemplated kit comprises: at least 14 containers (e.g., cups) each comprising about 5 mg (by total protein weight) of a mixed allergen composition.

A contemplated kit comprises: at least 21 containers (e.g., cups) each comprising about 5 mg (by total protein weight) of a mixed allergen composition.

A contemplated kit comprises: comprising at least 56 containers (e.g., cups) each comprising about 5 mg (by total protein weight) of a mixed allergen composition. In certain embodiments, a contemplated kit comprises 4 secondary containers (e.g., boxes), which each comprise at least 14 primary containers (e.g., cups) each comprising about 5 mg (by total protein weight) of the mixed allergen composition.

A contemplated kit comprises: at least 84 containers (e.g., cups) each comprising about 5 mg (by total protein weight) of a mixed allergen composition. In certain embodiments, a contemplated kit comprises 4 secondary containers (e.g., boxes), which each comprise at least 21 primary containers (e.g., cups) each comprising about 5 mg (by total protein weight) of the mixed allergen composition.

A contemplated kit comprises: at least 14 containers (e.g., cups) each comprising about 50 mg (by total protein weight) of a mixed allergen composition.

A contemplated kit comprises: at least 21 containers (e.g., cups) each comprising about 50 mg (by total protein weight) of a mixed allergen composition.

A contemplated kit comprises: at least 28 containers (e.g., cups) each comprising about 50 mg (by total protein weight) of a mixed allergen composition. In certain embodiments, a contemplated kit comprises 2 secondary containers (e.g., boxes), which each comprise at least 14 primary containers (e.g., cups) each comprising about 50 mg (by total protein weight) of the mixed allergen composition.

A contemplated kit comprises: at least 42 containers (e.g., cups) each comprising about 50 mg (by total protein weight) of a mixed allergen composition. In certain embodiments, a contemplated kit comprises 2 secondary containers (e.g., boxes), which each comprise at least 21 primary containers (e.g., cups) each comprising about 50 mg (by total protein weight) of the mixed allergen composition.

A contemplated kit comprises: at least 14 containers (e.g., cups) each comprising about 200 mg (by total protein weight) of a mixed allergen composition.

A contemplated kit comprises: at least 21 containers (e.g., cups) each comprising about 200 mg (by total protein weight) of a mixed allergen composition.

A contemplated kit comprises: at least 28 containers (e.g., cups) each comprising about 200 mg (by total protein weight) of a mixed allergen composition. In certain embodiments, a contemplated kit comprises 2 secondary containers (e.g., boxes), which each comprise at least 14 primary containers (e.g., cups) each comprising about 200 mg (by total protein weight) of the mixed allergen composition.

A contemplated kit comprises: at least 42 containers (e.g., cups) each comprising about 200 mg (by total protein weight) of a mixed allergen composition. In certain embodiments, a contemplated kit comprises 2 secondary containers (e.g., boxes), which each comprise at least 21 primary containers (e.g., cups) each comprising about 200 mg (by total protein weight) of the mixed allergen composition.

A contemplated kit comprises: at least 14 containers (e.g., cups) each comprising about 200 mg (by total protein weight) of a mixed allergen composition and at least 14 containers (e.g., cups) each comprising about 500 mg (by total protein weight) of the mixed allergen composition. In certain embodiments, a contemplated kit comprises a first secondary container (e.g., a box) which comprises at least 14 primary containers (e.g., cups) each comprising about 200 mg (by total protein weight) of the mixed allergen composition; and a second secondary container (e.g., a box) which comprises at least 14 primary containers (e.g., cups) each comprising about 500 mg (by total protein weight) of the mixed allergen composition.

A contemplated kit comprises: at least 21 containers (e.g., cups) each comprising about 200 mg (by total protein weight) of a mixed allergen composition and at least 21 containers (e.g., cups) each comprising about 500 mg (by total protein weight) of the mixed allergen composition. In certain embodiments, a contemplated kit comprises a first secondary container (e.g., a box) which comprises at least 21 primary containers (e.g., cups) each comprising about 200 mg (by total protein weight) of the mixed allergen composition; and a second secondary container (e.g., a box) which comprises at least 21 primary containers (e.g., cups) each comprising about 500 mg (by total protein weight) of the mixed allergen composition.

A contemplated kit comprises: at least 28 containers (e.g., cups) each comprising about 500 mg (by total protein weight) of a mixed allergen composition. In certain embodiments, a contemplated kit comprises 2 secondary containers (e.g., boxes), which each comprise at least 14 primary containers (e.g., cups) each comprising about 500 mg (by total protein weight) of the mixed allergen composition.

A contemplated kit comprises: at least 42 containers (e.g., cups) each comprising about 500 mg (by total protein weight) of a mixed allergen composition. In certain embodiments, a contemplated kit comprises 2 secondary containers (e.g., boxes), which each comprise at least 21 primary containers (e.g., cups) each comprising about 500 mg (by total protein weight) of the mixed allergen composition.

A contemplated kit comprises: at least 14 containers (e.g., cups) each comprising about 1500 mg (by total protein weight) of a mixed allergen composition.

A contemplated kit comprises: at least 21 containers (e.g., cups) each comprising about 1500 mg (by total protein weight) of a mixed allergen composition.

A contemplated kit comprises: at least 28 containers (e.g., cups) each comprising about 1500 mg (by total protein weight) of a mixed allergen composition. In certain embodiments, a contemplated kit comprises 2 secondary containers (e.g., boxes), which each comprise at least 14 primary containers (e.g., cups) each comprising about 1500 mg (by total protein weight) of the mixed allergen composition.

A contemplated kit comprises: at least 42 containers (e.g., cups) each comprising about 1500 mg (by total protein weight) of a mixed allergen composition. In certain embodiments, a contemplated kit comprises 2 secondary containers (e.g., boxes), which each comprise at least 21 primary containers (e.g., cups) each comprising about 1500 mg (by total protein weight) of the mixed allergen composition.

A contemplated kit comprises: at least 42 containers (e.g., cups) each comprising about 1500 mg (by total protein weight) of a mixed allergen composition. In certain embodiments, a contemplated kit comprises 3 secondary containers (e.g., boxes), which each comprise at least 14 primary containers (e.g., cups) each comprising about 1500 mg (by total protein weight) of the mixed allergen composition.

A contemplated kit comprises: at least 63 containers (e.g., cups) each comprising about 1500 mg (by total protein weight) of a mixed allergen composition. In certain embodiments, a contemplated kit comprises 2 secondary containers (e.g., boxes), which each comprise at least 21 primary containers (e.g., cups) each comprising about 1500 mg (by total protein weight) of the mixed allergen composition.

A contemplated kit comprises: at least 14 containers each comprising about 5 mg (by total protein weight) of a mixed allergen composition. In another aspect, provided herein is a kit comprising at least 14 containers each comprising about 50 mg (by total protein weight) of a mixed allergen composition. In another aspect, provided herein is a kit comprising at least 14 containers each comprising about 200 mg (by total protein weight) of a mixed allergen composition. In another aspect, provided herein is a kit comprising at least 14 containers each comprising about 500 mg (by total protein weight) of a mixed allergen composition. In another aspect, provided herein is a kit comprising at least 14 containers each comprising about 1500 mg (by total protein weight) of a mixed allergen composition. In another aspect, provided herein is a kit comprising at least 21 containers each comprising about 5 mg (by total protein weight) of a mixed allergen composition. In another aspect, provided herein is a kit comprising at least 21 containers each comprising about 50 mg (by total protein weight) of a mixed allergen composition. In another aspect, provided herein is a kit comprising at least 21 containers each comprising about 200 mg (by total protein weight) of a mixed allergen composition. In another aspect, provided herein is a kit comprising at least 21 containers each comprising about 500 mg (by total protein weight) of a mixed allergen composition. In another aspect, provided herein is a kit comprising at least 21 containers each comprising about 1500 mg (by total protein weight) of a mixed allergen composition.

Throughout the description, where compositions are described as having, including, or comprising specific components, or where processes and methods are described as having, including, or comprising specific steps, it is contemplated that, additionally, there are compositions of the present disclosure that consist essentially of, or consist of, the recited components, and that there are processes and methods according to the present disclosure that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present disclosure, whether explicit or implicit herein. For example, where reference is made to a particular compound, that compound can be used in various embodiments of compositions of the present disclosure and/or in methods of the present disclosure, unless otherwise understood from the context. In other words, within this application, embodiments have been described and depicted in a way that enables a clear and concise application to be written and drawn, but it is intended and will be appreciated that embodiments may be variously combined or separated without parting from the present teachings and disclosure. For example, it will be appreciated that all features described and depicted herein can be applicable to all aspects of the disclosure described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

It should be understood that the order of steps or order for performing certain actions is immaterial so long as the present disclosure remains operable. Moreover, two or more steps or actions may be conducted simultaneously.

The use of any and all examples, or exemplary language herein, for example, “such as” or “including,” is intended merely to illustrate better the present disclosure and does not pose a limitation on the scope of the disclosure unless claimed. No language in the specification should be construed as indicating any non-claimed element as essential to the practice of the present disclosure.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure. Where the use of the term “about” is before a quantitative value, the present disclosure also includes the specific quantitative value itself, unless specifically stated otherwise.

The foregoing examples are presented herein for illustrative purposes only, and should not be construed as limiting in any way

### EXAMPLES

### Example 1

This example describes the protocol for a phase 1/2, randomized, double-blind, placebo-controlled study evaluating the efficacy and safety of formulation A for oral immunotherapy in food-allergic children and adults.

### Background

Food allergy (FA) is a common and serious condition that affects children and adults and is commonly associated with severe reactions, including life-threatening anaphylaxis. Allergy to peanuts, tree nuts, fish, or shellfish is most commonly associated with fatal and near-fatal food-induced anaphylaxis. Published reports suggest that the prevalence of FA has been rising, and it is now estimated to affect up to 10% of the worldwide population.

Multi-allergic subjects are becoming increasingly common in the United States. Recent data show that approximately 30% to 60% of food-allergic patients were allergic to multiple foods In nationwide epidemiological surveys of US adults and children, subjects who are reactive to foods other than peanuts also show high rates of allergy to multiple foods. In both adults and children, approximately 90% or more of subjects allergic to 1 tree nut (e.g., walnuts, almonds, hazelnuts, pecans, cashews, or pistachios) were allergic to multiple foods, and more than 60% of subjects with milk, egg, wheat, soy, finfish, shrimp, or peanut allergies were allergic to multiple foods. In a separate study, about one-half of multi-allergic subjects were found to have unique combinations of FAs. Allergies to multiple foods increase the risk of anaphylaxis (due to accidental ingestion), increase anxiety, and worsen the disabling nature of FA for patients and their families, with significant impact on quality of life. Children with multiple FAs are also more likely to experience an allergic reaction (0.7 to 3.4 reactions per year) compared with children with single FA (0.2 reactions per year). The current options in the management of the majority of foods that trigger FA include OIT for specific foods (e.g., PALFORZIA® for peanut allergy), dietary avoidance of the allergenic food, and education of the patient/family on acute management of an allergic reaction. The burden of avoidance and constant fear of accidental exposure can negatively affect the health-related quality of life (HRQoL) of both patients and their families. Despite efforts toward strict food allergen avoidance, accidental exposure continues to be a major concern in FA because allergic responses can be triggered after ingestion of milligram quantities of food allergen protein.

An approach that has shown promising results is allergen-specific immunotherapy, a therapy that entails administration of increasing amounts of an allergen to individuals with immunoglobulin E (IgE)-mediated FA to raise the threshold and decrease the severity of allergic responses to the allergenic food. These allergen-based immunotherapies include sublingual immunotherapy, epicutaneous immunotherapy, and oral immunotherapy (OIT).

OIT for FA using individual food allergens such as peanuts, milk, and eggs has been widely studied in recent years and has demonstrated that OIT can effectively desensitize a majority of patients to a food allergen.

### Study Drug

Formulation A is an oral formulation mixture of 15 individual food sources from commercially available food flours containing allergenic proteins (almond, cashew, chicken's egg, codfish, cow's milk, hazelnut, peanut, pecan, pistachio, salmon, sesame seed, shrimp, soy, walnut, and wheat) with excipients, aroma and flavor maskers, and coloring agents. Formulation A contains allergenic proteins that are responsible for approximately 90% of food allergies (FAs) in the United States.

Each dose is formulated to contain equal parts by protein weight of each of the 15 individual foods in formulation A. The total protein value assigned by the food manufacturer is used to make this calculation and may vary slightly from lot to lot of raw materials. Furthermore, because salmon powder raw material contains added cod powder (cod is added to salmon as a processing aid due to the lower fat content of cod), an appropriate adjustment to the amount of each of these 2 products is made to ensure that both cod and salmon proteins are equivalently represented in the final drug products. Variations in the amount of protein are compensated for by adjusting the amount of excipient in the final blend to maintain the same unit weight from batch to batch.

There are 2 formulations of Formulation A, 10% protein by weight and 25% protein by weight blends. The use of 2 different formulations allows for a step-wise dose escalation approach beginning with a dose low enough to minimize adverse reactions while higher doses can be delivered in volumes that are reasonable for consumption by pediatric study participants.

The 2 formulations of Formulation A are prepared at unit dose strength(s) including 5, 50, 200, 500, and 1500 mg total protein per unit, as described in Table 1.

TABLE 1

| | | Nominal | | |
|---|---|---|---|---|
| | | Protein | | |
| | Nominal | (mg) for | | Total |
| | Protein | Each Drug | Excipients | Weight |
| Formulation | (mg/unit) | Substance | (mg/unit) | (mg/unit) |
| Formulation A | 5 | 0.3 | 45 | 50 |
| 10% protein | | | | |
| Formulation A | 50 | 3.3 | 450 | 500 |
| 10% protein | | | | |
| Formulation A | 200 | 13.3 | 1800 | 2000 |
| 10% protein | | | | |
| Formulation A | 500 | 33.3 | 1500 | 2000 |
| 25% protein | | | | |
| Formulation A | 1500 | 100 | 4500 | 6000 |
| 25% protein | | | | |

Formulation A is administered orally, daily. Study drug is to be mixed with semisolid food, such as applesauce, yogurt, pudding, or other palatable, age-appropriate food to which the subject is not allergic, and is to be consumed (eaten) promptly in its entirety and on a daily basis.

### Study Rationale

Formulation A is being developed as an oral immunotherapy (OIT) for the treatment of FA triggered by 1 or more of the food sources found in formulation A in children and adults. The goal of OIT for FA is to induce a state of clinically meaningful desensitization to food proteins, defined as the absence of moderate or severe allergic reaction following ingestion of small, but potentially dangerous, amounts of food protein. For example, in peanut-allergic subjects, increasing the baseline threshold before immunotherapy from 100 mg or less of peanut protein to 300 mg or more of peanut protein post-immunotherapy reduces the risk of experiencing an allergic reaction by more than 95% for 4 common food product categories that may contain trace levels of peanut residue. This level of risk reduction is highly clinically relevant. Further increase in the threshold to 1000 mg has an additional quantitative benefit in risk reduction.

It is anticipated that, in this study, subjects with double-blind, placebo-controlled food challenge (DBPCFC)-confirmed qualifying FA to at least 1 and no more than 5 food sources in formulation A will be gradually desensitized to these Reactive Food through controlled exposure and updosing. It is also anticipated that the consumption of Non-reactive Food in formulation A will not lead to development of new clinical FA.

### Objectives and Endpoints

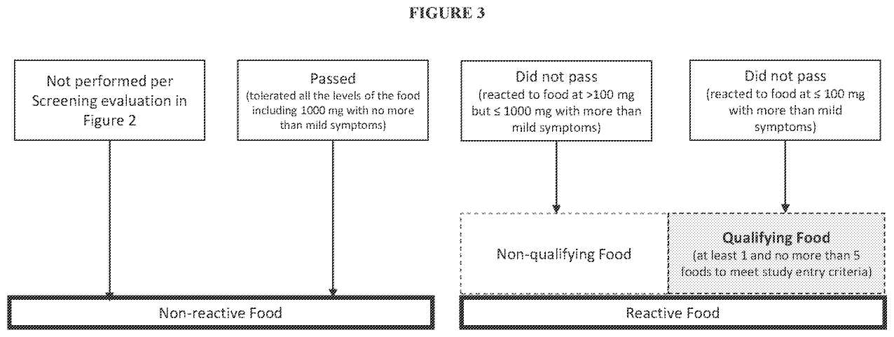

The primary objective of the study is to evaluate the efficacy of formulation A as assessed by an increased threshold for clinical reactivity to at least one Qualifying Food (see FIG. 3) for which the eliciting dose was ≤100 mg at Screening. The primary endpoint of the study is the proportion of subjects who tolerate the 600-mg level of a single Qualifying Food with no more than mild symptoms at the Exit double-blind, placebo controlled food challenge (DBPCFC).

The secondary objective of the study is to evaluate the efficacy of formulation A as assessed by an increased threshold to one or more Qualifying Food (for which the eliciting dose was ≤100 mg at Screening). The secondary endpoints of the study include: the proportion of subjects who tolerate the 1000-mg level of a single Qualifying Food with no more than mild symptoms at the Exit DBPCFC; the proportion of subjects with >1 qualifying FA who tolerate the 600-mg level of each of 2 or more Qualifying Foods with no more than mild symptoms at the Exit DBPCFC; and the proportion of subjects with >1 qualifying FA who tolerate the 1000-mg level of each of 2 or more Qualifying Foods with no more than mild symptoms at the Exit DBPCFC.

The study will also evaluate the safety and tolerability of formulation A in subjects with FA including the incidence of adverse events (AEs) and serious adverse events (SAEs).

An exploratory objective of the study is to evaluate whether Non-qualifying Reactive Foods (eliciting dose >100 mg) respond to treatment with formulation A and to evaluate impact of treatment on changes in FA-associated biomarkers. Related exploratory endpoints include change from baseline in eliciting dose of individual Reactive Food at the Exit DBPCFC with no or mild symptoms, changes from baseline in biomarkers, including, but not limited to, specific immunoglobulin E (sIgE), of all foods contained in formulation A, and change from baseline in skin prick test (SPT) for all foods contained in formulation A. An additional exploratory objective of the study is to evaluate the impact of formulation A on patient-reported outcome (PRO) measures, including FA-related quality of life, risk of accidental exposure, quality-of-life questionnaires, and treatment satisfaction. Related exploratory endpoints include quality-of-life changes over time using the following: Food Allergy Quality of Life Questionnaire (FAQLQ); Accidental exposure questionnaire; Immunotherapy-related quality-of-life (IQoL) questionnaire; 9-Item Treatment Satisfaction Questionnaire for Medication (TSQM 9); and Food Allergy Quality of Life-Parental Burden (FAQL-PB) questionnaire.

An additional exploratory objective of the study is to evaluate the development of confirmed new FAs to Non-reactive Foods contained in formulation A. A related exploratory endpoint is the proportion of subjects taking formulation A vs. placebo who develop confirmed new FAs to previously Non-reactive Foods at Screening as demonstrated by the Exit DBPCFC

An additional objective of the study is exploration of additional safety endpoints. Related exploratory endpoints include: incidence of AEs that lead to withdrawal from the study and/or discontinuation of study drug; incidence and shifts of clinically significant abnormalities in laboratory tests, vital signs, and spirometry forced expiratory volume in the first second (FEV1)/peak expiratory flow rate (PEFR); and frequency of allergic reaction AEs during treatment normalized for duration of treatment.

Additional measures to be investigated include: the maximum severity of symptoms observed at the Screening and/or Exit DBPCFC to each Qualifying Food at any dose; the maximum tolerated dose (MTD) achieved with no or mild symptoms at Screening and/or Exit DBPCFC for each Qualifying Food; the change in maximum tolerated dose (MTD) from Screening to Exit DBPCFC for each Non-qualifying Reactive Food; the average maintenance dose and time to reach the maintenance dose; the percentage of visits with successful updosing target attainment; accidental exposures (e.g., the proportion of subjects with >1 accidental allergen exposure); and the use of epinephrine as a rescue medication overall at the Exit DBPCFC and in comparison to its use at the Screening DBPCFC.

### Overall Design

The study is a Phase 1/2, randomized, double-blind, placebo-controlled study of the efficacy and safety of formulation A in subjects who are allergic to 1 or more of the 15 food sources included in formulation A. Approximately 72 subjects will be enrolled, including at least 60 subjects aged ≥4 to <18 years at study entry and up to 12 adult subjects (≥18 to ≤55 years old).

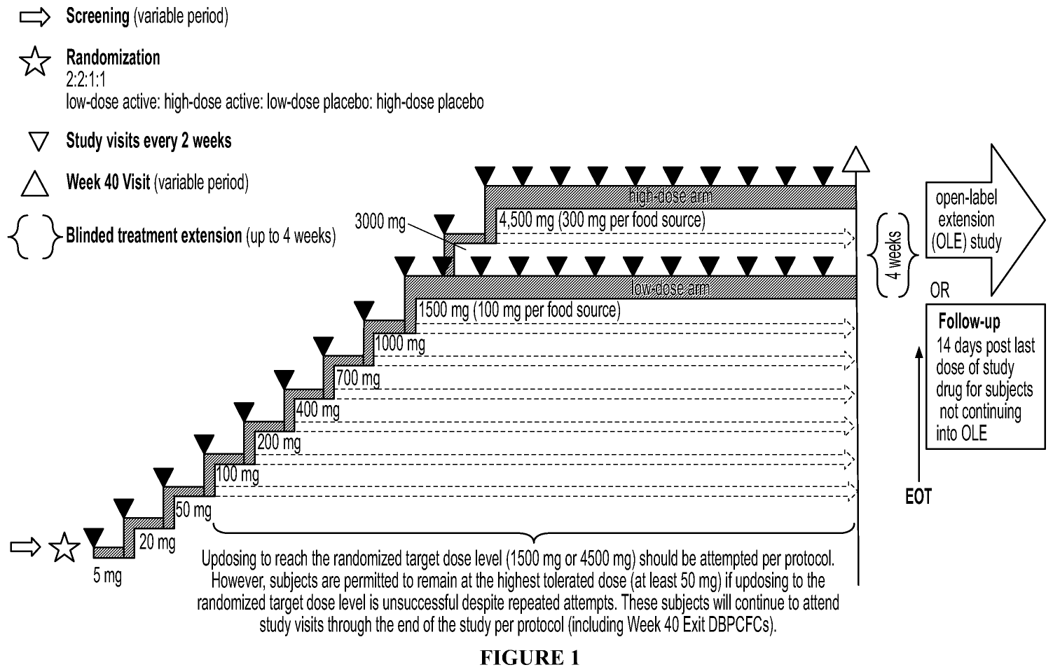

The study will include a screening period followed by a double-blind, placebo-controlled treatment period and follow up period (only for subjects not continuing to the open-label extension [OLE]) (FIG. 1). The treatment period will include both updosing and maintenance portions.

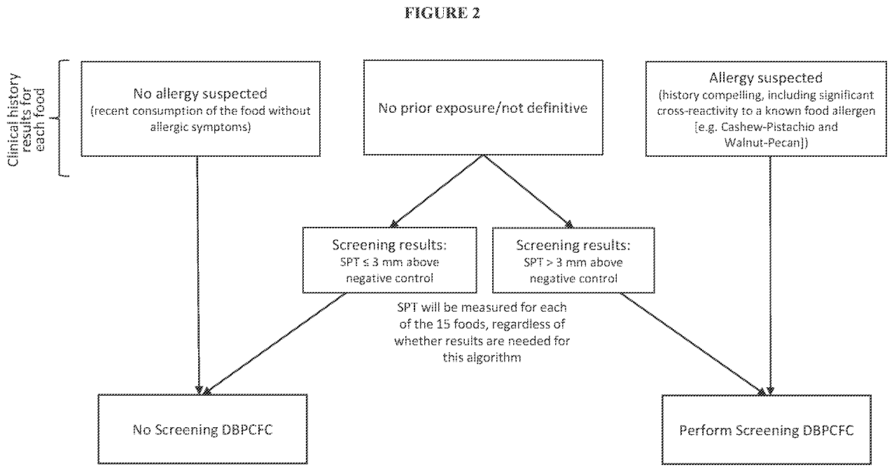

After providing informed consent with or without assent, as applicable, subjects will be screened to determine study eligibility over multiple visits (up to 12 weeks); initially, each subject will be screened for each of the 15 food sources of formulation A using the screening evaluation to determine DBPCFC testing as described in FIG. 2 and FIG. 3. Briefly, clinical FA history and skin-prick test (SPT) results will be obtained for each of the 15 food sources and used to identify foods to be evaluated during screening through DBPCFCs. Additional clinical history will be obtained from subjects on other allergens to understand the subject's broader allergic profile. During screening, subjects will undergo a Screening DBPCFC to a maximum challenge dose of 1000 mg for each potentially reactive food source. In order to qualify for study entry, each subject must have dose-limiting symptoms at or below the 100-mg level during the Screening DBPCFC to at least 1 and no more than 5 of the food sources contained in formulation A.

For each subject, each of the food sources contained within formulation A will be divided into 2 categories as shown in FIG. 3: (1) Reactive Food, defined as either a) Qualifying Food that elicits a reaction at ≤100 mg during the Screening DBPCFC, or b) Non-qualifying Food that elicits a reaction at >100 mg but ≤1000 mg during the Screening DBPCFC; or (2) Non-reactive Food, defined as foods that either a) do not meet clinical history and/or biomarker threshold criteria (SPT≤3 mm above negative control) to undergo a Screening DBPCFC, or b) meet criteria to undergo a Screening DBPCFC and are tolerated through the 1000-mg dose level at the Screening DBPCFC

Subjects who are found to meet the categorization of Reactive Foods that include qualifying food sources (to at least 1 and no more than 5 foods) will satisfy screening criteria to be randomized as long as they meet all other eligibility criteria.

At baseline (of the treatment period), eligible subjects will be randomized in a 2:2:1:1 ratio to 1 of 4 arms, with 2 arms in each dosing regimen (low or high), to receive daily oral doses of study drug (either formulation A or matching placebo) in a blinded fashion as shown in Table 2.

TABLE 2
Treatment Arms

| | | | | | Estimated Number of | |
|---|---|---|---|---|---|---|
| | | | | Estimated | Subjects per Age | |
| | | | | Number | Group Category | |
| Treatment | | | Target Dose | of | (years) | |
| Arm | Treatment | Regimen | Level | Subjects | ≥4 to <18 | ≥18 to ≤ 5 |
| 1 | formulation | Low-dose | 1500 mg/day | 24 | 20 | Up to 4 |
| | A | | (100 mg per food | | | |
| | | | source) | | | |
| 2 | formulation | High-dose | 4500 mg/day | 24 | 20 | Up to 4 |
| | A | | (300 mg per food | | | |
| | | | source) | | | |
| 3 | Placebo | Low-dose | (Content volume- | 12 | 10 | Up to 2 |
| | | | matched) | | | |
| 4 | Placebo | High-dose | (Content volume- | 12 | 10 | Up to 2 |
| | | | matched) | | | |

Baseline treatment will include oral administration of a single dose of study drug at 5 mg (equivalent to 0.3 mg/food source) under direct medical supervision at the study site, and, if tolerated, will continue at 5 mg/day at home for 2 weeks. Subjects who are unable to tolerate up to 3 attempts to administer study drug at dose levels below 50 mg during the updosing portion of the treatment period will discontinue study drug and continue with study assessments.

### Updosing Portion of the Treatment Period

Subjects will return to the clinic every 2 weeks for updosing under direct medical supervision until the randomized target dose of 1500 mg/day (100 mg per food source; low-dose regimen) or 4500 mg/day (300 mg per food source; high-dose regimen) is achieved (FIG. 1). Subjects who are unable to reach the randomized target dose after 3 attempts at updosing (at a given level) will continue on the highest dose level they are able to reach, as long as the dose of study drug is at least 50 mg. Subjects may updose through Week 38, at which point the Week 38 dose will be maintained through the end of the study. Therefore, the duration of updosing (during the treatment period) is anticipated to be of variable length depending on how quickly subjects are able to reach their randomized target dose or highest tolerated dose.

Failure to tolerate dose levels below 50 mg will lead to discontinuation of study drug

The general study drug updosing regimen is depicted in Table 3.

TABLE 3
Study Drug Updosing Regimen

| | | | | Minimum Approximate | Maximum Approximate |
|---|---|---|---|---|---|
| Total | | | | Total Amount of | Total Amount of |
| Protein | | | | Qualifying Reactive | Qualifying Reactive |
| FormA | | | | Food Source (for | Food Sources (for |
| per Day | Low-Dose | High-Dose | Increase | Subjects Allergic to | Subjects Allergic to |
| (mg) | Regimen | Regimen | (%) | 1 Food in FormA) (mg) | 5 foods in FormA) (mg) |
| 5 | X | X | — | 0.3 | 1.6 |
| 20 | X | X | 303 | 1.3 | 6.6 |
| 50 | X | X | 150 | 3.3 | 16.6 |
| 100 | X | X | 100 | 6.6 | 33.3 |
| 200 | X | X | 100 | 13.3 | 66.6 |
| 400 | X | X | 100 | 26.6 | 133.3 |
| 700 | X | X | 75 | 46.6 | 233.3 |
| 1000 | X | X | 43 | 66.6 | 333.3 |
| 1500 | X | X | 50 | 100 | 500 |
| 3000 | NA | X | 100 | 200 | 1000 |
| 4500 | NA | X | 50 | 300 | 1500 |

Abbreviations:
FormA = Formulation A;
NA = not applicable

The drug dose or updosing regimen may be modified based on adverse events and allergy symptom severity. Allergy symptom severity is depicted in Table

TABLE 4
CoFAR Severity Grading System for Allergic Reactions

| | | | Grade 4: Life- | Grade 5: |
|---|---|---|---|---|
| Grade 1: Mild | Grade 2: Moderate | Grade 3: Severe | Threatening | Death |
| Transient or mild | Symptoms that | Marked limitation | Extreme limitation | Death. |
| discomfort (<48 h), | produce mild to | in activity. Some | in activity. | |
| no or minimal | moderate | assistance is usually | Significant | |
| medical | limitation in | required; medical | assistance is | |
| intervention/therapy | activity. Some | intervention/therapy | required; | |
| required. | assistance might | required, and | significant | |
| These symptoms | be needed, but no | hospitalization is | medical/therapy | |
| can include | or minimal | possible. | is required. | |
| pruritus, swelling or | intervention/ | Symptoms can | Intervention is | |
| rash, abdominal | therapy is | include | required; | |
| discomfort, or other | required. | bronchospasm with | hospitalization is | |
| transient symptoms. | Hospitalization is | dyspnea, severe | probable. | |
| | possible. | abdominal pain, | Symptoms might | |
| | These symptoms | throat tightness | include persistent | |
| | can include | with hoarseness, | hypotension | |
| | persistent hives, | and transient | and/or hypoxia | |
| | wheezing without | hypotension, among | with resultant | |
| | dyspnea, | others. Parenteral | decreased level of | |
| | abdominal | medication(s) are | consciousness | |
| | discomfort/ | usually indicated. | associated with | |
| | increased | | collapse and/or | |
| | vomiting, or other | | incontinence or | |
| | symptoms. | | other life- | |
| | | | threatening | |
| | | | symptoms. | |

Abbreviations: CoFAR = Consortium of Food Allergy Research.
Source: Jones et al. (2017) Journal of Allergy and Clinical Immunology 139(4): 1242-
1252.e1249.

### Maintenance Portion of the Treatment Period

Once the appropriate target dose (or highest tolerated dose) is achieved, it will be maintained until Week 40. The maintenance portion of the treatment period may range from 2 to 22 weeks.

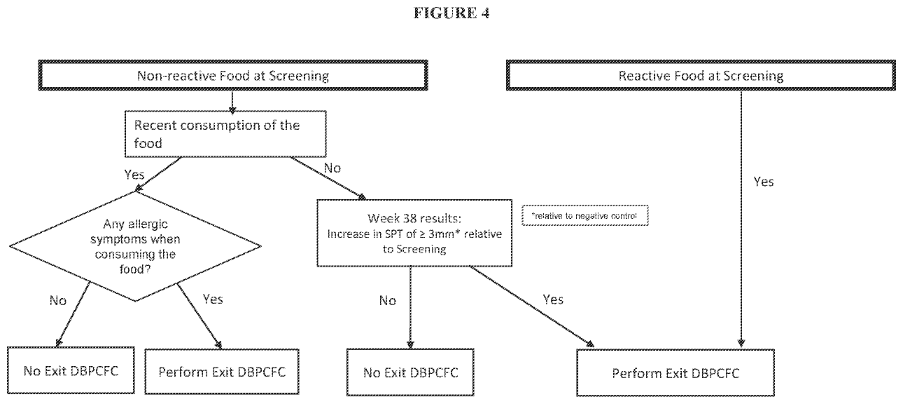

At the Week 40 visit, all subjects will undergo an Exit DBPCFC for all Reactive Foods determined during screening. The Week 40 visit will be done over multiple visits to allow for adequate separation of individual DBPCFC procedures. The Exit DBPCFCs (FIG. 4) will be performed in accordance with Practical Allergy (PRACTALL) guidelines (Sampson et al. (2012) J Allergy Clin Immunol 130(6): 1260-1274) and will require progression in an unaltered sequence, without repeating any dose. The Exit DBPCFCs will assess the same levels as done at study entry, as well as evaluating higher levels up to 4000 mg of protein of each reactive food source (see FIG. 4).

In order to obtain results before the Week 40 assessments begin, subjects will have an expanded study visit at Week 38. SPTs will be performed for all 15 foods. Foods that were determined to be Non-reactive Food at Screening will be reevaluated to assess for potential new FA development during the treatment period. Each Non-reactive Food at Screening will be assessed for any new clinical symptoms resulting from the food source; SPT results will also determine which additional foods, if any, will get an Exit DBPCFC, per FIG. 4. Only positive results on the DBPCFC will define a new FA developed during the treatment period.

Subjects will continue on study drug during the Exit DBPCFC period, but the daily study drug dose will be withheld on the day of the DBPCFC (±additional days per investigator judgment).

After the final Exit DBPCFC, all subjects will continue to receive study drug at their treatment dose for up to 4 weeks in a blinded treatment extension period, followed by the End of Treatment (EOT) visit.

Throughout the study, subjects will undergo safety and efficacy assessments.

The expected study duration is shown in Table 5.

TABLE 5
Expected Study Duration

| | |
|---|---|
| Screening period: | Up to 12 weeks |
| Treatment period: | Up to 50 weeks (includes 40 weeks of treatment |
| | plus up to 6 weeks for Exit DBPCFCs and 4 weeks |
| | of continued blinded study drug treatment through |
| | EOT); the treatment period will include an |
| | updosing portion and a maintenance portion, |
| | which will be variable per subject. |
| Follow-up period: | Up to 14 days from EOT |

### Inclusion Exclusion Criteria

Inclusion criteria include: (1) Aged 4 to 55 years (inclusive) at the time of signing the informed consent form (ICF); (2) Clinical history of allergy to at least 1 of the foods contained in formulation A (e.g., almond, cashew, chicken's egg, codfish, cow's milk, hazelnut, peanut, pecan, pistachio, salmon, sesame seed, shrimp, soy, walnut, and wheat); (3) Experience dose-limiting symptoms at or below the 100-mg dose level to at least 1 food source and no more than 5 food sources during the Screening DBPCFC; (4) All subjects who are of childbearing potential and their partners must agree to use highly effective contraception during the study. All subjects and their partners must continue to use highly effective contraception for 30 days after the last dose of study drug; (5) Female subjects of childbearing potential must have a negative serum pregnancy test at Screening and a negative urine pregnancy test at Baseline; (6) Female subjects not of childbearing potential must be either premenstrual or postmenopausal (defined as cessation of regular menstrual periods for at least 12 months) and are not required to use contraception; (7) Subject and/or legally authorized representative must be capable of giving signed informed consent, which includes compliance with the requirements and restrictions listed in the ICF; and (8) Minor subjects must be capable of giving written assent as appropriate per the applicable age (per local regulatory requirements).

Exclusion criteria include: (1) Confirmed presence of >5 FAs with dose-limiting symptoms at or below the 100-mg challenge dose level during the Screening DBPCFC of food sources contained in formulation A; (2) History of severe or life-threatening episode(s) of anaphylaxis or anaphylactic shock within 60 days of the first Screening DBPCFC; (3) History of EoE; other eosinophilic gastrointestinal disease; chronic, recurrent, or severe gastrointestinal reflux disease (GERD); symptoms of dysphagia (e.g., difficulty swallowing, food “getting stuck”); or recurrent gastrointestinal symptoms of undiagnosed etiology; (4) History of chronic disease (other than asthma, atopic dermatitis, or allergic rhinitis) that is, or is at significant risk of becoming, unstable or requiring a change in chronic therapeutic regimen; (5) Severe asthma per 2007 National Heart, Lung, and Blood Institute (NHLBI) Criteria Steps 5 or 6; (6) Mild or moderate asthma (2007 NHLBI Criteria Steps 1 through 4), if uncontrolled or difficult to control, as defined by any of the following; FEV1<80% of predicted, or ratio of FEV1 to forced vital capacity (FVC)<75% of predicted, with or without controller medications (only for age 6 years or greater and able to do spirometry), OR Inhaled corticosteroid (ICS) dosing of >500 μg daily fluticasone (or equivalent ICS based on NHLBI dosing chart, OR One hospitalization in the previous year for asthma prior to screening, OR Emergency room visit for asthma within 6 months prior to screening; (7) Known malignancy that is progressing or has required active treatment within the past 3 years (Subjects with basal cell carcinoma of the skin, squamous cell carcinoma of the skin, or carcinoma in situ (e.g., breast carcinoma, cervical cancer in situ) that have undergone potentially curative therapy are not excluded); (8) Known history of human immunodeficiency virus (HIV) (HIV 1/2 antibodies) (No testing for HIV is required unless mandated by the local health authority); (9) Known active hepatitis B infection (defined as hepatitis B surface antigen [HBsAg]-reactive) or known active hepatitis C virus (HCV) infection (defined as HCV RNA [qualitative] detected) (No testing for hepatitis B and hepatitis C is required unless mandated by the local health authority); (10) Uncontrolled diabetes (Subjects with controlled diabetes are allowed (insulin is allowed)); (11) Hypertension requiring >2 antihypertensive medications; (12) Known history of cardiovascular disease, including, but not limited to, history of myocardial infarction or arterial thromboembolic events within 6 months prior to enrollment, severe or unstable angina, New York Heart Association (NYHA) Class III or IV disease, or a history of corrected QT (QTc) interval >470 ms (Well-controlled hypertension is not considered to be part of this definition; see exclusion criterion 7 above); (13) History of interstitial lung disease; (14) History of confirmed mast cell disorder, including mastocytosis, urticaria pigmentosa, or hereditary or idiopathic angioedema; (15) History of celiac disease and/or any significant non-IgE-mediated intolerance (e.g., severe lactose intolerance) to 1 or more of the food sources contained in formulation A; (16) Active infection within 30 days of screening requiring systemic therapy; (17) Female subjects who are pregnant or breastfeeding, or expecting to conceive, or male subjects planning to father children within the projected duration of the study; (18) Active autoimmune disease that has required systemic treatment within 3 months (e.g., use of disease-modifying agents, corticosteroids, or immunosuppressive drugs); (19) Known psychiatric or substance-abuse disorders that would interfere with cooperation with the requirements of the study; (20) History or current evidence of any condition, therapy, or clinically significant laboratory abnormality that might preclude safe participation, confound the results of the study, or interfere with the subject's participation for the full duration of the study, or for which it is not in the best interest of the subject to participate, in the opinion of the treating investigator; (21) (History of regular steroid medication use (via intravenous [IV], intramuscular, or oral administration) in any of the following manners: History of daily oral steroid dosing for >1 month during the previous year or Burst oral steroid course in the previous 1 month or >2 burst oral steroid courses in the previous year ≥1 week in duration; (22) In the “build-up phase” of immunotherapy (i.e., has not reached maintenance dosing for at least 2 weeks) to another non-food allergen (e.g., environmental allergen, bee venom, etc.); (23) Inability to discontinue antihistamines at least 5 half-lives before SPT and DBPCFC; (24) Use of any therapeutic antibody (e.g., dupilumab, omalizumab, mepolizumab, reslizumab) currently or within the previous 6 months or 5 half-lives, whichever is longer; (25) Use of any food immunotherapy (e.g., oral, sublingual, epicutaneous) currently or within the previous 12 weeks; (26) Use of beta-blockers (oral), angiotensin-converting enzyme (ACE) inhibitors, angiotensin-receptor blockers (ARBs), or calcium-channel blockers; (27) Currently participating in or have participated in a study of an investigational agent within 4 weeks prior to Screening or 5 half-lives of the other investigational agent, whichever is longer; (28) Develops dose-limiting symptoms to placebo during the Screening DBPCFC; (29) Hypersensitivity to epinephrine or any of the excipients in study drug; and (30) Residing at the same address as another subject (e.g., siblings) participating in this or any other OIT/mOIT study.

### Assessments

### Double-Blind, Placebo-Controlled Food Challenge Procedure

The Double-Blind, Placebo-Controlled Food Challenge (DBPCFC) procedure used in this study, considered to be the gold standard for diagnosing FA, is based on the PRACTALL Consensus Guideline (Sampson et al. (2012) J Allergy Clin Immunol 130(6): 1260-1274); however, the updose schedules are slightly modified from the PRACTALL recommendations, and are presented in Table 6. The maximum dose will be 1000 mg for the Screening DBPCFC (FIG. 2) and 4000 mg for the Exit DBPCFC (FIG. 4).

Prior to initiating a DBPCFC, the following will occur:
Subjects will be asked to restrict the use of drugs that could interfere with the assessment of the DBPCFC, including, but not limited to, oral antihistamines (5 half-lives), beta-agonists (12 hours), theophylline (24 hours), and cromolyn (12 hours).
Subjects will have their oxygenation status recorded via pulse oximetry. Subject oxygen status must be >95% to continue with the food challenge procedure.
All subjects (aged 6 years or older) will undergo spirometry regardless of medical history and must have an FEV₁>80% of age predicted prior to starting the DBPCFC procedures. Subjects aged 4 to <6 years will undergo a pulmonary function assessment.
Subjects with a history of asthma will be assessed for worsening of asthma symptoms as determined by active wheezing or a PEFR<80% of predicted prior to each food challenge. If worsening asthma symptoms are present, the food challenge will be rescheduled until the subject is without wheezing and demonstrates pulmonary function >80% of age predicted level.
Subjects must be free from a flare of atopic disease (e.g., atopic dermatitis) or suspected intercurrent illness at the time of the DBPCFC.
Subjects must be fully recovered (back to their baseline state of health) from any preceding illness for at least 3 days, depending on investigator-determined suitability, to be able to continue in the study.

An independent physician who is not involved in the conduct of the study and other data collection beyond DBPCFC will oversee the DBPCFC. This physician will be blinded to the content of each food challenge, and will assess the subject's reactions at Screening and Exit DBPCFC. The order of the foods tested will be random and blinded for each subject in order to eliminate the potential for bias by the assessing personnel.

The food challenge is performed by feeding gradually increasing amounts of a suspected allergenic food, mixed with a palatable food, under physician observation in accordance with the PRACTALL consensus guidelines. If the study team suspects a reaction may be developing, it may exercise its clinical judgment to separate doses by up to an additional 30 minutes (1 hour maximum between doses). The placebo challenge will include placebo material given in the same number of doses and similar volume of dose material as in the active food sources (volumes of each of the active food challenges vary based upon the percentage protein content of the food).

If the subject begins to have any objective symptoms or subjective symptoms deemed significant per the PRACTALL guidelines, the food challenge will be terminated, and the subject will be given appropriate treatment.

If the subject has only mild symptoms related to the food source during the DBPCFC, that food source will be considered a Non-reactive Food for that subject.

TABLE 6
DBPCFC Doses at Screening and Exit

| Type of | | |
|---|---|---|
| Test | Screening | Exit |
| Doses | 1 | 1 |
| (mg) | 3 | 3 |
| | 10 | 10 |
| | 30 | 30 |
| | 100 | 100 |
| | 300 | 300 |
| | 600 | 600 |
| | 1000 | 1000 |
| | | 2000 |
| | | 4000 |
| Comments | The Screening DBPCFC will | The Exit DBPCFC will |
| | include 8 doses of food | include 10 doses of food |
| | source given every 15-30 | source given every 15-30 |
| | minutes in increasing | minutes in increasing |
| | amounts up to 1000 | amounts up to 4000 |
| | mg of food protein. | mg of food protein. |

Abbreviations: DBPCFC = double-blind, placebo-controlled food challenge.

At Screening, eligible subjects will be required to undergo a DBPCFC to specific food(s) if they meet any of the following criteria for an individual food source (see FIG. 2 and FIG. 3): (i) A positive clinical history, including significant cross-reactivity to a known FA, for example, to cashews if allergic to pistachios; or (ii) If the clinical history of a particular food is unknown because a subject has never consumed that food or has had no recent exposure to the food, then a DBPCFC will take place if a mean wheal diameter of >3 mm above negative control on an SPT at Screening is observed.

Subjects who complete updosing to at least the 50-mg dose level by Week 40, regardless of treatment assignment, will undergo Exit DBPCFC(s) as outlined below. All Reactive Foods (both Qualifying and Non-qualifying) will be challenged at the Exit DBPCFCs. Hence, an Exit DBPCFC will be triggered for the individual Non-reactive Foods at Screening when one of the following is present: (i) Allergic symptoms during recent food consumption; or (ii) For foods that were not recently consumed, an increase in SPT mean wheal diameter ≥3 mm (relative to negative control) at Week 38 relative to screening.

### Patient-Reported Outcomes

The impact of FAs manifests in fear, anxiety, and other effects that can only be measured by directly asking the subject. In order to understand how formulation A affects a subject's FA-related QoL over time, the patient reported outcomes (PROs) listed below will be administrated.

The Food Allergy Quality of Life Questionnaire (FAQLQ) is a disease-specific questionnaire for subjects with FAs. FAQLQ versions have demonstrated good reliability and validity and have shown responsiveness when changes occur (Flokstra-de Blok et al. (2009) Allergy 64(8): 1209-1217; van der Velde et al. (2009) Qual Life Res 18(2): 245-251; DunnGalvin et al. (2010) Clin Exp Allergy 40(3): 476-485; Wassenberg et al. (2012) Pediatr Allergy Immunol 23(5): 412-419)

The Accidental Exposure Questionnaire is a self-administered questionnaire containing 11 items asking about known food allergen exposure, associated symptoms experienced (if any), and treatment received (if any) following accidental exposure to a food to which the subject is allergic.

The Immunotherapy-Related Quality of Life (IQoL) is a questionnaire designed to assess aspects of health-related quality of life (HRQoL) affected by living with an FA. In contrast to the FAQLQ, the IQoL aims to assess perceived impairment resulting from allergy to foods specifically treated by OIT. The IQoL assesses constructs identified by OIT-treated patients as contributing to psychosocial burden in the context of their food allergen immunotherapy.

The Treatment Satisfaction Questionnaire for Medication (TSQM-9) is a well-validated, self-administered, 9-item questionnaire developed to assess the satisfaction that patients had with their medical treatment (Bharmal et al. (2009) Health Qual Life Outcomes 7: 36).

The Food Allergy Quality of Life-Parental Burden Questionnaire (FAQL-PB) is a self-administered 17-item questionnaire developed to measure the burden on parents with food-allergic children aged 0 to 17 years (Cohen et al. (2004) J Allergy Clin Immunol 114(5): 1159-1163).

### Biomarkers

Biomarkers that may improve understanding of the biological mechanisms of FA and the impact of treatment with OIT, as well as potentially help identify subjects who may benefit from OIT, will be measured in this study. Hence, biomarker results from this study may benefit future efforts to identify noninvasive markers for FA diagnosis, predictors of DBPCFC outcomes, and response to OIT with formulation A.

The following blood biospecimens will be collected. Some markers will be analyzed throughout the course of the study, and others will be stored for batched biomarker analysis later in the study:
Serum for sIgE levels for all 15 foods contained in formulation A.
Serum for measurements of other relevant biomarkers, including, but not limited to, total IgE, total and specific IgG4, cytokines, and microRNA.
Whole blood for isolation of peripheral blood mononuclear cells (PBMCs), which will be stored and analyzed for various immune-related biomarkers, including, but not limited to, T cell activation and repertoire.
Whole blood to analyze biomarkers related to, but not limited to, epigenetic changes in the DNA and T cell repertoire determination.

Stool samples will be used to determine the microbiome dysbiosis in subjects with FA and impact of OIT on the microbiome pre- and post-OIT with formulation A.

Blood samples collected and stored in this study may also be used to characterize the sensitization profile for different allergenic proteins, including, but not limited to, foods not included in formulation A, pollen, grass, insect venom, mites, cockroaches, dander, and epithelia.

### Safety Assessments

Safety assessments will include physical examinations. A complete physical examination will include, at a minimum, assessments of the skin and cardiovascular, respiratory, gastrointestinal, and neurological systems. Height and weight will also be measured and recorded. A brief symptom-directed physical examination will include, at a minimum, assessments of the skin and cardiovascular, respiratory, gastrointestinal, and neurological systems. The subject will also be assessed for specific signs and symptoms of EoE. Vital signs are to be taken prior to any blood draw that occurs at the same time point/visit.

### Additional Assessments

Subjects will have Skin Prick Tests (SPTs) performed for food allergens. Prior to the SPT, the investigator must ensure that the subject has not received antihistamine medications for at least 5 half-lives of the medication. Commercial allergenic extract solution is introduced to the epidermis via lancet or skin prick for each of the 15 food sources to determine sensitivity to select foods that may be responsible for triggering symptoms in subjects with FA. In addition to allergens, positive (histamine) and negative (saline-glycerin) controls are also introduced to establish that the response is not blocked and to determine whether there is dermatographism, respectively. The positive and negative control tests should be performed and measured along with the allergen SPT.

Asthma severity will be assessed during screening and at the Week 38 visit using the NHLBI Guidelines for the Diagnosis and Management of Asthma (Expert Panel Report 3 [EPR-3]) (National Asthma Education Prevention Program (2007) J Allergy Clin Immunol 120(5 Suppl): S94-138; Urbano et al. (2008) J Manag Care Pharm 14(1): 41-49). Assessment of asthma control in asthmatic subjects using the Asthma Control Test (ACT) questionnaire (Schatz et al. (2006) J Allergy Clin Immunol 117(3): 549-556) or Childhood Asthma Control Test (C-ACT) questionnaire (Bime et al. (2016) NPJ Prim Care Respir Med 26: 16075) will be performed.

### Example 2

The protocol described in Example 1 may be adapted to allow for at-home updosing for subjects with mild to moderate food allergies. At-home updosing in certain circumstances could improve feasibility of frequent visits and/or give greater access to relevant medical support, thereby improving patient convenience and overall access to care.

All aspects of the protocol described in Example 1, including the study drug, objectives and endpoints, overall design, inclusion/exclusion criteria, and assessments would apply, except where indicated otherwise in this Example or where inconsistent with the goal of at-home updosing for subjects with mild to moderate food allergies.

Subjects have mild to moderate food allergy, which may be characterized by, for example, one or more of the following: a higher eliciting dose for a food during a screening DBPCFC relative to a subject with moderate to severe food allergy (e.g., an eliciting dose ≥100 mg); a modest co-morbid disease (e.g., no or well-controlled asthma); and well-controlled food allergy symptoms but impaired quality of life. Subjects may be mono- or multi-allergic, adults or pediatric. Exclusion criteria include no severe asthma (if the subject is asthmatic, it is well controlled) and no prior ER or ICU visits due to food allergy.

A first treatment dose is administered in-clinic. The following visits, approximately every two weeks, are conducted by telemedicine. Telemedicine is also used for up-dosing. Telemedicine visits are accompanied by support tools, reaction management (e.g., epinephrine), and detailed training and educational resources. Discretionary in-clinic visits, including for updosing, are permitted based on joint decision making of the clinician and subject or family in the context of the overall clinical picture.

Primary outcomes include descriptive statistics for adverse event (AE) outcomes, quality of life (QOL), and adherence. Exploratory outcomes include comparison to historical AE, QOL, and adherence. Subjects may not require an exit food challenge. Subjects may roll over to a common open-label extension (OLE).

### INCORPORATION BY REFERENCE

All publications and patents mentioned herein, including those items listed below, are hereby incorporated by reference in their entirety for all purposes as if each individual publication or patent was specifically and individually incorporated by reference. In case of conflict, the present application, including any definitions herein, will control.

### EQUIVALENTS

While specific embodiments of the subject disclosure have been discussed, the above specification is illustrative and not restrictive. Many variations of the disclosure will become apparent to those skilled in the art upon review of this specification. The full scope of the disclosure should be determined by reference to the claims, along with their full scope of equivalents, and the specification, along with such variations.

What is claimed is:

1. A method for treating or preventing an allergy in a subject in need thereof, the method comprising orally administering to the subject a daily dose of a mixed allergen composition for at least about 40 weeks, wherein the daily dose is increased about every two to six weeks from an initial daily dose of about 5 mg (by total protein weight) of the mixed allergen composition to a final daily dose that is the lower of about 4500 mg (by total protein weight) of the mixed allergen composition or a highest tolerated dose of the mixed allergen composition.

2. A method of increasing the tolerance threshold of a food product in a subject having an allergy to the food product, the method comprising administering to the subject a daily dose of a mixed allergen composition for at least about 40 weeks, wherein the daily dose is increased about every two to six weeks from an initial daily dose of about 5 mg (by total protein weight) of the mixed allergen composition to a final daily dose that is the lower of about 4500 mg (by total protein weight) of the mixed allergen composition or a highest tolerated dose of the mixed allergen composition.

3. The method of claim 1 or 2, wherein the subject has a food allergy to only one food product.

4. The method of claim 1 or 2, wherein the subject has a food allergy to from two to fifteen different food products.

5. The method of claim 4, wherein the subject has a food allergy to from two to ten or two to five different food products.

6. The method of any one of claims 1-5, wherein the daily dose of the mixed allergen composition is increased about every two to six weeks from the initial daily dose of about 5 mg to about 20 mg to about 50 mg to about 100 mg to about 200 mg to about 400 mg to about 700 mg to about 1000 mg to about 1500 mg to about 3000 mg to the final daily dose of about 4500 mg.

7. A method for treating or preventing an allergy in a subject in need thereof, the method comprising orally administering to the subject a daily dose of a mixed allergen composition for at least about 40 weeks, wherein:
(i) for about the first two to six weeks, the daily dose of the mixed allergen compositions is about 5 mg (by total protein weight);
(ii) for about the next two to six weeks, the daily dose of the mixed allergen compositions is about 20 mg;
(iii) for about the next two to six weeks, the daily dose of the mixed allergen compositions is about 50 mg;
(iv) for about the next two to six weeks, the daily dose of the mixed allergen compositions is about 100 mg;
(v) for about the next two to six weeks, the daily dose of the mixed allergen compositions is about 200 mg;
(vi) for about the next two to six weeks, the daily dose of the mixed allergen compositions is about 400 mg;
(vii) for about the next two to six weeks, the daily dose of the mixed allergen compositions is about 700 mg;
(viii) for about the next two to six weeks, the daily dose of the mixed allergen compositions is about 1000 mg;
(ix) for about the next two to six weeks, the daily dose of the mixed allergen compositions is about 1500 mg;
(x) for about the next two to six weeks, the daily dose of the mixed allergen compositions is about 3000 mg; and
(xi) for about the next twenty weeks, the daily dose of the mixed allergen compositions is about 4500 mg;
wherein if the subject cannot tolerate a higher dose the dose is not increased and the subject is administered a highest tolerated dose for the remainder of the at least about 40 weeks.

8. A method for treating or preventing an allergy in a subject in need thereof, the method comprising orally administering to the subject a daily dose of a mixed allergen composition for at least about 40 weeks, wherein:
(i) for about the first two to six weeks, the daily dose of the mixed allergen compositions is about 5 mg (by total protein weight);
(ii) for about the next two to six weeks, the daily dose of the mixed allergen compositions is about 20 mg;
(iii) for about the next two to six weeks, the daily dose of the mixed allergen compositions is about 50 mg;
(iv) if the subject can tolerate a daily dose of about 100 mg, for about the next two to six weeks, the daily dose of the mixed allergen compositions is about 100 mg, or, if the subject cannot tolerate a daily dose of about 100 mg, for about the next 30 to 34 weeks, the daily dose of the mixed allergen composition is about 50 mg;
(v) if the subject can tolerate a daily dose of about 200 mg, for about the next two to six weeks, the daily dose of the mixed allergen compositions is about 200 mg, or, if the subject cannot tolerate a daily dose of about 200 mg, for about the next 28 to 32 weeks, the daily dose of the mixed allergen composition is about 100 mg;
(vi) if the subject can tolerate a daily dose of about 400 mg, for about the next two to six weeks, the daily dose of the mixed allergen compositions is about 400 mg, or, if the subject cannot tolerate a daily dose of about 400 mg, for about the next 26 to 30 weeks, the daily dose of the mixed allergen composition is about 200 mg;
(vii) if the subject can tolerate a daily dose of about 700 mg, for about the next two to six weeks, the daily dose of the mixed allergen compositions is about 700 mg, or, if the subject cannot tolerate a daily dose of about 700 mg, for about the next 24 to 28 weeks, the daily dose of the mixed allergen composition is about 400 mg;
(viii) if the subject can tolerate a daily dose of about 1000 mg, for about the next two to six weeks, the daily dose of the mixed allergen compositions is about 1000 mg, or, if the subject cannot tolerate a daily dose of about 1000 mg, for about the next 22 to 26 weeks, the daily dose of the mixed allergen composition is about 700 mg;
(ix) if the subject can tolerate a daily dose of about 1500 mg, for about the next two to six weeks, the daily dose of the mixed allergen compositions is about 1500 mg, or, if the subject cannot tolerate a daily dose of about 1500 mg, for about the next 20 to 24 weeks, the daily dose of the mixed allergen composition is about 1000 mg;
(x) if the subject can tolerate a daily dose of about 3000 mg, for about the next two to six weeks, the daily dose of the mixed allergen compositions is about 3000 mg, or, if the subject cannot tolerate a daily dose of about 3000 mg, for about the next 18 to 22 weeks, the daily dose of the mixed allergen composition is about 1500 mg;
(xi) if the subject can tolerate a daily dose of about 4500 mg, for about the next 16 to 20 weeks, the daily dose of the mixed allergen compositions is about 4500 mg, or, if the subject cannot tolerate a daily dose of about 4500 mg, for about the next 16 to 20 weeks, the daily dose of the mixed allergen composition is about 3000 mg.

9. A method for treating or preventing an allergy in a subject in need thereof, the method comprising:
(i) orally administering to the subject a daily dose of about 5 mg (by total protein weight) of a mixed allergen composition for at least about two to six weeks;
(ii) orally administering to the subject a daily dose of about 20 mg of a mixed allergen composition for at least about two to six weeks;
(iii) orally administering to the subject a daily dose of about 50 mg of a mixed allergen composition for at least about two to six weeks;
(iv) orally administering to the subject a daily dose of about 100 mg of a mixed allergen composition for at least about two to six weeks;
(v) orally administering to the subject a daily dose of about 200 mg of a mixed allergen composition for at least about two to six weeks;
(vi) orally administering to the subject a daily dose of about 400 mg of a mixed allergen composition for at least about two to six weeks;
(vii) orally administering to the subject a daily dose of about 700 mg of a mixed allergen composition for at least about two to six weeks;
(viii) orally administering to the subject a daily dose of about 1000 mg of a mixed allergen composition for at least about two to six weeks;
(ix) orally administering to the subject a daily dose of about 1500 mg of a mixed allergen composition for at least about two to six weeks;
(x) orally administering to the subject a daily dose of about 3000 mg of a mixed allergen composition for at least about two to six weeks; and/or
(xi) orally administering to the subject a daily dose of about 4500 mg of a mixed allergen composition for at least about two to six weeks.

10. The method of any one of claims 1-9, wherein the method further comprises orally administering to the subject a daily dose of the mixed allergen composition that is the lower of about 4500 mg of the mixed allergen composition or a highest tolerated dose of the mixed allergen composition for at least about the next 1, 2, 3, or 4 weeks, 1, 2, 3, 4, 5, 6, 9, or 12 months or 1, 2, 3, 4, 5 years.

11. A method for treating or preventing an allergy in a subject in need thereof, the method comprising orally administering to the subject a daily dose of a mixed allergen composition for at least about 40 weeks, wherein the daily dose is increased about every two to six weeks from an initial daily dose of about 0.3 mg protein from each food source in the mixed allergen composition to a final daily dose that is the lower of about 300 mg of protein from each food source in the mixed allergen composition or a highest tolerated dose of the mixed allergen composition.

12. A method of increasing the tolerance threshold of a food product in a subject having an allergy to the food product, the method comprising orally administering to the subject a daily dose of a mixed allergen composition for at least about 40 weeks, wherein the daily dose is increased about every two to six weeks from an initial daily dose of about 0.3 mg protein from each food source in the mixed allergen composition to a final daily dose that is the lower of about 300 mg of protein from each food source in the mixed allergen composition or a highest tolerated dose of the mixed allergen composition.

13. The method of claim 11 or 12, wherein the daily dose of the mixed allergen composition is increased about every two to six weeks from the initial daily dose of about 0.3 mg protein from each food source in the mixed allergen composition to about 1.3 mg to about 3.3 mg to about 6.6 mg to about 13.3 mg to about 26.6 mg to about 846.6 mg to about 66.6 mg to about 100 mg to about 200 mg to the final daily dose of about 300 mg protein from each food source in the mixed allergen composition.

14. A method for treating or preventing an allergy in a subject in need thereof, the method comprising orally administering to the subject a daily dose of a mixed allergen composition for at least about 40 weeks, wherein
(i) for about the first two to six weeks, the daily dose of the mixed allergen compositions is about 0.3 mg protein from each food source in the mixed allergen composition;
(ii) for about the next two to six weeks, the daily dose of the mixed allergen compositions is about 1.3 mg protein from each food source in the mixed allergen composition;
(iii) for about the next two to six weeks, the daily dose of the mixed allergen compositions is about 3.3 mg protein from each food source in the mixed allergen composition;
(iv) for about the next two to six weeks, the daily dose of the mixed allergen compositions is about 6.6 mg protein from each food source in the mixed allergen composition;
(v) for about the next two to six weeks, the daily dose of the mixed allergen compositions is about 13.3 mg protein from each food source in the mixed allergen composition;
(vi) for about the next two to six weeks, the daily dose of the mixed allergen compositions is about 26.6 mg protein from each food source in the mixed allergen composition;
(vii) for about the next two to six weeks, the daily dose of the mixed allergen compositions is about 46.6 mg protein from each food source in the mixed allergen composition;
(viii) for about the next two to six weeks, the daily dose of the mixed allergen compositions is about 66.6 mg protein from each food source in the mixed allergen composition;
(ix) for about the next two to six weeks, the daily dose of the mixed allergen compositions is about 100 mg protein from each food source in the mixed allergen composition;
(x) for about the next two to six weeks, the daily dose of the mixed allergen compositions is about 200 mg protein from each food source in the mixed allergen composition; and
(xi) for about the next twenty weeks, the daily dose of the mixed allergen compositions is about 300 mg protein from each food source in the mixed allergen composition;
wherein if the subject cannot tolerate a higher dose the dose is not increased and the subject is administered a highest tolerated dose for the remainder of the at least about 40 weeks.

15. A method for treating or preventing an allergy in a subject in need thereof, the method comprising orally administering to the subject a daily dose of a mixed allergen composition for at least about 40 weeks, wherein the daily dose is increased about every two to six weeks from an initial daily dose of about 5 mg (by total protein weight) of the mixed allergen composition to a final daily dose that is the lower of about 1500 mg of the mixed allergen composition or a highest tolerated dose of the mixed allergen composition.

16. A method of increasing the tolerance threshold of one or more food products and/or environmental allergens in a subject having an allergy to the food product or environmental allergen, the method comprising orally administering to the subject a daily dose of a mixed allergen composition for at least about 40 weeks, wherein the daily dose is increased about every two to six weeks from an initial daily dose of about 5 mg (by total protein weight) of the mixed allergen composition to a final daily dose that is the lower of about 1500 mg of the mixed allergen composition or a highest tolerated dose of the mixed allergen composition.

17. The method of claim 15 or 16, wherein the daily dose of the mixed allergen composition is increased about every two to six weeks from the initial daily dose of about 5 mg to about 20 mg to about 50 mg to about 100 mg to about 200 mg to about 400 mg to about 700 mg to about 1000 mg to the final daily dose of about 1500 mg.

18. A method for treating or preventing an allergy in a subject in need thereof, the method comprising orally administering to the subject a daily dose of a mixed allergen composition for at least about 40 weeks, wherein
(i) for about the first two to six weeks, the daily dose of the mixed allergen compositions is about 5 mg (by total protein weight);
(ii) for about the next two to six weeks, the daily dose of the mixed allergen compositions is about 20 mg;
(iii) for about the next two to six weeks, the daily dose of the mixed allergen compositions is about 50 mg;
(iv) for about the next two to six weeks, the daily dose of the mixed allergen compositions is about 100 mg;
(v) for about the next two to six weeks, the daily dose of the mixed allergen compositions is about 200 mg;
(vi) for about the next two to six weeks, the daily dose of the mixed allergen compositions is about 400 mg;
(vii) for about the next two to six weeks, the daily dose of the mixed allergen compositions is about 700 mg;
(viii) for about the next two to six weeks, the daily dose of the mixed allergen compositions is about 1000 mg; and
(ix) for about the next 24 weeks, the daily dose of the mixed allergen compositions is about 1500 mg;
wherein if the subject cannot tolerate a higher dose the dose is not increased and the subject is administered a highest tolerated dose for the remainder of the at least about 40 weeks.

19. The method of any one of claims 1-18, wherein the mixed allergen composition comprises an allergen from at least 6 different food sources;

20. The method of claim 19, wherein the mixed allergen composition comprises an allergen from at least 7, 8, 9, 10, 11, 12, 13, 14, or 15 different food sources;

21. The method of claim 20, wherein the mixed allergen composition comprises an allergen from 15 different food sources.

22. The method of any one of claims 1-21, wherein the mixed allergen composition comprises an almond, cashew, cod, egg, hazelnut, milk, peanut, pecan, pistachio, salmon, sesame, shrimp, soy, walnut, or wheat (e.g., wheat gluten) allergen, or any combination thereof.

23. The method of claim 22, wherein the mixed allergen composition comprises an almond, cashew, cod, egg, hazelnut, milk, peanut, pecan, pistachio, salmon, sesame, shrimp, soy, walnut, and wheat (e.g., wheat gluten) allergen.

24. The method of claim 23, wherein the mixed allergen composition comprises almond protein, cashew protein, cod powder, dried eggs, hazelnut flour, milk protein, peanut flour, pecan flour, pistachio flour, salmon powder, sesame flour, shrimp powder, soy protein, walnut flour, and wheat (e.g., wheat gluten).

25. The method of any one of claims 1-24, wherein the mixed composition comprises about equal parts per weight of protein from each food source present in the mixed allergen composition.

26. The method of any one of claims 1-25, wherein one or more of the daily doses comprises:
about 0.3 mg, about 1.3 mg, about 6.6 mg, about 13.3 mg, about 26.6 mg, about 46.6 mg, about 66.6 mg about 100 mg, about 200 mg, or about 300 mg of protein from each food source present in the mixed allergen composition; and/or
about 5 mg, about 20 mg, about 50 mg, about 100 mg, about 200 mg, about 400 mg, about 700 mg, about 1000 mg, about 1500 mg, about 3000 mg, or about 4000 mg of total food protein.

27. The method of any of claims 1-26, wherein the method increases the amount of an allergic food that the subject can tolerate, as measured by a double-blind, placebo-controlled food challenge (DBPCFC).

28. The method of claim 27, wherein the method increases the highest amount of an allergenic food that the subject can tolerate in a DBPCFC from less than or equal to about 100 mg (by protein weight) to greater than or equal to about 600 mg.

29. The method of claim 27, wherein the method increases the highest amount of an allergenic food that the subject can tolerate in a DBPCFC from less than or equal to about 100 mg (by protein weight) to greater than or equal to about 1000 mg.

30. The method of claim 27, wherein the method increases the total amount of an allergenic food that the subject can tolerate in a DBPCFC from less than or equal to about 100 mg (by protein weight) to greater than or equal to about 1000 mg.

31. The method of claim 27, wherein the method increases the total amount of an allergenic food that the subject can tolerate in a DBPCFC from less than or equal to about 100 mg (by protein weight) to greater than or equal to about 2000 mg.

32. The method of any one of claims 1-31, wherein the method increases the amount of two or more allergic foods that the subject can tolerate.

33. The method of any one of claims 1-32, wherein the subject is from about 4 years to about 55 years old.

34. The method of any one of claims 1-33, wherein the subject is a pediatric subject.

35. The method of any one of claims 1-34, wherein the subject is allergenic to one or more foods selected from almond, cashew, chicken's egg, codfish, cow's milk, hazelnut, peanut, pecan, pistachio, salmon, sesame seed, shrimp, soy, walnut, and wheat (e.g., wheat gluten).

36. The method of any one of claims 1-35, wherein the subject cannot tolerate 100 mg (by protein weight), as measured by DBPCFC, of one or more foods selected from almond, cashew, chicken's egg, codfish, cow's milk, hazelnut, peanut, pecan, pistachio, salmon, sesame seed, shrimp, soy, walnut, and wheat (e.g., wheat gluten).

37. The method of any one of claims 1-36, wherein the subject is allergic to at most five foods selected from almond, cashew, chicken's egg, codfish, cow's milk, hazelnut, peanut, pecan, pistachio, salmon, sesame seed, shrimp, soy, walnut, and wheat (e.g., wheat gluten).

38. The method of any one of claims 1-37, wherein the subject cannot tolerate 100 mg (by protein weight), as measured by DBPCFC, of at most five foods selected from almond, cashew, chicken's egg, codfish, cow's milk, hazelnut, peanut, pecan, pistachio, salmon, sesame seed, shrimp, soy, walnut, and wheat (e.g., wheat gluten).

39. The method of any one of claims 1-38, wherein the subject has not had a severe/life-threatening episode of anaphylaxis/anaphylactic shock within 60 days of receiving the initial dose of the mixed allergen composition.

40. The method of any one of claims 1-39, wherein the subject does not have a history of eosinophilic esophagitis (EoE), other eosinophilic gastrointestinal disease, chronic, recurrent, or severe gastrointestinal reflux disease (GERD), symptoms of dysphagia (e.g., difficulty swallowing, food “getting stuck”), or recurrent gastrointestinal symptoms of undiagnosed etiology

41. The method of any one of claims 1-40, wherein the subject does not have a history of chronic disease (other than asthma, atopic dermatitis, or allergic rhinitis) that is, or is at significant risk of becoming unstable or requiring a change in chronic therapeutic regimen.

42. The method of any one of claims 1-41, wherein the subject has not had an active infection within 30 days of receiving the initial dose of the mixed allergen composition.

43. The method of any one of claims 1-42, wherein (i) one or more of the doses of the mixed allergenic composition are not administered at a clinic, (ii) one or more of the doses of the mixed allergen composition are administered at the subject's home, and/or (iii) one or more of the doses of the mixed allergen composition are administered via telemedicine.

44. The method of any one of claims 1-43, wherein (i) a dose of the mixed allergen composition is increased not at a clinic, (ii) a dose of the mixed allergen composition is increased at the subject's home, and/or (iii) a dose of the mixed allergen composition is increased via telemedicine.

45. The method of claim 43 or 44, wherein the subject has a mild to moderate food allergy.

46. A kit comprising:
(i) at least about 14 five mg (by total protein weight) doses of a mixed allergen composition;
(ii) at least about 14 20 mg doses of the mixed allergen composition;
(iii) at least about 14 50 mg doses of the mixed allergen composition;
(iv) at least about 14 100 mg doses of the mixed allergen composition;
(v) at least about 14 200 mg doses of the mixed allergen composition;
(vi) at least about 14 400 mg doses of the mixed allergen composition;
(vii) at least about 14 700 mg doses of the mixed allergen composition;
(viii) at least about 14 1000 mg doses of the mixed allergen composition;
(ix) at least about 14 1500 mg doses of the mixed allergen composition;
(x) at least about 14 3000 mg doses of the mixed allergen composition; and/or
(xi) at least about 14 4500 mg doses of the mixed allergen composition.

47. The kit of claim 46, wherein the kit comprises:
(i) at least about 21 five mg doses of a mixed allergen composition;
(ii) at least about 21 20 mg doses of the mixed allergen composition;
(iii) at least about 21 50 mg doses of the mixed allergen composition;
(iv) at least about 21 100 mg doses of the mixed allergen composition;
(v) at least about 21 200 mg doses of the mixed allergen composition;
(vi) at least about 21 400 mg doses of the mixed allergen composition;
(vii) at least about 21 700 mg doses of the mixed allergen composition;
(viii) at least about 21 1000 mg doses of the mixed allergen composition;
(ix) at least about 21 1500 mg doses of the mixed allergen composition;
(x) at least about 21 3000 mg doses of the mixed allergen composition; and/or
(xi) at least about 21 4500 mg doses of the mixed allergen composition.

48. A kit comprising:
(i) at least about 14 five mg (by total protein weight) doses of a mixed allergen composition;
(ii) at least about 14 20 mg doses of the mixed allergen composition;
(iii) at least about 14 50 mg doses of the mixed allergen composition;
(iv) at least about 14 100 mg doses of the mixed allergen composition;
(v) at least about 14 200 mg doses of the mixed allergen composition;
(vi) at least about 14 400 mg doses of the mixed allergen composition;
(vii) at least about 14 700 mg doses of the mixed allergen composition;
(viii) at least about 14 1000 mg doses of the mixed allergen composition; and/or
(ix) at least about 14 1500 mg doses of the mixed allergen composition.

49. The kit of claim 48, wherein the kit comprises:
(i) at least about 21 five mg doses of a mixed allergen composition;
(ii) at least about 21 20 mg doses of the mixed allergen composition;
(iii) at least about 21 50 mg doses of the mixed allergen composition;
(iv) at least about 21 100 mg doses of the mixed allergen composition;
(v) at least about 21 200 mg doses of the mixed allergen composition;
(vi) at least about 21 400 mg doses of the mixed allergen composition;
(vii) at least about 21 700 mg doses of the mixed allergen composition;
(viii) at least about 21 1000 mg doses of the mixed allergen composition; and/or
(ix) at least about 21 1500 mg doses of the mixed allergen composition.
